(19) United States
(12) Patent Application Publication
(10) Pub. No.: US 2024/0384319 A1
(43) Pub. Date: Nov. 21, 2024

(54) Synthesis of Oligonucleotides

(71) Applicant: The University of Manchester, Manchester (GB)

(72) Inventor: Sarah Lovelock, Manchester (GB)

(21) Appl. No.: 18/692,739

(22) PCT Filed: Sep. 16, 2022
(86) PCT No.: PCT/GB2022/052358

§ 371 Date: Mar. 15, 2024

(30) Foreign Application Priority Data
Sep. 17, 2021 (GB) 2113340.0

(51) Int. Cl. C12P 19/34; C12N 9/12; C12N 15/11

(52) U.S. Cl. CPC C12P 19/34; C12N 9/1247; C12N 9/1252; C12N 15/111; C12N 2330/30; C12Y 207/07006; C12Y 207/07007

(57) ABSTRACT

The present invention relates to a novel method for oligonucleotide synthesis, using a catalytic primer-template, e.g. a hairpin primer, to achieve multiple rounds of oligonucleotide synthesis and dissociation from the template in the same reaction, such that at the end of the reaction the amount of oligonucleotide generated is greater than the amount of template provided in the reaction. The primer contains a cleavable site and the reaction is carried out in a single mixture with a cleavage agent. The cleavable site may be an inosine and the agent an endonuclease V.

The present invention relates to a method for synthesising an oligonucleotide, in particular a single stranded oligonucleotide. The present invention also relates to an oligonucleotide produced by a method of the invention. The present invention relates to a kit of parts for synthesising an oligonucleotide, and a support for synthesising an oligonucleotide. The present invention also relates to a reaction comprising a primer/template, a polymerase and a cleaving agent. The present invention also relates to a cell comprising a nucleic acid sequence or vector encoding a primer/template of the invention.

### BACKGROUND

Therapeutic oligonucleotides which bind to mRNA to modulate the production of disease related proteins have emerged as a new drug modality for the treatment of a whole range of disease areas (Khvorova et al. Nat. Biotechnol. 2017, 35, 238). Recent years have seen significant investment in therapeutic oligonucleotides, following the award of the Nobel Prize for the discovery of RNA interfering technology and FDA approval of several RNA-based therapeutics for the treatment of rare diseases. There are currently more than 160 oligonucleotide products in clinical trials including those for population based indications (Hugget et al. Nat. Biotechnol. 2017, 35, 708). The increase in the number of potential therapeutics, including those for common diseases, creates a significant manufacturing challenge as existing methods of chemical synthesis are restricted to 10 Kg batches and are not suitable for large scale applications (>100 Kg) (Tedebark et al. Methods Mol. Biol. 2011, 683, 505).

Therapeutic oligonucleotides are short DNA analogues which selectively bind to target mRNA through Watson-Crick base pairing to regulate the production of disease related proteins. They include double stranded RNA molecules known as small interfering RNAs (siRNAs) and single stranded molecules known as antisense oligonucleotides (ASOs). Whilst oligonucleotides have been studied as potential drug molecules for more than 40 years progress in translating them into successful therapeutics has been limited by insufficient biological efficacy, bioavailability and off-pathway toxic effects. In recent years a number of chemical modifications have been reported which substantially improve oligonucleotide delivery, metabolic stability and potency including substitutions to the DNA backbone, pyrimidine bases and ribose sugars (FIG. 1) (Khvorova et al supra; Rinaldi et al. Nat. Rev. Neurol. 2018, 1, 9;., and Smith et al. Annu. Rev. Pharmacol. Toxicol. 2019, 59, 605; Dowdy, Nat. Biotechnol. 2017, 35, 222).

Therapeutic oligonucleotides may require chemical modifications to confer improved efficacy, selectivity, metabolic stability, and toxicity profiles (Rinaldi et al. Nat. Rev. Neurol. 2018, 1, 9; Smith et al. Annu. Rev. Pharmacol. Toxicol. 2019, 59, 605). While natural polymerases are tolerant to some nucleotide modifications (Nakamaye et al Nucleic Acids Res. 1988, 21, 9947; Yang et al. Nucleic Acids Res. 2007, 35, 3118; Romaniuk et al. J. Biol. Chem. 1982, 257, 7684) their activity may be compromised.

The emergence of siRNAs and ASOs as a new drug modality now creates a significant manufacturing challenge (Tedebark et al. Methods mol. Biol. 2011, 683, 505; Kosuri, nat. Methods 2014, 11, 499). Current synthetic methods typically utilize the four-step solid phase phosphoramidite approach developed in the 1980s (Beaucage et al. Tetrahedron lett. 1981, 22, 1859). This approach can be easily automated and has been adapted for the synthesis of modified DNA structures. Unfortunately this strategy is not suitable for large scale synthesis (>100 kg) and is currently restricted to <10 kg batches of oligomeric product (Kim et al. Org. Process res. Dev. 2016, 20, 1439). Kilo scale synthesis is currently performed using columns up to 1 m in diameter with shallow beds (10-15 cm). Increasing the column size results in non-linear flow rates and reduced product purity meaning it is not possible to intensify the synthesis, and instead multiple parallel reactors are required. Reagents used in such methods are environmentally hazardous and present handling and disposal issues.

At present there is no reasonable method of in-line reaction monitoring which necessitates the use of large excesses of expensive phosphoramidites. These monomers contain multiple protecting groups (including 4′4-dimethoxytrityl protected 5′-OHs, benzoyl and isopropyl protected bases and 2-cyanoethyl protected phosphites) which further compromise atom efficiency and lead to the formation of by-products which must be separated during synthesis. The process uses prohibitively large volumes of acetonitrile (1000 Kg per Kg of oligonucleotide) and chromatographic purification of the final product is required to remove impurities arising from depurinations, base modifications (resulting from the formation of cyanoethyl adducts) and truncated sequences. Due to limitations of existing synthetic methods, current marketed RNA-based therapeutics are typically supplied with ˜90% purity and as complex mixtures of diastereoisomers (resulting from phosphorothioate modifications) and in fact there remains very limited understanding of the bioactivity of individual stereoisomers. Despite the recent development of a number of alternative methods, including the use of P(V) reagents (Knouse et al. Science 2018, 361, 1234) and covalent linkage of nucleotide building blocks to deoxynucleotidyl transferases (Palluk et al. Nat. Biotechnol. 2018, 36, 645) existing strategies share the same fundamental approach using sequential coupling and deprotection reactions on solid supports.

There is therefore a need for an improved method for manufacturing oligonucleotides, in particular therapeutic oligonucleotides such as ASOs and miRNAs.

### BRIEF SUMMARY OF THE DISCLOSURE

The present invention relates to a method of synthesising an oligonucleotide, for example a therapeutic oligonucleotide. The present invention aims to provide a scalable strategy for the manufacture of high purity oligonucleotides at low cost. In this manner, the present invention establish oligonucleotides as a viable treatment for disease. The present invention provides a method in which extension of the primer and cleavage of the extended primer to release the oligonucleotide, are concurrent. The primer-template can be used multiple times during a reaction, or incubation, period, thereby catalysing the oligonucleotide synthesis reaction,

In a first aspect, the present invention provides a method for producing a single stranded oligonucleotide, the method comprising:
i) providing a primer-template comprising a) a primer for initiation of oligonucleotide synthesis, b) a template which directs synthesis of an oligonucleotide product, and c) a cleavable site to enable release of the oligonucleotide product from the template;
ii) incubating the primer-template with a nucleic acid polymerase, one or more dNTPs, and a cleaving agent, to form a reaction mixture;
iii) maintaining the reaction mixture under conditions to allow for a) extension of the primer by the polymerase to form an extended primer-template and b) cleavage of the extended primer-template at the cleavable site by the cleaving agent to provide an oligonucleotide product;
iv) optionally separating the oligonucleotide product from the reaction mixture.

In a second aspect, the present invention provides an oligonucleotide produced by a method of the first aspect.

In a third aspect, the present invention provides a kit for producing a single stranded oligonucleotide, the kit comprising:
i. a primer-template comprising a) a primer for initiation of oligonucleotide synthesis, b) a template which directs synthesis of an oligonucleotide product, and c) a cleavable site to enable release of the oligonucleotide product from the template; and
ii. a nucleic acid polymerase and optionally one or more nucleotides
iii. a cleaving agent.

In a fourth aspect the present invention provides a support for producing a single stranded oligonucleotide, comprising immobilised thereon a population of primer-templates, wherein each primer-template comprises a) a primer for initiation of oligonucleotide synthesis, b) a template which directs synthesis of an oligonucleotide product, and c) a cleavable site to enable release of the oligonucleotide product from the template.

In a fifth aspect, there is provided a method of producing population of single stranded oligonucleotides, the method comprising:
i) providing a primer-template comprising a) a primer for initiation of oligonucleotide synthesis, b) a template which directs synthesis of an oligonucleotide product, and c) a cleavable site to enable release of the oligonucleotide product from the template;
ii) incubating the primer-template with a nucleic acid polymerase, one or more dNTPs, and a cleaving agent to form a reaction mixture;
iii) maintaining the reaction mixture under conditions to allow for a) extension of the primer by the polymerase to form an extended primer-template, and b) cleavage of the extended primer-template at the cleavable site by the cleaving agent to provide an oligonucleotide product;
iv) optionally separating the oligonucleotide product from the reaction mixture.
v) maintaining the reaction mixture in step iii) for a suitable reaction period to generate a population of oligonucleotides,
   wherein the nucleotides include modified thiotriphosphates (NTPaS) and wherein the oligonucleotide population comprises substantially the same stereoisomer;
   and wherein the polymerase is selected from the group consisting of polymerase from Thermococcus kodakaraensis (KOD), Stoffel, family B polymerase 9° N, Thermus filiformis (TfPol) and Marinithermus hydrothermalis (MhPol); Klenow fragment from E.coli, T4 and T7 polymerase, SFP1, Stoffel variant SF4-6, Stoffel homologs from Thermus filiformis and Marinithermus hydrothermalis (Mhpol) as shown in Table 3, or a variant of any of the afore-mentioned polymerases as shown in Table 3.

Also provided in an aspect of the invention is a reaction mixture for producing a single stranded oligonucleotide, the reaction mixture comprising a primer-template which comprises a) a primer for initiation of oligonucleotide synthesis, b) a template which directs synthesis of an oligonucleotide product, and c) a cleavable site to enable release of the oligonucleotide product from the template; and:
i) a nucleic acid polymerase and optionally one or more nucleotides
ii) a cleaving agent
iii) an oligonucleotide product, which is complementary to the template sequence.

Also provided is a cell for producing a single stranded oligonucleotide, comprising a primer-template which comprises a) a primer for initiation of oligonucleotide synthesis, b) a template which directs synthesis of an oligonucleotide product, and c) a cleavable site to enable release of the oligonucleotide product from the template.

### BRIEF DESCRIPTION OF THE DRAWINGS

Embodiments of the invention are further described hereinafter with reference to the accompanying drawings, in which:

In the figures and the Examples, T refers to a template sequence, E refers to an extended template, and P refers to a product. The sequences of the T, E and P sequences referred to herein are found in Table 1.

FIG. 1 shows oligonucleotide chemical modifications.

FIG. 2 shows Spinraza nucleotides and sequence (*=PS m=2′MOE).

FIG. 3 is a schematic showing scalable oligonucleotide manufacture according to the invention.

FIG. 4 shows: A) KOD (Merck, 2.5 Units) catalysed extension of T1 (Table 1, 20 μM) using dNTPs (1 mM) provides extended product E1 (Table 1, 7.0 min, expected mass 18170.8) in >99% yield; B) TmEndoV (ThermoFisher, 5 Units) catalyzed cleavage of extended template E1 (20 μM) provides product P1 (Table 1, 4.85 min, expected mass 5544.6) and template T1 (6.54 min, expected mass 12644.2) in >99% yield. The UV traces are shown on the left and the mass spectra are shown on the right; C) One-pot KOD and TmEndoV cascade process using T1 (20 μM) and dNTPs (0.25 mM each) provides product P1 (5.45 min, expected mass 5544.6).

FIG. 5 Polymerase and endonuclease activity at varying dNTP concentrations. PAGE analysis showing A) KOD (0.2 μM) catalysed T1 (20 μM) extension up to dNTP concentrations of 30 mM. B) TmEndoV (2 μM) catalysed cleavage of E1 (20 μM) up to dNTP concentrations of 2 mM C) TnEndoV (2 μM) catalysed cleavage of E1 (20 μM) up to dNTP concentrations of 20 mM.

FIG. 6 shows temperature optimization experiments A) Thermal shift assay showing the melting temperatures of TnEndoV (top peak), TmEndoV (middle peak) and PtEndoV (lowest peak); B) Polyacrylamide gel analysis of one-pot reactions catalysed by KOD and TnEndoV at varying temperatures. Lane 1: ladder, Lane 2: E1 standard (top band), T1 standard (middle band) and P1 (bottom band), Lane 3-8: One pot reactions performed at 60° C., 65° C., 70° C., 75° C., 80° C., 85° C. (from left to right); C) Overlay of HPLC traces showing KOD activity is unaffected by 24 hours pre-incubation at 70° C. Biotransformations were performed using T1 (20 μM), dNTPs (4 mM) and either fresh KOD (0.2 μM, green) or KOD pre-incubated for 24 hours (0.2 μM, blue) at 70° C. HPLC traces show the conversion to E1 following 30 minutes reaction time. D) Overlay of HPLC traces showing TnEndoV retains 85% activity following 24 hours pre-incubation at 70° C. Biotransfromations were performed using E1 (20 μM), and either fresh TnEndoV (2 μM, green) or TnEndoV pre-incubated for 24 hours (0.2 μM, blue) at 70° C. HPLC traces show the conversion to T1 and P1 following 30 minutes reaction time.

FIG. 7 shows the results of investigating polymerase/endonuclease activity towards self-priming templates with varying sequences. PAGE analysis showing P1 formation following cycles of polymerase catalyzed template extension and endonuclease catalyzed product cleavage. One-pot reactions were performed using KOD (0.2 μM), TnEndoV (2 μM), template (10 μM) and dNTPs (1 mM each). A DNA ladder is shown in lane 1, and T1 & E1 standards are shown in Lane 2. The yield of product P1 was largely unaffected by 5′-biotinylation (lane 4), alternative hairpin sequences (lane 7), varying the nucleobase pairing to inosine (Lane 8 & 9) or using a template and separate primer (lane 6). Placing a biotin at the hairpin (lane 5) or varying the length of the hairpin (Lane 10 & 11) led to a slight reduction in product yield.

FIG. 8 HPLC analysis of polymerase/endonuclease catalyzed P1 formation under optimized conditions. HPLC trace showing product P1 formed during the KOD (2 μM) and TnEndoV (2 μM) catalysed reaction using T1 (1 μM), dNTPs (4 mM each), in 20 mM Tris-HCI (pH 8), 20 mM MgCI₂, 100 mM arginine-HCI (pH 8), 100 mM glutamate-KOH (pH 8), DTT (10 mM), formamide (10%), trehalose (0.2 M), 1,2-propanediol (1 M) and acetylated BSA (0.01 mg/ml) following 12 hours incubation at 70° C. Product was generated following 330 cycles of template extension and product cleavage to afford 0.33 mM (equivalent to 1.9 g/L) of P1.

FIG. 9 shows polymerase activity towards modified dNTPs. Polymerase activity towards modified nucleotide triphosphates was evaluated using a time-resolved FRET based assay. Reactions were performed using three natural dNTPs and one modified NTP. The values for each polymerase are reported relative to the activity with natural dNTP and represent the average of measurements made in triplicate.

FIG. 10 Stereoselective synthesis of oligonucleotides containing a single phosphorothioate linkage. HPLC traces showing product formed during a one-pot polymerase-endonuclease reaction. Reactions were performed using T0-T13 (10 μM) and a mixture of 3 unmodified NTPs (1.4 mM each) and A) Sₚ-dATPaS; B) Sₚ-dCTPas; C) Sₚ-dTTPaS; D) Sₚ-dGTPaS; E) Rₚ/Sₚ-dATPaS; F) Rₚ/Sₚ-dCTPaS; G) Rₚ/Sₚ-dTTPaS; H) Rₚ/Sₚ-dGTPaS; (0.7 mM). Comparison of the enzymatically synthesized products to diastereomeric mixtures produced by chemical synthesis shows that the biocatalytic process affords products as single diastereomers in >99% d.e.

FIG. 11 LC-MS analysis of TnEndoV activity towards modified oligonucleotides. UV traces showing products formed during TnEndoV (2 μM) catalysed cleavage reactions of extended templates (20 μM) A) E6; B) E2; C) E3; D) E4; E) E5. The deconvoluted mass for each 18 mer product and the template is provided in the tables. EndoV cleavage of the phosphorothioate modified oligonucleotide E6 provided the expected product (P_{A}) and a 5′-dephosphorylated analogue (P_{B}). Incubating a P_{A} standard (20 μM) in the reaction buffer at 70° C. leads to 5′-dephoshorylation confirming that the side reaction is not enzyme catalysed.

FIG. 12: Substrate scope of the EndoV panel. PAGE analysis showing EndoV (2 μM) catalyzed cleavage of extended templates (20 μM) containing no modifications (E1), 18 consecutive phosphorothioate (E6), 2′-fluoro (E2), 2′-methoxy (E3) or 2′-methoxyethoxy (E4) modifications or one LNA modification (E8) 3′-of the cleavage site. Samples were analyzed following 2 and 4 hours incubation at 70° C. The standards comprise of extended template E1, template T1 and a 18 mer oligonucleotide (P).

FIG. 13: preparative scale synthesis of P1. HPLC traces showing P1 synthesis catalysed by KOD (2 μM) and TnEndoV (2 μM) using T2 (4 μM) and dNTPs (4 mM each). A) HPLC of the reaction mixture following incubation for 12 hours B) Isolated product. The table shows the observed masses and impurities present in the crude reaction mixture and isolated product. Sequences printed in bold correspond to the target product.

FIG. 14: Substrate scope of the polymerase panel. PAGE analysis showing polymerase (0.2 μM) activity towards modified NTPs. Reactions were performed using T22 (20 μM) and a mixture of 3 unmodified NTPs (0.25 mM each) and one modified NTP (0.25 mM) an incubated at 70° C. for 1 hour. Polymerases able to transcribe the template and incorporate two copies of a modified NTP result in full length extension. Control reactions were performed in the absence of modified NTP to determine the rate of background misincorporation of non-complementary bases. A) Polymerase activity towards dNTPs (PO), Sₚ-dNTPaS (Sₚ), Sₚ-2′-fluoro-NTPaS (Sₚ-2′F) Rₚ-dNTPaS (Rₚ), 2′fluoro-NTPs (2′F) and LNA-NTPs (LNA). B) polymerase activity towards 2′-methoxy modified NTPs where + indicates 2′MeO-NTP addition and − indicates control reactions in the absence of 2′-MeO-NTP. T22=5′-CTG ACT GAC ACG TGC ACC ATT GGT GCA CGI G-3′

FIG. 15 Polymerase activity towards sequential incorporation of 2′MeO-NTPs and LNA-NTPs. Templates (T18-T21, 20 μM) encoding eight copies of a single nucleobase, were extended using the complementary 2′MeO-NTP (1 mM) or LNA-NTP (1 mM) and the most suitable polymerase, SF4-6 (0.2 μM) or KOD DGLNK (0.2 μM), respectively. LC-MS analysis revealed reactions gave rise to mixtures of partially extended products after 12 hours incubation. The peaks labelled in the UV spectra were assigned to truncated products by MS analysis, additional peaks could not be assigned. nt additions=number of nucleotide bases incorporated by the polymerase.

FIG. 16: Polymerase catalysed reactions using modified NTP mixes. Template T2 (20 μM) was extended using mixtures of Sₚ-dNTPaS (2, 0.25 mM each) and 2′F-NTPs (3, 0.25 mM each) using KOD (0.2 M) and TfPol* (0.2 μM) respectively. Template T7 (20 μM) was extended using mixtures of Sₚ-2′F-A/GTPaS (4, 0.25 mM each) and KOD (0.2 μM). PAGE analysis showed that reactions had reached full conversion to the extended product; B) LC-MS analysis was used to confirm the product identity. No. additions=number of nucleotide bases incorporated by the polymerase.

FIG. 17: TnEndoV stereoselectivity at the phosphorothioate centre undergoing hydrolysis. Stereodefined extended templates were produced using KOD (0.2 μM) and Sₚ-dNTPaS (0.5 mM each). TnEndoV (2 μM) mediated cleavage of the enzymatically synthesized products (20 μM) was compared to the cleavage of the corresponding stereo-random oligonucleotides (20 μM). PAGE analysis shows A) Polymerase catalyzed extension of templates T1, and T15-T17 (20 μM) proceeds to completion. Each template encodes an 18 mer oligonucleotide product with a different nucleobase at the first position. B) TnEndoV (2 μM) mediated cleavage of stereodefined oligonucleotides with Rₚ-linkages (lane 9-12) procceds to completion, whilst the cleavage of the corresponding stereorandom oligonucleotides stalls at 50% conversion (lane 13-16). Activity is unaffected by the base 3′ of the cleavage site.

FIG. 18: One-pot polymerase/EndoV catalyzed production of P19-P32. UPLC chromatograms (recorded at 260 nm) show product formation following a one-pot polymerase-endonuclease catalyzed reaction. Specific reaction conditions for each biotransformation are given in Table S1. Product and side-product masses were confirmed by mass spectrometry. T: template, E: extended template. Nucleotide sequence printed in bold corresponds to the correct 8 mer product (+: LNA, *: phosphorothioate linkage f: 2′-fluoro, P: 5′ phosphate group, HO/OH: 5′/3′ hydroxyl group). Sequences printed in bold correspond to the correct product. Bases highlighted in red correspond to non-templated overextension.

FIG. 19: Preparative scale synthesis of Vitravene. UPLC chromatograms (recorded at 260 nm) showing A) the crude reaction mixture following incubation at 70° C. for 12 hours. B) The isolated product following reaction work-up. During the reaction the product is 5′-dethiophosphorylated (peak a). Incubating a standard of the product in reaction buffer leads to 5′-dethiophosphorylation confirming that the side reaction is not enzyme catalysed. The target product contains a 5′hydroxyl, so any remaining 5′thiphosphate groups were removed during the reaction work-up using an alkaline phosphatase.

FIG. 20: Polymerase activity towards Rp-dNTPsaS in the presence of cobalt and manganese cofactors. A) PAGE analysis showing KOD catalyzed extension of templates encoding eight copies of a single nucleobase, using the complementary Rp-dNTPaS (1 mM) and in the presence of 0.5 mM CoCI₂ or MgCI₂. B) LC-MS analysis showing the addition of eight Rp-dATPaS nucleotides to a self-priming template catalyzed by KOD in the presence of a mixture of 1 mM CoCI₂ and 0.4 mM MnCI₂. The peak at 5.2 minutes corresponds to the extended product (observed mass 12141) and the peak at 0.5 minutes corresponds to unreacted NTPs.

FIG. 21: Endonuclease activity in the presence of alternative nucleophiles. UV traces following LC-MS analysis of EndoV reactions performed in the presence of high concentrations of different nucleophiles. Nucleophilic attack of the extended template E1 yielded ‘modified 18 mer products’ containing glycerol (mass detected 5618.8), ethyleneglycol (mass detected 5587.4), 1,2-propandiol (5601.9) or 1,3-diamino propanol (mass detected 5616.6) functionalized 5′-phosphate groups. The major product was the canonical 18 mer with an unmodified 5′-phosphate group.

FIG. 22: Biocatalytic synthesis of oligonucleotide sequences. Oligonucleotides with varying sequence and chemical modifications were produced in one-pot biotransformations using the most suitable polymerase and TnEndoV. The percentage conversion describes the conversion of NTP starting material to product. For specific reaction conditions see Table 3.

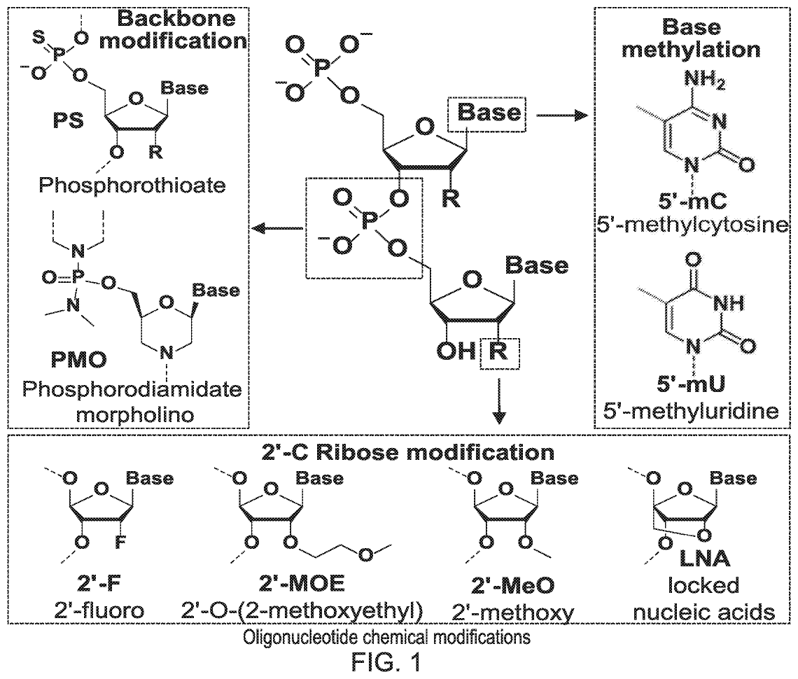

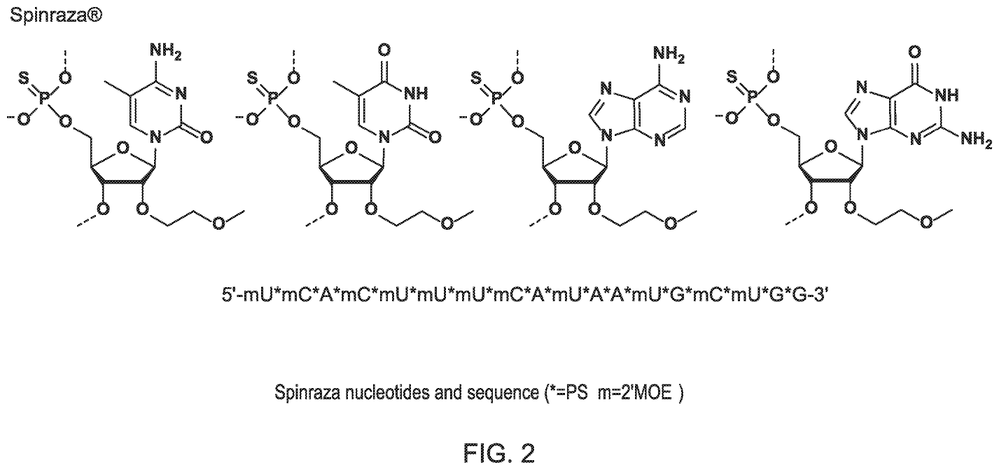

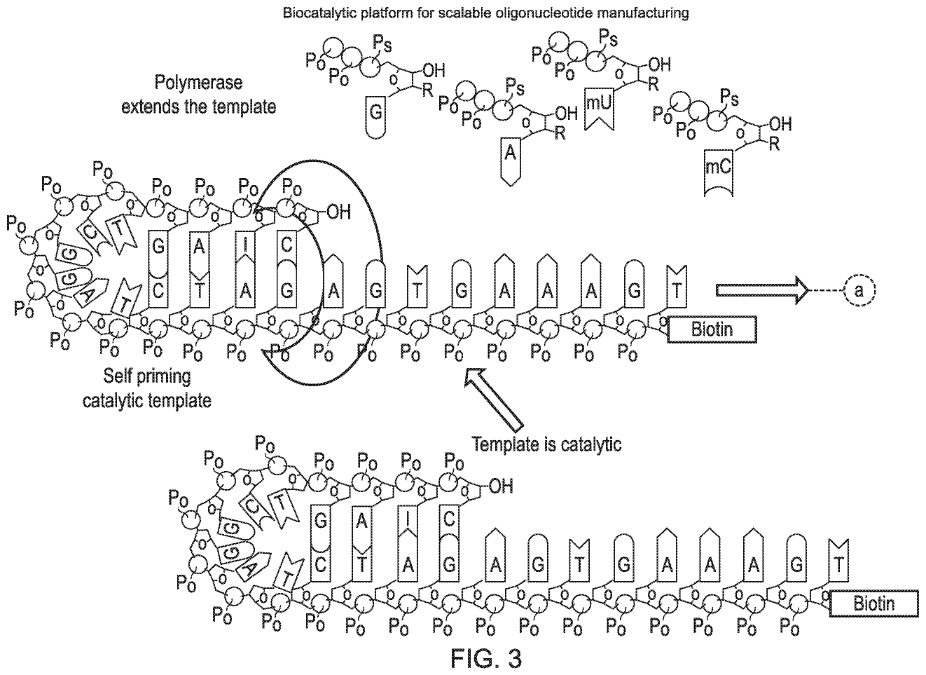

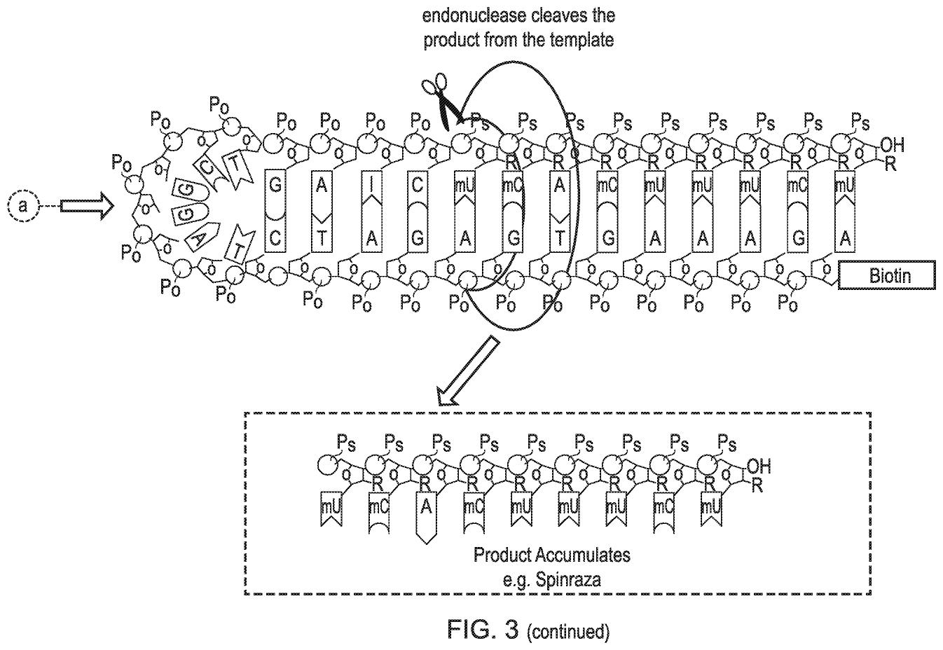

### DETAILED DESCRIPTION

The present invention provides a method for synthesising a single stranded oligonucleotide, for example a therapeutic oligonucleotide, in a manner which is versatile and scalable.

The invention is based upon the novel concept of providing a recyclable, or catalytic, primer-template, which is extended by a polymerase to form a duplex nucleic acid molecule, referred to herein as the extended primer template. A suitable cleaving system is used to cut a strand of the duplex to allow release of the extended portion, which is the desired single stranded oligonucleotide product. Suitably, the duplex is cut at a cleavable site built into the nucleic acid molecule, thereby releasing a single stranded oligonucleotide product and regenerating the primer-template. The cleavage to regenerate the primer-template enables repeated cycles of extension and cleavage, which will result in accumulation of the oligonucleotide product in the reaction mixture. A method of the invention is therefore able to deliver a target oligonucleotide in a single operation, which contrasts with the iterative rounds of chain extension, oxidation and deprotection associated with chemical synthesis of oligonucleotides known in the art. In chemical synthesis, numerous iterative reactions are required to produce a short oligomer. In the present invention, an oligonucleotide can be produced in a single reaction. The method of the invention has the advantage of being capable of being performed under aqueous conditions, without the requirement for large volumes of acetonitrile. Furthermore, the method of the invention is more atom efficient than standard phosphoramidite chemistry as protecting groups are not required. The method of the invention also has the advantage of enabling the delivery of oligonucleotide products with significantly higher purity compared to those produced via chemical synthesis, thus alleviating the need for costly chromatographic purifications. In addition, the method of the invention may be isothermal. This has the advantage of reducing the energy required to change the temperature during different parts of each cycle.

The present invention provides a method for the manufacture of therapeutic oligonucleotides in a scalable manner, by providing a method for production of an oligonucleotide which may comprise one or more modified, or non-natural, nucleotides, at high levels of purity and in a scalable manner to enable production of therapeutic oligonucleotides in large amounts. The method of the invention may utilise a modified polymerase enzyme suited to incorporate one or more modified nucleotides into an nucleotide chain.

The invention is based upon the use of a primer-template, a polymerase and a cleaving agent, concurrently, to produce an oligonucleotide, wherein after release of the newly synthesised oligonucleotide from the template, the primer-template is available in the same reaction for one or more further rounds of oligonucleotide synthesis. As the template is copied, the synthesised oligonucleotide is cleaved by the cleaving system and released from the template, leaving the primer-template available for a further round of oligonucleotide synthesis. Thus, the combined use of the primer-template, polymerase and cleaving agent in the same reaction means that the reaction is not stoichiometric, and enables the generation of product in an amount which exceeds the amount of template present in the reaction.

### Definitions

Herein, a “nucleic acid molecule” refers to a chain of nucleotides having a particular sequence. A nucleic acid molecule may contain all four nucleotides (G, A, T/U and C) or any combination of the same (e.g., G, A, T/U or C, or any combination thereof).

An “oligonucleotide” is a short nucleic acid sequence, typically 5 to 100 nucleotides in length.

Herein, “single stranded nucleic acid” refers to a first multimer of nucleotides which is not bound (or base paired) to a second, separate, multimer of nucleotides with which it is capable of forming a duplex. A “double stranded nucleic acid” sequence is a first multimer of nucleotides which is based paired with a second, separate multimer of nucleotides to form a duplex.

As used herein, the term “oligonucleotide synthesis reaction” refers to a reaction in which oligonucleotides are synthesized by adding monomers one by one onto a growing chain. The nucleotides may be added to the 3′ end of the growing chain. The reaction may be performed by a suitable polymerase.

References herein to a “nucleoside triphosphate” refers to a molecule containing a nucleoside (i.e. a base attached to a deoxyribose or ribose sugar molecule) bound to three phosphate groups. A ‘nucleotide’ or ‘nucleic acid residue’ refers to to a molecule containing a nucleoside bound to one phosphate. A nucleotide refers to any compound and/or substance that is or can be incorporated into an oligonucleotide chain, typically via a phosphodiester linkage. Examples of nucleoside triphosphates that contain deoxyribose are: deoxyadenosine triphosphate (dATP), deoxyguanosine triphosphate (dGTP), deoxycytidine triphosphate (dCTP) or deoxythymidine triphosphate (dTTP). Examples of nucleoside triphosphates that contain ribose are: adenosine triphosphate (ATP), guanosine triphosphate (GTP), cytidine triphosphate (CTP) or uridine triphosphate (UTP). Other types of nucleosides may be bound to three phosphates to form nucleoside triphosphates (nucleotides), such as naturally occurring modified nucleosides and artificial nucleosides.

A “modified” nucleic acid is one in which the phosphodiester linkage, the sugar, and/or base has been altered.

“RNA” is a linear molecule composed of four types of smaller molecules called ribonucleotide bases: adenine (A), cytosine (C), guanine (G), and uracil (U). “DNA” is a linear molecule composed of four types of smaller molecules called deoxyribonucleotide bases: adenine (A), cytosine (C), guanine (G), and thymine (T). RNA and DNA may each be single stranded or double stranded. RNA and DNA may each form secondary structures.

“MicroRNA” is a single stranded RNA of about 17-25 nucleotides, which may have a role in cell proliferation, apoptosis or differentiation. A miRNA may comprise a modified nucleotide. miRNA may be a therapeutic oligonucleotide.

An “ASO” (anti-sense oligonucleotide) is a short, synthetic DNA or RNA which may be complementary to a mRNA target. An ASO may down-regulate a downstream target. An ASO is typically modified, for example phosphorothioated.

An “aptamer” is a nucleic acid molecule which binds to a target such as a protein, nucleic acid, oligosaccharide, small molecule, hormone, cytokine, signalling molecule, metal ion or organic molecule, with high affinity and specificity. An aptamer may be single stranded or double stranded, or partially single stranded or partially double stranded. An aptamer may comprise a modified nucleotide.

A “polymerase” catalyses the formation of oligonucleotide chains through the addition of successive nucleotides derived from nucleoside triphosphates or deoxynucleoside triphosphates. The polymerase reaction takes place only in the presence of an appropriate nucleic acid template. Each incoming nucleoside triphosphate first forms an appropriate base pair with a base in this template. The polymerase then links the incoming base with the predecessor in the chain. Thus, polymerases are template-directed enzymes.

A “primer”, as used herein, refers to an oligonucleotide, either natural or synthetic, that is capable of acting as a point of initiation of nucleic acid synthesis and being extended from its 3′ end along a template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually primers are extended by a DNA polymerase.

Herein, a “template” is the nucleic acid sequence which is copied by the polymerase. The template provides the complementary sequence of the desired oligonucleotide to be synthesised.

An “extended primer-template” is the product formed during oligonucleotide synthesis, where the primer initiates synthesis of a nucleic acid chain which is base paired to the template strand.

A “primer-template” is a template which comprises a primer, and therefore does not require a separate primer to initiate oligonucleotide synthesis.

A “self-priming template” is a nucleic acid molecule which comprises a sequence allowing self-binding to form a double stranded primer sequence and a single stranded template sequence.

A “hairpin loop” or a “stem loop” structure is an intramolecular base pairing pattern that can occur in single-stranded DNA or RNA. A hairpin loop occurs when two regions of the same strand, usually complementary in sequence when read in opposite directions, base-pair to form a double helix that ends in an unpaired loop.

“Cleavage” refers to the cutting of the phosphodiester bond between two nucleic acid residues in a polynucleotide chain.

A “cleavable site” as referred to herein is the bond, or pair of bonds between nucleotides in a nucleic acid molecule which can be cut to break the nucleic acid molecule or to release one strand of the nucleic acid molecule. A cleavable site can be defined by a particular nucleotide sequence that is capable of being recognized by an enzyme. A cleavable site can also be defined by the position of modified nucleotides that are capable of being recognized by certain nucleases.

A “cleavage” enzyme or system as referred to herein is capable of cleaving the phosphodiester bond within a polynucleotide chain.

“Extension” refers to the synthesis of a new oligonucleotide strand, suitably by the action of a polymerase.

“Immobilisation”, herein refers to reversible or non-reversible attachment of a nucleic acid molecule or binding partner to a support.

A sequence which is “complementary” to another sequence has a nucleic acid sequence which base pairs with the nucleotides of the other sequence, to form a stable duplex or double stranded sequence. By “substantially complementary” is mean that not all of the base pairs between the two sequences match, but there is sufficient base pairing to form a stable duplex.

Herein, “release” refers to dissociation of the synthesised oligonucleotide strand from the template.

A “kit” refers to any delivery system for delivering materials or reagents for carrying out a method of the invention.

### Reagents

The present invention utilises, in the same reaction, reagents for the synthesis of a desired oligonucleotide. The reagents present in the same reaction may include a primer, a template, a polymerase, a cleaving system, nucleotide residues, co-factors and reagents for reaction optimisation.

### I) The Primer-Template

A primer-template may be RNA or may be DNA, and may include modified RNA or DNA.

A primer-template comprises features which enable it to perform three functions-as a primer to initiate polymerase mediated extension; as a template for synthesis of a new oligonucleotide strand; and to enable cleavage of the new oligonucleotide from the nucleic acid molecule. It may therefore comprise a primer for initiation of oligonucleotide synthesis, a template which directs synthesis of the oligonucleotide, and a cleavable site to enable release of the oligonucleotide product from the template.

The primer-template directs extension by sequential addition of nucleotides to the 3′ end of the primer, to form an extended primer-template. The primer is positioned with respect to the template sequence so that the template sequence, preferably in full, is copied in extension of the primer. The primer sequence may be positioned upstream of the template sequence. The primer sequence may be a sequence which is bound to a sequence upstream of the template sequence, to form a double stranded (duplex) sequence upstream of the single stranded template sequence, and from which polymerase activity is initiated. Therefore primer-template comprises a single stranded nucleic acid template sequence, and a double stranded (or duplex) portion comprising the primer sequence. The single stranded template sequence therefore extends beyond the double stranded portion comprising the primer, to form a 5′ tail, or sticky end, which is available to be copied by extension of the 3′ end of the primer sequence. The template sequence may be a 5′ to 3′ sequence extending from the double stranded portion.

The double stranded portion of the primer-template which comprises the primer is formed from a pair of complementary nucleic acid sequences. The double stranded portion comprising the primer may be formed from a single stranded nucleic acid molecule, comprising a pair of complementary sequences which bind such that the single stranded nucleic acid molecule is folded to form a secondary structure. A primer template formed from a single stranded nucleic acid molecule comprising a pair of complementary sequences may be referred to herein as a self-priming template. The pair of complementary sequences may be provided in the single stranded nucleic acid molecule such that when bound to form a duplex, the nucleic acid molecule is folded on itself, asymmetrically, to form a double stranded portion with a fold or loop at one end, and an overhang of single stranded sequence at the other end which forms the template. Suitably, a single stranded molecule may be folded asymmetrically such that the end of the overhanging, single stranded template sequence is the 5′ end, and the end of the sequence which forms the double stranded portion is the 3′ end. This structure provides a free 3′ OH end for extension, to form the new oligonucleotide sequence, by base pairing to the template sequence. Suitably, the single stranded sequence is folded such that the 3′ terminal nucleotide is base paired with the nucleotide immediately adjacent (upstream) to the first nucleotide of the template sequence. In this manner, the single stranded overhang starts with the template sequence to be copied.

Suitably, the secondary structure formed by pairing of the complementary sequences of the nucleic acid molecule, is a hairpin loop. The hairpin loop is formed by a single stranded nucleic acid molecule adopting a secondary structure in which the sequence is folded back upon itself to form a double stranded stem portion and a loop at one end. The hairpin loop single stranded nucleic acid molecule assumes a hairpin structure due to the presence of the pair of complementary sequences separated by a third sequence, such that the complementary sequences base pair to form a double stranded stem portion, and the third intervening sequence forms with a loop at one end of the double stranded stem portion. The 3′ end of one of the pair of complementary sequences serves as a primer for transcription of a nucleic acid sequence. Suitably, the length of the stem portion and/or the size of the loop portion are of a suitable length of size to allow for a polymerase to bind to the hairpin loop and initiate transcription from the free 3′ hydroxyl group at one end of the stem portion.

The length of the stem portion is determined by the length of the complementary sequences in the single stranded sequence, which base pair to form the hairpin loop. The size of the loop is typically be determined by the length of non-complementary sequence between the two sections of complementary sequence which base pair to form the stem portion. A hairpin loop comprises a 5′ end and a 3′ end, typically at the ends of the double stranded stem portion. The 3′ end comprises a free OH group to which a polymerase can initiate primer extension. The hairpin loop may be any suitable size which allows it to serve as a primer for transcription. Suitably, the loop portion of the hairpin comprises more than 3 nucleotides, to allow the loop structure to form. Suitably, the loop portion of the hairpin loop comprises more than 4 nucleotides, suitably 4 to 30 nucleotides, more suitable 4 to 25, 4 to 20 or 10 to 20 nucleotides, or any range or integer in between. The double stranded stem portion may be any suitable length, for example 1 to 200 nucleotides in length, or 1 to 190, 1 to 180, 1 to 170, 1 to 150, 1 to 120, 1 to 100, or 10 to 90, 20 to 80, or 30 to 50 nucleotides in length, or any range using any of the aforementioned start and end points, or any length therebetween. A most suitable length for the stem portion of the hairpin loop may be 10 to 30 nucleotides in length, most suitably 10 to 20 nucleotides in length.

The stem portion of a hairpin loop is typically double stranded, formed from a single strand of nucleic acid folded back on itself as described herein. The stem portion of a hairpin loop has a free 5′ end and a free 3′ end. The two strands of the stem portion may be the same length, thereby forming a blunt end, or may be different lengths, thereby forming a sticky end in one strand. The presence of a blunt end or sticky end will be determined by the location of the complementary sequences within the single stranded nucleic acid sequence which forms the hairpin loop. Suitably, the stem portion comprises a blunt end. The nucleic acid molecule comprising the template sequence which extends beyond the double stranded stem portion (for the polymerase to copy) may be referred to as having a sticky end, or a sequence without any complementary bases bound thereto.

Examples of complementary sequences for provision in a single stranded nucleic acid molecule to enable formation of a hairpin loop are shown in Table 1. Examples of non-complementary sequences which form a loop structure are shown in Table 1. A single stranded nucleic acid sequence to form a hairpin loop for use in the present invention may comprise any combination of complementary (stem) sequences and loop sequences as shown in Table 1.

Alternatively, the primer-template may comprise a separate primer and template, which are not formed from a single self-complementary nucleic acid sequence. Instead, the primer-template may comprise i) a single stranded nucleic acid molecule template; ii) a separate primer sequence base paired thereto; and iii) a cleavable site. Suitably, the primer sequence is shorter than the single stranded molecule template. Suitably, the primer is base paired at the 3′ end of the single stranded nucleic acid molecule template, thereby providing a single stranded template extending downstream of a duplex formed by the primer and single stranded nucleic acid molecule template. Suitably, the primer comprises a free 3′hydroxyl group for extension by the polymerase. Therefore, such a primer-template may be equivalent to the self-priming template as described herein, but without the secondary structure such as the hairpin loop.

The primer and template may be linked by other means in addition to base pairing. For example, the primer may be immobilised on the template, or the primer and template may be chemically linked. Any suitable means may be used to bind the primer to the template. If the primer and template separate during the reaction, it may result in generation of by product rather than the desired oligonucleotide, because the primer may be used to direct synthesis (i.e. the primer may be copied), in addition to or as well as the template. Use of means to bind the primer and template such that do separate during the reaction reduces the likelihood of by product formation, and aids downstream product isolation.

Where the primer part of the primer-template is provided as a separate nucleic acid molecule, it may be an RNA molecule or a DNA molecule. It may comprise one or more natural and/or modified nucleotide residues. A primer may be a 3′ ribonucleotide primer, comprising a DNA sequence with an ribonucleotide residue at its 3′ end. Such a primer retains its function after cleavage of the oligonucleotide from the template sequence, and can be re-used. Suitable methods will be known to a person skilled in the art for preparing a 3′ ribonucleotide primer.

The primer is suitably hybridised to the template prior to oligonucleotide synthesis. Where a self-priming template is used, the secondary structure is formed prior to oligonucleotide synthesis. The hybridisation conditions required to form the primer-template will depend on factors including the specificity of the pair of complementary primer sequences or the primer and template sequences.

The hybridisation of the primer and template may take place in the oligonucleotide synthesis reaction, or prior thereto. If hybridisation takes place in the synthesis reaction, in the presence of the polymerase enzyme, cleaving system and any other reagents, then the hybridisation conditions must be such that the do not affect the function of the other reagents.

The template portion of the primer-template comprises a single stranded sequence which provides the sequence to be copied, for synthesis of the desired oligonucleotide sequence. Suitably the sequence of the template is such that it does not anneal or base pair with any part of the primer-template, so that it remains single stranded and available for use as a template for polymerase mediated extension. Suitably therefore the sequence of the template portion of the primer-template is not sufficiently complementary any of the remainder of the sequence of the primer-template to allow duplex formation of the template. Therefore the template sequence comprise no more than 1, 3, 5, 10, 15 or 20% sequence complementarity to the remainder of the primer-template nucleic acid molecule. By “sequence complementarity” is meant that the sequences are capable for base pairing to form a double stranded sequence.

The template will comprise a sequence which is complementary to the sequence of the desired oligonucleotide to be synthesised. The template may therefore comprise a sequence which is complementary to a therapeutic oligonucleotide, such as an aptamer, mRNA, or siRNA. Suitably, the template sequence is 100%, 99%, 98%, 97%, 96%, 95%, 94%, 93%, 92%, 91%, 90%, 95% or 99% complementary to the oligonucleotide sequence to be synthesised.

The template may comprise two or more portions, wherein each portion directs the synthesis of an individual, or different, desired oligonucleotide. Therefore, a single template may be used for the production of two or more different oligonucleotides. The template sequence may therefore direct the synthesis of an oligonucleotide product, which may then be cleaved into the individual, desired oligonucleotides. The template sequence may suitable sequence to incorporate one or more restriction enzyme sites into the product, to allow the product to be cleaved into individual desired oligonucleotides.

The template may comprise sequence to direct synthesis of a feature or motif, for example without limitation a termination sequence, a spacer sequence, a polyadenylation sequence, a tag, and/or a cleavable site. Where a spacer or transcription termination sequence is included in the template, a cleavable site (or the complementary sequence thereof) may be included to enable cutting of the synthesised oligonucleotide to produce the desired product without any additional sequence.

The template sequence may suitably be the same length as the length of a desired oligonucleotide, or may be longer for example where it comprises a template sequence for two or more oligonucleotides, or a template for one or more features or motifs as described above.

A template may be any suitable length. A template may be 3 to 200 nucleotides in length, suitably 5 to 200, 5 to 150, 5 to 100, 10 to 100, 10 to 90, 10 to 80, 10 to 70, 10 to 60, 10 to 50, 20 to 50, 20 to 40 or 20 to 30 nucleotides in length, or any range using any of the aforementioned start and end points, or any length therebetween. A most suitable length for the template sequence may be 10 to 30 nucleotides in length, most suitably 10 to 20 nucleotides in length.

The extended primer template may be RNA, DNA or may be a hybrid of RNA and DNA. For example, where the primer-template is DNA, and the extended portion is RNA, the extended primer-template is a hybrid of RNA and DNA. The extended primer-template may comprise one or more modified nucleotides.

The primer-template comprises a cleavable site to enable release of the synthesised oligonucleotide from the template, and recycling of the primer-template for one or more additional rounds of oligonucleotide synthesis.

Any suitable cleavable site may be used.

A cleavable site is suitably positioned in the stem portion of the nucleic acid molecule, upstream of the primer initiation site. It may be positioned in the stem portion immediately adjacent to the primer initiation site, or may be within 1 to 5 nucleotides of the primer initiation site, suitably within 1 to 2 nucleotides of the primer initiation site, more suitably there may be a single residue between the cut site and the primer initiation site. The cleavable site is suitably selected such that cleavage does not alter either the oligonucleotide product or the primer-template. Put another way, aside from cutting the oligonucleotide from the primer-template, the cleavage is traceless and cannot be detected in the oligonucleotide product or in the primer-template from which the oligonucleotide is released.

A cleavable site may include cleavable linkages, cleavable nucleotides or a specific sequence motif.

Cleavable linkages include for example, a modified 3′-5′ internucleotide linkage in place of one of the phosphodiester groups, such as, dialkoxysilane, phosphorothioate, phosphorodithioates, methylphosphonates, mesyl phosphoramidites and phosphoramidate internucleotide linkage.

Cleavable nucleotides may comprise, without limitation, a deaminated base, a base analog, an abasic site or a urea site, or a mismatched nucleoside. The cleavable oligonucleotide analogs may also include a substituent on, or replacement of, one of the bases or sugars, such as 7-deazaguanosine, 5-methylcytosine, inosine, uridine, and the like. Cleavable nucleotides comprising base analogs cleavable by an endonuclease, suitably an endonuclease which leaves a free 3′ hydroxyl group to enable further rounds of extension. In some embodiments, cleavable nucleotides include nucleotides comprising base analogs cleavable by formamidopyrimidine DNA glycosylase which include, but are not limited to, 7,8-dihydro-8-oxoguanine, 7,8-dihydro-8-oxoinosine, 7,8-dihydro-8-oxoadenine, 7,8-dihydro-8-oxonebularine, 4,6-diamino-5-formamidopyrimidine, 2,6-diamino-4-hydroxy-5-formamidopyrimidine, 2,6-diamino-4-hydroxy-5-N-methylformamidopyrimidine, 5-hydroxycytosine, 5-hydroxyuracil. In some embodiments, cleavable nucleotides include nucleotides comprising base analogs cleavable by thymine DNA glycosylase which include, but are not limited to, 5-formylcytosine and 5-carboxycytosine. In some embodiments, cleavable nucleotides include nucleotides comprising base analogs cleavable by human alkyladenine DNA glycosylase which include, but are not limited to, 3-methyladenine, 3-methylguanine, 7-methylguanine, 7-(2-chloroehyl)-guanine, 7-(2-hydroxyethyl)-guanine, 7-(2-ethoxyethyl)-guanine, 1,2-bis-(7-guanyl)ethane, 1,N<6>-ethenoadenine, I,N<2>-ethenoguanine, N<2>,3-ethenoguanine, N<2>,3-ethanoguanine, 5-formyluracil, 5-hydroxymethyluracil, hypoxanthine. In some embodiments, cleavable nucleotides include 5-methylcytosine cleavable by 5-methylcytosine DNA glycosylase. be nucleotide analogs such as deoxyuridine or 8-oxo-deoxyguanosine that are recognized by specific glycosylases (e.g. uracil deoxyglycosylase followed by endonuclease VIII, and 8-oxoguanine DNA glycosylase, respectively). In some embodiments, cleavable nucleotides include nucleotides comprising base analogs recognisable by endonuclease V, for example inosine, uracil, hypoxanthine, xanthine, abasic sites, urea sites, base pair mismatches insertions and deletions. A suitable cleavable nucleotide is inosine, for cleavage by endonuclease V.

A cleavable sequence motif may be any sequence which is recognised by an enzyme which cuts the sequence in a sequence specific manner. For example a cleavable sequence may be a specific sequence recognised by an enzyme, for example an endonuclease. The cut may be upstream, downstream or within the sequence motif. By way of example, a sequence motif may be GAGTCNNNN*N (where N is any base and * is the cleavage site), which is recognised by Nt BstNBI; GGATCNNNN*N recognised by NtAlwl; or GCTCTTCN*N recognised by NtBspQI. Other examples will be known to persons skilled in the art. Suitably, the cut site (e.g. nucleotide) is positioned upstream and suitably within 1 to 5 nucleotides of the primer initiation site, suitably within 1 to 2 nucleotides of the polymerase initiation site, or immediately adjacent to the polymerase initiation site.

Suitably, a single cleavable site is provided in the nucleic acid molecule, such that the primer/template can be recycled after release of the oligonucleotide product. Suitably, the oligonucleotide product produced in a reaction does not comprise a cleavable site recognised by the cleaving system used in that reaction.

A cleavable site may be a deaminated base for endonuclease V cleavage, or the relevant sequence for a nicking endonuclease.

Suitably, deaminated bases are not used in a therapeutic oligonucleotide product, as they can Watson crick base pair to multiple bases and are therefore not specific.

A cleavable site may be a consensus sequence recognised by a restriction enzyme, for example an endonuclease enzyme. Where a cleavable site is an enzyme binding site, a complement of a cleavable site may be provided in the template portion, to generate a cleavable site during synthesis of the oligonucleotide molecule so that the cleavable site is provided in the newly generated sequence.

The primer-template may comprise one or more motifs or sequences, for example and without limitation, including a binding site, a label, an immobilisation site, and a cleavable site. One or more of any such motifs or sequences may be present in or operably linked to the primer sequence comprising a hairpin loop or the single stranded nucleic acid template, or both.

An immobilisation site, or moiety, may be provided to bind the nucleic acid molecule to a support, for example a solid support, for example in the formation of an array. An immobilisation site, or binding site, may be any suitable sequence or binding agent/moiety which mediates binding either directly to the support or to a binding partner provided on the support. Examples of binding partners include oligonucleotide adaptors configured to bind to the nucleic acid molecule, analyte/antibodies, oligonucleotide pairs, nucleic acid molecules and complementary sequences, aptamers, affinity binding proteins and receptors or binding proteins, lipids, carbohydrates, and the like with their binding partners. A suitable example is streptavidin, which may bind to a biotinylated nucleic acid molecule.

An immobilisation site may be provided either in the loop of the nucleic acid molecule, the stem, or downstream (5′) of the template sequence. Suitably, an immobilisation site is provided in a position such that immobilisation of the nucleic acid molecule does not sterically interfere with binding and/or activity of the polymerase or cleaving system to the nucleic acid molecule. In a suitable embodiment, an immobilisation site may be provided in the loop of the hairpin loop, suitably at the mid-point of the loop. In a suitable embodiment, an immobilisation site may be provided in the template sequence, provided that the presence of the immobilisation site does not affect the activity of the polymerase. In a suitable embodiment, an immobilisation site may be provided at or downstream to the 5′ end of the template sequence.

A primer and template may be designed using suitable techniques available in the field, for example Primer-BLAST and implemented using Primer 3, BLASTN, Melting, pandas and the Python standard library.

Parameters of primer and template design include the length and the melting temperature of the annealing sequences. For a given sequence, increasing the length increases the strength of the specific binding interaction, but may also increase inappropriate ligations by non-specific binding to off-target sequences and/or decrease the probe's effective concentration in the reaction.

The self-priming template may be prepared using any suitable method, for example enzymatic or chemical synthesis. A self-priming template may be prepared using recombinant or cloning methods, which will be known and available to a person skilled in the art. A suitable method may comprise enzymatic amplification of a nucleic acid to provide multiple copies of a primer-template.

### II) Nucleotides

Nucleotides are provided to enable the polymerase to extend the 3′ end of the stem-portion to produce the desired oligonucleotide, using the template to provide the sequence. The nucleotides may comprise adenine, cytosine, thymine, guanine, and uracil. The nucleotides may be natural (non-modified) nucleotides or may be modified or non-natural nucleotides, or nucleotide analogs, or a combination thereof. Non-naturally occurring analogs (or modified nucleotides) may include nucleotides comprising modified bases, sugars, or internucleosidic linkages, for example phosphorothioate internucleosidic linkages, 5′-N-phosphoramidite linkages, bases containing linking groups permitting the attachment of labels, such as fluorophores, or haptens, and the like. Whenever the use of an oligonucleotide or polynucleotide requires enzymatic processing, such as extension by a polymerase, ligation by a ligase, or the like, one of ordinary skill would understand that oligonucleotides or polynucleotides in those instances would not contain certain analogs of internucleosidic linkages, sugar moieties, or bases at any or some positions.

In some embodiments, a nucleic acid analog is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, 2′-deoxynucleoside 5′-triphosphates (dNTPs) analogues including Sp-dNTPaS, Rp-dNTPaS, Sp-2′F-dNTP, and dNTP-aS, methylated bases, intercalated bases, and combinations thereof. In some embodiments, a nucleic acid analog comprises one or more modified sugars (e.g., 2′-fluororibose, 2′-methoxyribose, 2′-methoxyethoxyribose, ribose, 2′-deoxyribose, arabinose, hexose or Locked Nucleic acids) as compared with those in commonly occurring natural nucleic acids.

Suitably, a plurality (or population) of nucleotides is provided. The plurality of nucleotides may or may not be bases of the same type (e.g., A, T, G, C, U etc.). For example, the solution may or may not comprise bases of only one type. The solution may comprise at least 1 type of base or bases of at least 2, at least 3, at least 4, at least 5, at least 6, at least 7, at least 8, at least 9, or at least 10 types. For instance, the solution may comprise any possible mixture of A, T, C, U and G. Any possible mixture of A, T, C, and G may be provided. A plurality of natural nucleotides and non-natural nucleotides may or may not be bases of the same type (e.g., A, T. G, C). In some instances, the solution may comprise a plurality of natural nucleotides and non-natural nucleotides. The plurality of natural nucleotides and non-natural nucleotides may or may not be bases of the same type (e.g., A, T, G, C). A plurality of nucleotides may be substantially all natural nucleotides, or may be substantially all modified nucleotides, either as described herein or known to the skilled person. A plurality of nucleotides may be a mixture of natural and modified nucleotides. In such a mixture, the ratio of modified: natural nucleotides may depend upon the nature of the oligonucleotide to be produced, and the proportion of modified nucleotides therein. A suitable ratio of modified: natural nucleotides may be 1:99, 5:95; 10:90; 20:80; 30:70: 40:60; 50:50. 60:40: 70:30; 80:20; 90:10; 95:5 or 99:1, or any range between any of the aforementioned upper and lower ranges, or any integer inbetween.

One or more nucleotides of the plurality of nucleotides may be labelled with a dye, fluorophore, or quantum dot. For example, the solution may comprise labelled nucleotides. In another example, the solution may comprise unlabelled nucleotides. In another example, the solution may comprise a mixture of labelled and unlabelled nucleotides.

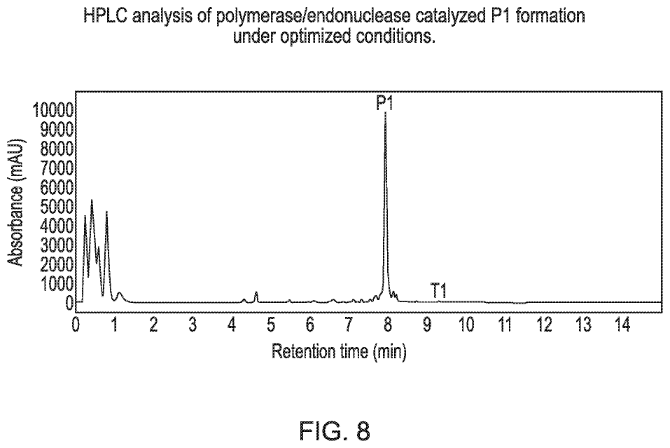

The nucleotides may be provided in any suitable concentration or amount, which may be calculated based on the length of the template and the expected number of rounds of oligonucleotide synthesis in a single reaction. The nucleotides may be provided in a suitable amount such that at least 30%, 40%, 50%, 60%, 70%, 80% or 90% or substantially all of the nucleotides provided are used in the reaction. Therefore the amount of nucleotides may be calculated for each reaction, based on the knowledge of the skilled person. Alternatively the nucleotides may be provided in excess, for example where the number of rounds of extension has not been pre-determined. The nucleotides may be provided in a 1.5 fold, 2 fold, 3 fold, 4 fold or fivefold excess, compared to the calculated amount of nucleotides needed for the desired number of oligonucleotides to be produced in the reaction. By way of example, the ‘optimized’ reaction as shown in FIG. 8 uses 4 uM template and completed 53 cycles to produce 212 uM product. The oligonucleotide product contains 18 nucleotides, so the reaction used 3.78 mM dNTPs. The reaction conditions provided 16 mM nucleotides, i.e. a 4-fold excess.

### III) Polymerase

A polymerase for use in the invention may be any suitable enzyme which is capable of synthesising a chain of nucleotides. A polymerase may be an RNA polymerase or a DNA polymerase. Suitably, a polymerase for use in the present invention may be one which is capable of incorporating modified or non-natural nucleotides into a nucleic acid chain. A polymerase may be naturally occurring or may be engineered. A suitable polymerase may be eukaryotic or prokaryotic.

A suitable polymerase may be one which is able to tolerate high concentrations on dNTPs without substantially affecting its activity or performance. Examples of such polymerases are described herein. An example of a polymerase which is able to tolerate high concentrations of dNTP's is KOD or variants thereof.

A suitable polymerase may be a thermostable polymerase, for example Taq polymerase, pfu from Pyrococcus furiosus or KOD from Thermococcus kodakaraensis (Tkod-pol), or variants or engineered versions thereof. Other suitable polymerases which may be suitable for use in the present invention include the Klenow fragment from E.coli, T4 and T7 polymerase, SFP1, Stoffel variant SF4-6, Stoffel homologs from Thermus filiformis and Marinithermus hydrothermalis (Mhpol), and the family B polymerase 9° N, Q5 DNA polymerase, and variants or engineered versions thereof. A list of engineered polymerases which may be suitable for use in the present invention is provided in Trends in Biotechnology, October 2019, Vol. 37, No. 10 https://doi.org/10.1016/j.tibtech.2019.03.011 or will be known to persons skilled in the art. A variant may have one or more improved features suitable for the present invention, for example improved fidelity, stability, or ability to provide substantially single stereoisomers. Suitable variants include for example SF4-6, TfPol* and MhPol* as provided herein. Most suitably, a SF4-6, TfPol* and MhPol* may be useful in generating a population of oligonucleotides of substantially the same stereoisomer.

Also provided herein is a polymerase having a sequence as defined in Table 3, or a sequence having at least 80%, 85%, 90% or 95% or more sequence identity thereto. A suitable modified polymerase will be capable of extending an RNA or DNA sequence; will be capable of accepting one or more modified nucleotides into a nucleotide chain; and will have one or more of the nucleotide mutations as described in Table 3. Such a polymerase may be used in a method of the invention, or may be provided in a kit of the invention. A polymerase of the invention may be one which is able to withstand high concentrations of nucleotides, such that concentrations of nucleotides above 3, 5, 8, 10, 15, 20, 25, 30, 35, 40, 45 or 50 mM do not substantially inhibit or affect the polymerase activity.

Any suitable amount of polymerase may be used in the reaction. The amount of polymerase may be selected based upon features 0.1-5 of the reaction such as the amount of product to be generated, the concentration of nucleotides, the nature and efficiency of the polymerase, and the nature of the cleaving agent. A skilled person will be capable of determining a suitable amount of primer using the information provided herein, and his common general knowledge. Suitably, no more than 10 mol %, 9 mol %, 8 mol %, 7 mol %, 6 mol %, 5 mol %, 4 mol %, 3 mol %, 2 mol % or 1 mol % polymerase may be used in a method of the invention. A suitable range may be 0.1-5 μM.

A polymerase for use in the present invention may be produced by any suitable method, for example a polymerase may be produced recombinantly in a host cell. A suitable host cell will be known in the art, and may be a microbial cell, a mammalian cell, or a cell line. A suitable host cell may be a bacterial cell, for example, E. coli.

### V Cleaving Agent

A reagent of the present invention includes a suitable cleaving agent which can mediate cutting of the extended nucleic acid molecule at a cleavable site, to enable dissociation of the synthesised oligonucleotide from the nucleic acid molecule template. A cleaving agent is suitably site specific, so that it cuts the extended primer-template at a particular site.

References herein to ‘cleaving agent” refers to a substance which is able to cut the bond between the newly formed oligonucleotide and the primer. A suitable cleaving agent for use in the present invention suitably will cut the oligonucleotide from the primer-template and leave a free 3′ OH group at the end, such that the primer-template can be recycled and used again as a template for oligonucleotide synthesis. A cleaving agent may be a chemical cleaving agent, an enzymatic cleaving agent, or a photo cleaving agent, or any other suitable reagent or system. Suitably, the cleaving agent is an enzymatic cleaving agent.

A suitable cleaving agent may be one which is able to tolerate high concentrations on dNTPs without substantially affecting its activity or performance. Examples of such cleaving agents are described herein, for example an enzymatic cleaving agent, for example endonuclease V, suitably TnEndoV.

Examples of suitable enzymatic cleaving systems include any enzyme which can cut a single strand of nucleic acid, for example restriction enzymes or endonucleases. Examples of suitable endonucleases for use in the present invention include TmEndoV from Thermotoga maritima (TmEndoV), TmEndoV from Thermotoga neapolitana, PtEndov from Pseudothermotoga thermarum, TmeEndoV from Thermosipho melanesiensis, and TaEndoV from Thermosipho atlanticus, or nicking endonucleases Nt BstNBI, NtAlwl and Nt BspQI. The protein sequences can be found in Table 2. Also included are endonucleases being substantially identical to an endonuclease of Table 2.

An enzymatic cleaving agent such as an endonuclease may be one which is able to withstand high concentrations of nucleotides, such that concentrations of nucleotides above 3, 5, 8, 10, 15, 20, 25, 30, 35, 40, 45 or 50 mM do not substantially inhibit or affect the enzyme activity.

A preferred endonuclease is specific for deaminated bases such as inosine. Endonuclease V will cleave/hydrolyse the second phosphodiester bond 3′ to inosine.

The cleaving can be done in a single step, or can be a multi-step process.

Where the cleaving agent requires binding of an enzyme or reagent to the nucleic acid molecule, it is anticipated that the endonuclease will not bind to the template until the polymerase is released to avoid steric limitations due to the size of the polymerase.

For cleavage by an endonuclease, the product needs to be double stranded for binding and cleaving. In a further embodiment, the cleavage solution comprises one or more buffers. It will be understood by the person skilled in the art that the choice of buffer is dependent on the exact cleavage chemistry and cleaving agent required.

Cleavage may usually be carried out in an aqueous system. Alternatively, cleaving may take place in the presence of an alternative nucleophile, such that the alternate nucleophile is incorporated into the 5′ phosphate group of the oligonucleotide product. This nucleophile incorporation provides a modified oligonucleotides suitable for conjugation to delivery vehicles such as antibodies, peptides, proteins or sugars for targeted delivery to specific organs. Any suitable nucleophile may be used, for example and without limitation, glycerol, ethylene glycol, 1, 2, propandiol, or 1, 3-diaminopropanol. Suitable concentrations may be determined to achieve the desired incorporation. For example, at least 30%, 40%, 50%, 60%, 70%, or 80% w/v may be used, or any suitable range between the afore-mentioned integers.

### VI) Other Reagents

Additional reagents may be provided, including for example and without limitation, washing solution, buffers, primers, enzymes, catalysts, quenchers, dyes, probes, tags, labels, co-factors,, fluidic components (e.g., surfactant, buffer, triton X-100, nonidet P-40, DMSO etc.), and/or optical components (e.g., reference beads, dyes, etc.), and any one or more of magnesium ion (Mg²⁺), manganese ions (Mn²⁺), glutamine, arginine, tetramethyl ammonium chloride, betaine, formamide, bovine serum albumin or any combination thereof.

A widely used dNTP backbone variant is phosphorothioate (also referred to as S oligo), where the phosphodiester bond is converted to a phosphorothioate bond. The sulphurisation process results in R and S diastereomers. Applying different co-factors to the reaction mixture can be used to direct synthesis to the R or S stereoisomer. For example, where a polymerase naturally accepts the S isomer, it may be suitable to apply a co-factor to direct the polymerase toward the R isomer, thereby resulting in the use of both forms. For example, where KOD is used, it may be suitable to use in combination a cobalt co-factor. A suitable reagent for use in the reaction may be a cobalt salt for example cobalt chloride. Other suitable combinations of polymerases and co-factors may be determined using the teaching herein and available in the field.

A suitable buffer for use in the invention is one which provides aqueous conditions. Therefore the buffer is suitably an aqueous buffer. A suitable buffer may comprise one or more reagents selected from Tris-HCL, Hepes, ammonium sulphate, potassium chloride, calcium chloride, magnesium sulphate, glutamine, arginine, formamide, nonidet and Triton X-100. A suitable buffer may comprise Tris-HCL, (NH₄)₂SO₄, KCI, Triton X-100, and MgSO₄,. For example, a suitable buffer may comprise 20 mM Tris-HCI, 10 mM (NH₄)₂SO₄, 10 mM KCI, 2 mM MgSO₄, 0.1% Triton X-100, pH 8.8. Another suitable buffer may comprise Tris-HCL, MgCI₂, potassium glutamate, arginine-HCI, acetylated BSA, DTT, trehalose and 1,2-propanediol. For example, a suitable buffer may comprise 20 mM Tris-HCI (pH 8), 20 mM MgCI₂, 100 mM potassium glutamate, 100 mM arginine-HCI, 0.01 mg/ml acetylated BSA, 10 mM DTT, 0.2 M trehalose and 1 M 1,2-propanediol. Another suitable buffer may comprise Tris-AcOH, MgSO₄ KOAc, potassium glutamate, arginine-HCI, acetylated BSA, Triton X-100 and DTT. For example, a suitable buffer may comprise 50 mM Tris-AcOH (pH 8), 20 mM MgSO₄, 50 mM KOAc, 100 mM potassium glutamate, 100 mM arginine-HCI, 0.01 mg/ml acetylated BSA, 0.1% Triton X-100 and 30 mM DTT. Another suitable buffer may comprise Tris-HCI (pH 8.8), (NH₄)₂SO₄, KCI, MgSO₄, Triton X-100 and NiCI₂20. For example a suitable buffer may comprise 20 mM Tris-HCI (pH 8.8), 10 mM (NH₄)₂SO₄, 10 mM KCI, 2 mM MgSO₄, 0.1% Triton X-100 and 2 mM NiCI₂. Another suitable buffer may comprise Tris-HCI (pH 8), MgCI₂, potassium glutamate, arginine-HCI, acetylated BSA, DTT. For example, a suitable buffer may comprise 20 mM Tris-HCI (pH 8), 20 mM MgCI₂, 100 mM potassium glutamate, 100 mM arginine-HCI, 0.01 mg/ml acetylated BSA, 10 mM DTT. Alternative buffers may be identified by the skilled person using the teaching herein and available in the art.

Suitable combinations of buffers and enzymes may be determined using the teaching herein and available in the art. For example, any one of the above mentioned buffers may be used in combination with a polymerase selected from KOD or a variant thereof such as KOD DGLNK, or TfPol or a variant thereof.

Suitably, the reagents used in the method of the present invention are aqueous.

Suitably, the reagents used in the method of the present invention substantially do not comprise acetonitrile.

### Combinations

In the present invention, suitable reagents include a combination of enzymes (i.e. a polymerase and endonuclease), suitably in combination with any reagents, such as one or more reagents as defined herein which suitably are optimum for the generation of the desired oligonucleotide. Suitably, a reaction may comprise a primer-template, a polymerase, an endonuclease, dNTPs and a buffer. Suitably, a reaction may comprise a self-priming template, a polymerase, an endonuclease, dNTPs and a buffer. The polymerase may be KOD, TfPol*, KOD DGLNK, or SFM4-6. The endonuclease may be endonuclease V, suitably TnEndoV.

In an embodiment, the invention may comprise a polymerase selected from the group of Table 3, and an endonuclease selected from the group of Table 2. All such combinations are included within the scope of the invention, in combination with a primer-template or specifically a self-priming template. Suitably, the endonuclease is TnEndoV. Suitably, the nucleotides may comprise one or more types of modified nucleotide. Suitably, the cleavable site is a cleavable nucleotide, for example inosine.

In an embodiment, a polymerase may be a modified polymerase as shown in Table 3, and the endonuclease may be endonuclease V. Suitably, the endonuclease is ThEndoV. One or more nucleotides may be modified nucleotides. Suitably, the cleavable site may be an inosine residue. Suitably, the buffer may be as provided herein.

### Method

The present invention provides a method for producing a single stranded oligonucleotide, the method comprising:
i) providing a primer-template comprising a) a primer for initiation of oligonucleotide synthesis, b) a template which directs synthesis of an oligonucleotide product, and c) a cleavable site to enable release of the oligonucleotide product from the template;
ii) incubating the primer-template with a nucleic acid polymerase, one or more dNTPs, and a cleaving agent to form a reaction mixture;
iii) maintaining the reaction mixture under conditions to allow for extension of the primer by the polymerase to form an extended primer-template, and cleavage of the extended primer-template at the cleavable site by the cleaving agent to provide an oligonucleotide product;
iv) optionally separating the oligonucleotide product from the reaction mixture.

The primer-template, polymerase, dNTPs, and/or cleaving agent may each individually be as described herein. Any suitable combination of primer-template, polymerase, dNTPs, and/or cleaving agent as described herein may be used in the method of the invention.

The nucleotides may be natural nucleotides, or may comprise one or more modified nucleotides, for example as described herein. The nucleotides may be provided in excess of the amount required, for example as described herein.

In a suitable embodiment, the method is for making a therapeutic oligonucleotide. Suitably, the cleaving agent is TnEndoV and the nucleotides comprise one or more modified nucleotides.

The method of the invention may be a single-step method, or may comprise two or more sequential steps. In a single step method, all reagents may be combined in a single step, under conditions suitable for the extension and cleavage reactions to proceed. The method of the invention may be a one-pot method, wherein extension and cleavage takes place in a single reaction vessel.

Maintaining the reaction mixture as defined in step iii) may be for a reaction period, suitably a defined reaction period. During the defined reaction period, both extension and cleavage may take place in the reaction mixture, suitably in the same reaction period. Therefore, the reaction may comprise repeated cycles of extension and cleavage, within the same reaction mixture in a reaction period. In this manner, the primer-template is continually re-used.

Suitably, the extension and cleavage may take place under the same set of reaction conditions. The reaction conditions may comprise the presence of a suitable buffer and one or more additional reagents as defined herein, at a suitable temperature for a pre-defined reaction period.

Suitably, the extension and cleavage may take place Any preceding or subsequent steps to iii) above may be performed in a separate step and/or vessel, or in the same step/and/or vessel as steps i) to iv). The conditions, or any part of the conditions, for any preceding or subsequent steps may be the same or may be different.

The method of the invention may comprise two or more methods carried out in parallel, for example in an array or multiple arrays. Each reaction may comprise the same or a different template.

A method of the invention may be conducted under any suitable conditions to allow extension of the primer sequence to generate a new oligonucleotide sequence; and suitably retention of the primer-template duplex. Where a self-priming template is used, the conditions suitably allow for maintenance of the secondary structure for the duration of the reaction period, for example as a hairpin loop. Suitable conditions include a suitable temperature, pH, buffer, incubation time and salt concentration. The conditions for any step of the method may be the same as for one or more other steps, or may be different.

Step iii) may be performed at a temperature which is at or above the melting temperature of the template and cleaved product. At such a temperature, the extension product (oligonucleotide product) is released from the primer-template after cleavage, leaving the primer-template available for another extension cycle. The primer however, remains bound and does not dissociate during the reaction conditions. A suitable temperature may be calculated by a person skilled in the art based upon factors such as the length of the template and extension product, and the sequence thereof, and the optimum temperature for the polymerase and cleaving agent. Longer oligonucleotide products have a higher melting temperature than shorter products. A suitable temperature when using a self-priming template for synthesis of a longer oligonucleotide product, for example an 18 mer, may be 60-85° C., or any integer or range therebetween. A more suitable temperature range may be 65-80° C., or any integer therebetween, for example about 70° C. If the target therapeutic oligonucleotide was shorter a lower temperature could be used, for example 45 to 60° C., or any integer or range therebetween. Alternatively, the temperature may be cycled between a first temperature suitable for primer extension, and a second temperature for cleaving

The combination of providing the polymerase and cleaving agent in the same reaction, under conditions which allow for dissociation of the oligonucleotide product from the primer-template allows for the primer-template to be used repeatedly in the reaction. The primer-template may be referred to as being catalytic, because it mediates an increase in the product beyond the usual 1:1 ratio of template:product in a conventional extension reaction and because it remains unchanged by the extension or cleavage steps.

The method of the present invention therefore enables the generation of oligonucleotide product in amounts of over 1×, or at least 2×, 3×, 4×, 5×, 10×, 20×, 30×, 40×, 50×, 100× or 200×, 300×, 500× or 1000× product compared to the amount of primer-template at the start of the reaction. The amount may be expressed as a mol %.

The extension reaction may take place at a suitable pH, which represents a suitable pH for the activity of the polymerase, for example between 8.0 and 9.5. A suitable buffer such as Tris-HCL or having on or more buffer reagents as described herein, or an equivalent known to a person skilled in the art may be provided to stabilise the pH.

The time for the reaction will depend upon the amount of product required, and the number of extension cycles. A typical extension time may be 1 to 2 minutes per cycle. A reaction may be incubated for several minutes to several hours, or up to a day or more to achieve a suitable yield. An optimum incubation time for extension can be calculated for each reaction. An optimum reaction time may be 12 hours or more. A reaction time may be based upon the number of extension cycles, which may be 2 or more, 10 or more, 50 or more, 100 or more, 200 or more, 300 or more, 500 or more, or 1000 or more. The method may allow for accumulation of the oligonucleotide product in the reaction mixture.

The conditions under which the extension reaction is performed may be aqueous. In an embodiment, the conditions substantially do not comprise acetonitrile.

A method may comprise providing a suitable buffer in with which the reagents are combined for performance of the extension reaction. A suitable buffer may comprise one or more components selected from buffer salts (for example Tris-HCI, Tris-AcOH, HEPES-KOH, Bicine-HCI), metal ions (for example MgSO₄, MgCI₂, MgOAc₂, CaCI₂), amino acids (for example glutamate, arginine, proline, glycine, serine, aspartate) and additives (for example sodium citrate, (NH₄)₂SO₄, tetra-methylammonium chloride (TMAC), KCI, KOAc, glycerol, 1,2-propanol, sucrose, trehalose, acetylated BSA, Tween-20, Triton X-100, DTT, formamide, DMSO and/or PEG-3000). A suitable buffer may comprise magnesium, potassium chloride, Tris-HCL, ammonium sulphate, one or more co-solvents including for example DMSO, glycerol, formamide, BSA, PEG, gelatin, non-ionic detergent (TWEEN 20 or Triton-X-100) and N,N,N trimethylglycine. A most suitable buffer may comprise 20 mM Tris-HCI, 10 mM (NH₄)₂SO₄, 10 mM KCI, 2 mM MgSO₄, 0.1% Triton X-100, pH 8.8. Another suitable buffer may comprise 50 mM Tris AcOH, 20 mM MgSO₄, 50 mM KOAc, 100 mM KGlu, 100 mM ArgHCI, 0.01 mg/ml AcBSA, 0.1% Triton X-100, 30 mM DTT, 5% formamide. Other suitable buffers are described herein, or may be known and available to persons skilled in the art.

Where the polymerase is KOD, the cleaving agent may be endonuclease V, for example TnEndoV (2 μM) catalyzed amplification are 20 mM Tris-HCI (pH 8), 20 mM MgCI₂, 100 mM arginine-HCI (pH 8), 100 mM glutamate-KOH (pH 8), DTT (10 mM), formamide (10%), trehalose (0.2 M), 1,2-propanediol (1 M) and acetylated BSA (0.01 mg/ml). An example of optimised conditions for a larger scale reaction is a buffer containing 50 mM Tris AcOH, 20 mM MgSO4, 50 mM KOAc, 100 mM KGlu, 100 mM ArgHCI, 0.01 mg/ml AcBSA, 0.1% Triton X-100, 30 mM DTT, 5% formamide, pH 8.0, using Sₚ-dGTPaS (2.5 mM), Sₚ-dCTPaS (2.5 mM), Sₚ-dTTPaS (5 mM), template (4 μM), KOD (2 μM), TnEndoV (6 μM) and 0.012 U/μl TIPP (Inorganic pyrophosphatase); incubation at 70° C. for 12 hours or more; quenching by heating to 98° C. for 2 h.

The amount of enzyme and nucleotides may be empirically determine based on the nature of the polymerase, and concentrations of nucleotides, presence of chelating agents and proteins, for example. Any reagent may be provided in excess.

Cleaving may be carried out enzymatically or by light-based cleavage, or any other suitable mechanism. The conditions required for cleavage will depend upon the nature of the cleaving system used and will be known to a person skilled in the art. Most suitable conditions are those which do not affect, or substantially affect, the function of any reagents, such as enzymes, in the reaction, so that multiple cycles are possible.

A method of the invention may be used to produce double stranded DNA, for example by performing a method of the invention to produce two complementary strands of single stranded nucleic acids, and performing an annealing step to produce a double stranded oligonucleotide.

A method of the invention may also include one or more additional steps, for example, stopping, terminating or quenching the reaction, extracting proteins; extracting the product; washing the product, purification of the product, modifying or adapting the oligonucleotide product, for example by ligation to another molecule or cleavage into different oligonucleotides. The method may comprise preserving the product, for example by freeze drying, packaging and/or storing the product.

Suitable methods and conditions for purification will be known to the skilled person. Any suitable method may be used to extract the oligonucleotide product, suitably without the primer/template nucleic acid molecule. Examples of suitable methods are described herein and include selective crystallization, ethanol/chloroform precipitation or using an established filtration system.

### Solid Support/Array

The present invention provides for one or more primer-templates of the present invention to be provided on a support, for example a solid substrate. Immobilisation of the nucleic acid molecule on a surface may be reversible or non-reversible. Any suitable immobilisation method may be used, either by way of a nucleic acid sequence which binds to a surface, or by way of incorporation of a suitable moiety which mediates binding to the surface. For example, a suitable binding motif may include a sequence which binds to a complementary sequence on the solid support, such as a polyA sequence.

Binding may be mediated via an immobilisation site in the nucleic acid molecule and a complementary site or binding moiety provided on the solid support. Alternatively, the nucleic acid sequence may be attached to the support non-covalently, for example by physiosorption.

The substrate may be a solid substrate. The substrate may entirely or partially comprise one or more of rubber, glass, silicon, a metal such as aluminum, copper, titanium, chromium, or steel, a ceramic such as titanium oxide or silicon nitride, a plastic such as polyethylene (PE), low-density polyethylene (LDPE), high-density polyethylene (HDPE), polypropylene (PP), polystyrene (PS), high impact polystyrene (HIPS), polyvinyl chloride (PVC), polyvinylidene chloride (PVDC), acrylonitrile butadiene styrene (ABS), polyacetylene, polyamides, polycarbonates, polyesters, polyurethanes, polyepoxide, polymethyl methacrylate (PMMA), polytetrafluoroethylene (PTFE), phenol formaldehyde (PF), melamine formaldehyde (MF), urea-formaldehyde (UF), polyetheretherketone (PEEK), polyetherimide (PEI), polyimides, polylactic acid (PLA), furans, silicones, polysulfones, any mixture of any of the preceding materials, or any other appropriate material. The substrate may be entirely or partially coated with one or more layers of a metal such as aluminum, copper, silver, or gold, an oxide such as a silicon oxide (SixOy, where x, y may take on any possible values), a photoresist such as SU8, a surface coating such as an aminosilane or hydrogel, polyacrylic acid, polyacrylamide dextran, polyethylene glycol (PEG), or any combination of any of the preceding materials, or any other appropriate coating.

A surface of the substrate may be modified to comprise any of the immobilisation reagents or binding partners described herein. A surface of the substrate may be modified to comprise active chemical groups, such as amines, esters, hydroxyls, epoxides, and the like, or a combination thereof. In some instances, such binding partners, chemical agents, proteins, nucleic acid sequences or surface modifications may be added as an additional layer or coating to the support.

The substrate may have the general form of a cylinder, a cylindrical shell or disk, a rectangular prism, or any other geometric form. A surface of the substrate may be planar. A surface of the substrate may be uncovered and may be exposed to an atmosphere. Alternatively or in addition, a surface of the substrate may be textured or patterned. For example, the substrate may comprise grooves, troughs, hills, and/or pillars. The substrate may define one or more cavities (e.g., micro-scale cavities or nano-scale cavities). The substrate may define one or more channels. The substrate may have a regular textures and/or patterns across the surface of the substrate. For example, the substrate may have regular geometric structures (e.g., wedges, cuboids, cylinders, spheroids, hemispheres, etc.) above or below a reference level of the surface. Alternatively, the substrate may have irregular textures and/or patterns across the surface of the substrate. For example, the substrate may have any arbitrary structure above or below a reference level of the substrate.

The substrate may comprise an array. For instance, the array may be located on a lateral surface of the substrate. The array may be a planar array. The array may have the general shape of a circle, annulus, rectangle, or any other shape. The array may comprise linear and/or non-linear rows. The array may be evenly spaced or distributed. The array may be arbitrarily spaced or distributed. The array may have regular spacing. The array may have irregular spacing. The array may be a textured array. The array may be a patterned array. The array may comprise a plurality of individually addressable locations.

The primer-templates may be immobilized to the array. The array may therefore comprise one or more binding partners, or means to mediate immobilisation of the primer-templates, as described herein. Such means may comprise one or more physical or chemical linkers or adaptors. Alternatively or in addition, the primer-templates may be coupled to a bead, which bead may be immobilized to the array.

Any number of primer-templates may be immobilized to the support. For example, the support may have immobilized thereto at least 1, at least 2, at least 3, at least 4, at least 5, at least 6, at least 7, at least 8, at least 9, at least 10 samples, many thousands, tens of thousands, hundreds of thousands, millions, hundreds of million, thousands of millions or more primer-templates may be immobilized.

The primer-templates may be immobilised through non-specific interactions, such as one or more of hydrophilic interactions, hydrophobic interactions, electrostatic interactions, physical interactions (for instance, adhesion), and the like. The nucleic acid molecule may be immobilised through specific interactions, for example via a binding partner. For instance, a binding partner may comprise oligonucleotide adaptors configured to bind to the nucleic acid molecule; or one or more of antibodies, oligonucleotides, nucleic acid molecules, aptamers, affinity binding proteins, lipids, carbohydrates, and the like. A suitable example is streptavidin, which may bind to a biotinylated nucleic acid molecule. A binding partner may immobilize biological analytes through any possible combination of interactions, for example through a combination of physical and chemical interactions, through a combination of protein and nucleic acid interactions, etc.

A support may comprise one or more features, and many binding partners can be present in the same feature on the support, for example many thousands, tens of thousands, hundreds of thousands, millions, hundreds of million, thousands of millions or more. The array may have a number of binders that is within a range defined by any two of the preceding values. In some instances, a single binding partner may bind a single primer-template. In some instances, a single binding partner may bind a plurality of primer-templates. In some instances, a plurality of binding partners may bind a single primer-template.

### Kit

An aspect of the present invention provides a kit. A kit may allow for the storage, transport, or delivery of one or more of the reaction reagents as described herein, suitably in appropriate containers, together with any additional components such as a substrate, support, and/or instructions for performing the method and/or supporting materials from one location to another. For example, a kit may include one or more enclosures (e.g., boxes) containing the relevant reaction reagents and/or supporting materials. Such contents may be delivered to the intended recipient together or separately. For example, a first container may contain a nucleic acid molecule for use in the present invention, while a second or more containers may contain nucleotides, a polymerase, and/or a buffer as described herein.

A kit may further include a support, suitably with a primer-templates as described herein attached thereto. The support may comprise a population of nucleic acid molecules attached thereto, for example as described herein. A support as provided in a kit may be a support as described above. A kit may comprise one or more supports as described herein. In each support or in each kit, the nucleic acid molecule may comprise a different template, for synthesis of a different oligonucleotide product.

### The Oligonucleotide Product

The method of the present invention is directed in the first instance to the generation of a single stranded oligonucleotide, which is cleaved and dissociated from the template sequence. By means of downstream processing, the single stranded oligonucleotide may be used to generate a double stranded oligonucleotide product.

An oligonucleotide generated by a method of the first aspect will be of substantially the same length as the template sequence. Therefore, the length of an oligonucleotide generated by steps i) to iv) of the method of the first aspect will be from 2 to 500 nucleotides in length, more suitable from 2 to 200 nucleotides in length. An oligonucleotide can be, for example 5 to 20, 21 to 30, 31 to 40, 41 to 50, 51-60, 61 to 70, 71 to 80, 80 to 100, 100 to 150 or 150 to 250 nucleotides in length.

The length of the oligonucleotide may be increased or decreased as a result of downstream processing, such as cleavage, splicing, or recombination.

A product of a method of the first aspect of the invention comprising the oligonucleotide may suitably be at least 80%, 85%, 90%, 91%, 92%, 93%, 94%, 95%, 96%, 97%, 98%, or 99% pure. Therefore, a product produced by a method of the first aspect of the invention may suitably have less than 20, 15, 10 or 5% contaminants. Contaminants may include products containing oligonucleotides with base substitutions, truncated sequences, and/or overextended sequences (containing additional bases), or products containing salts.

Reference to an oligonucleotide herein includes one or more oligonucleotides. Therefore, the method of the invention may produce one or more desired oligonucleotides, but multiple cycles of extension and cleaving. Therefore, a product of the method of the invention may be a population of oligonucleotides. Suitably, the population comprises single stranded oligonucleotides.

One of the most abundant modifications in a nucleic acid sequence is the introduction of a phosphorothioate (PS) linkage, where one of the non-bridging oxygen atoms within the phosphate groups is replaced by sulfur. This PS modification renders an oligonucleotide more stable to nucleolytic degradation and confers a substantial pharmacokinetic benefit through increased protein binding. Replacement of a phosphate with a phosphorothioate creates a chiral center at the phosphorus atom (Sp or Rp). Hence, the introduction of every PS linkage into an oligonucleotide results in the creation of two sets of diastereoisomers and, in the absence of a stereocontrolled synthesis, an N-mer phosphorothioate oligonucleotide is obtained as an inseparable mixture of 2N⁻¹ diastereoisomers, all of which have different and potentially opposing physico- and biochemical properties. The two stereoisomers are denoted S and R. When supplied with an Sp/Rp mixture of nucleotides, a polymerase as defined herein may use only the Sp-dNTPs to provide a product as a single diastereoisomer.

A population of oligonucleotides may be a population of mixed diastereoisomers (S and R), or may be substantially a single stereoisomer. A population may be substantially an S stereoisomer or an R stereoisomer. By substantially a single isomer is meant that the population comprises at least 80%, 85%, 90%, 95% or 99% a single stereoisomer.

An oligonucleotide synthesised by a method of the invention may be any oligonucleotide, for example one which has an industrial or therapeutic use, or has a use or role in research. In some embodiments an oligonucleotide synthesized by a method of the invention has enzymatic activity. In some embodiments an oligonucleotide synthesized by a method of the invention serves a mechanical function, for example in a ribonucleoprotein complex or a transfer RNA. In some embodiments an oligonucleotide synthesized by a method of the invention may function as an aptamer. In some embodiments an oligonucleotide synthesized by a method of the invention may be used for data storage. In some embodiments an oligonucleotide synthesized by a method of the invention may be a therapeutic oligonucleotide, for example an RNA which targets a nucleic acid (either RNA or DNA), an RNA which targets a protein, or an RNA which encodes a therapeutic protein. Therapeutic RNA oligonucleotides include: i) aptamers which are short single-stranded nucleic acids that can bind to variety of targets, such as proteins, peptides, carbohydrates, and other molecules, by virtue of the tertiary structure of the aptamer, rather than its sequence, for example Pegaptanib (Macugen, Bausch+Lomb Pharmaceutical Retina Portfolio), Emapticap pegol (NOXXON Pharma), olaptesed pegol (NOXXON Pharma), and REG1; ii) mRNA, for example as replacement therapy, where mRNA is administered to the patient to compensate for a defective gene/protein (for example without limitation AZD8601 (Moderna), mRNA-3704 (Moderna), MRT5005 (Translate Bio), mRNA-2416, mRNA-2752, and MEDI1191 (moderna), mRNA-2752, BNT131 (SAR441000), CV8102 (CureVac) or MEDI1191; or to supply therapeutic proteins; vaccination, where mRNA encoding specific antigen(s) is administered to elicit protective immunity (for example, without limitation, COVID-19 vaccines or cancer vaccines; or cell therapy, where mRNA is transfected into the cells ex vivo to alter cell phenotype or function, and then these cells are delivered into the patient (for example without limitation TriMix-based immunotherapy (ECI-006), MCY-M11 (MaxCyte); or iii) single stranded antisense oligonucleotide (ASOs) including either RNase H-dependent ASO or RNase H-independent (steric block) ASO, including for example and without limitation nusinersen (also known as spinraza; Ionis Pharmaceuticals), eteplirsen (Sarepta Therapeutics) and inotersen (Ionis Pharmaceuticals and Akcea Therapeutics); and RNAi (miRNA or siRNA), which are short single-stranded DNA, phosphorothioate DNA, RNA analogs, conformationally restricted nucleosides (locked nucleic acids, LNA), or oligonucleotides complementary to a certain region of RNA that they are meant to target, and which promote degradation or inhibit translation of proteins. microRNAs (miRNAs) are small non-coding RNA molecules that regulate the expression of multiple mRNAs by blocking translation or promoting degradation of the target mRNAs. siRNAs are small non-coding RNA duplexes that originate from precursor siRNAs. The method of the invention may be used to make double stranded oligonucleotides, for example where they may be synthesised separately and annealed under suitable conditions.

### Cells

A cell or a populations of cells may be provided which comprise a nucleic acid molecule of the invention, or a nucleic acid sequence which encodes a nucleic acid molecule of the invention. Suitable mammalian and bacterial cells are known to persons skilled in the art.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

The features of any one aspect described herein apply to the other aspects, mutatis mutandis.

### Examples

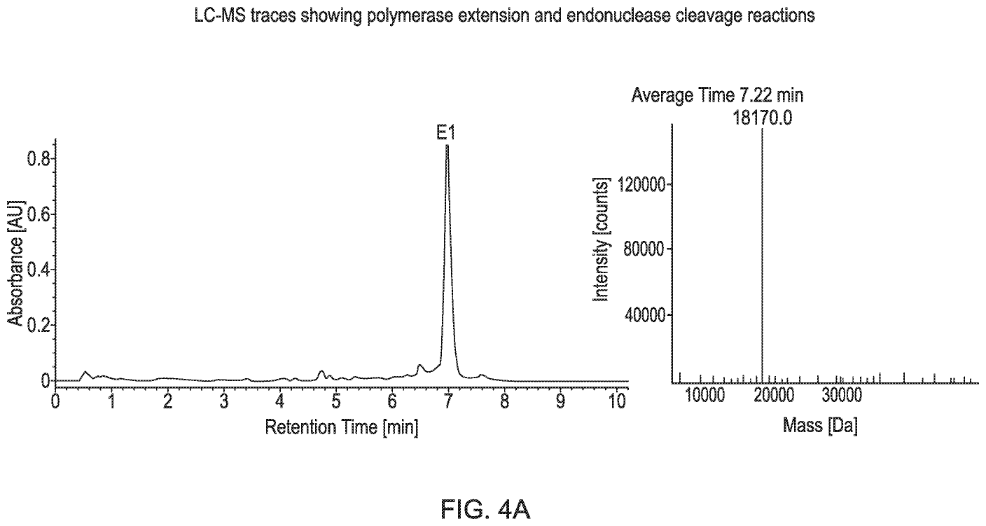

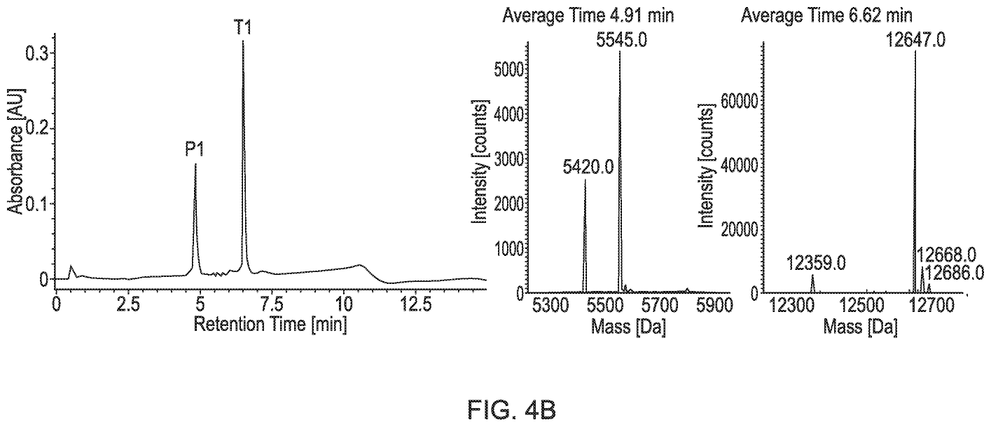

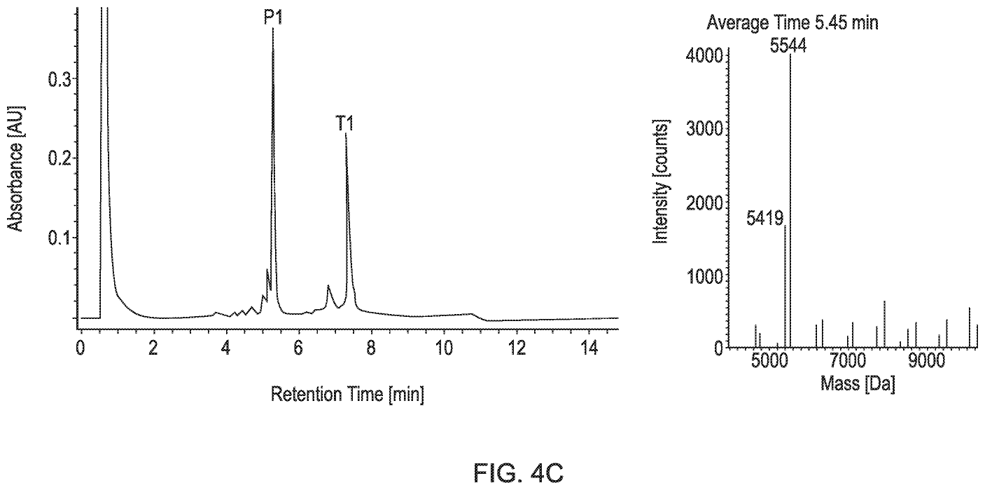

The extension and cleavage reactions were first evaluated in isolation using commercially available enzymes and the products were characterized by LC-MS analysis. The polymerase from Thermococcus kodakaraensis (KOD) successfully catalyzed the extension of the hairpin template (T1 of Table 1a, 20 μM) with unmodified dNTPs (1 mM) to afford the double stranded extended product (E1, Table 1a) in quantitative conversion (FIG. 4A). The cleavage reaction performed using a chemically synthesised standard of E1 (Table 1a, 20 μM) and an EndoV from Thermotoga maritima (TmEndoV) went to completion providing the 18 mer product (P1, Table 1) and template (T1, Table 1a) (FIG. 4B). Next, a one-pot polymerase-endonuclease reaction using KOD, TmEndoV, T1 (Table 1a, 20 μM) and dNTPs (1 mM) was performed which afforded 29 μM P1, representing 1.4 cycles of template extension and cleavage (FIG. 4C).

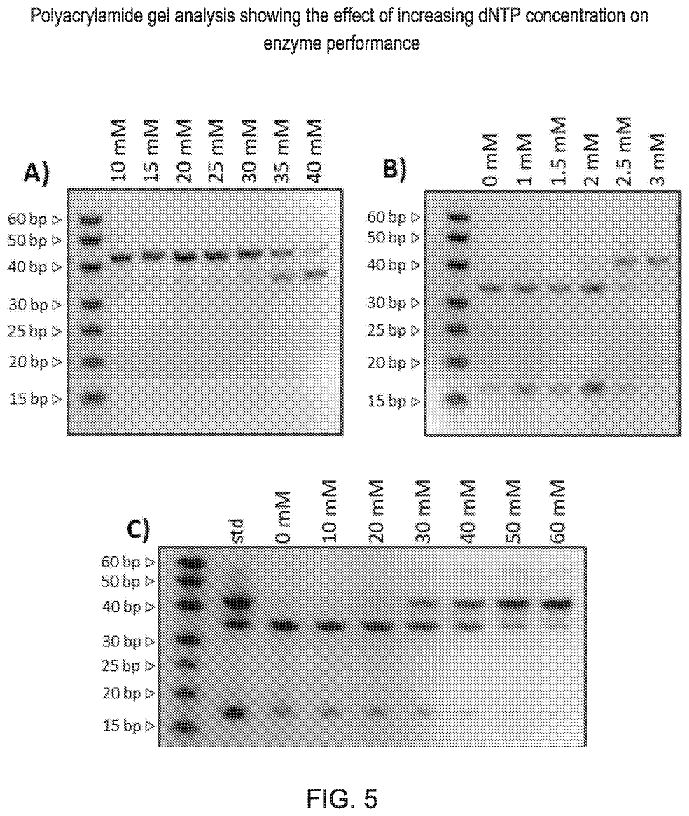

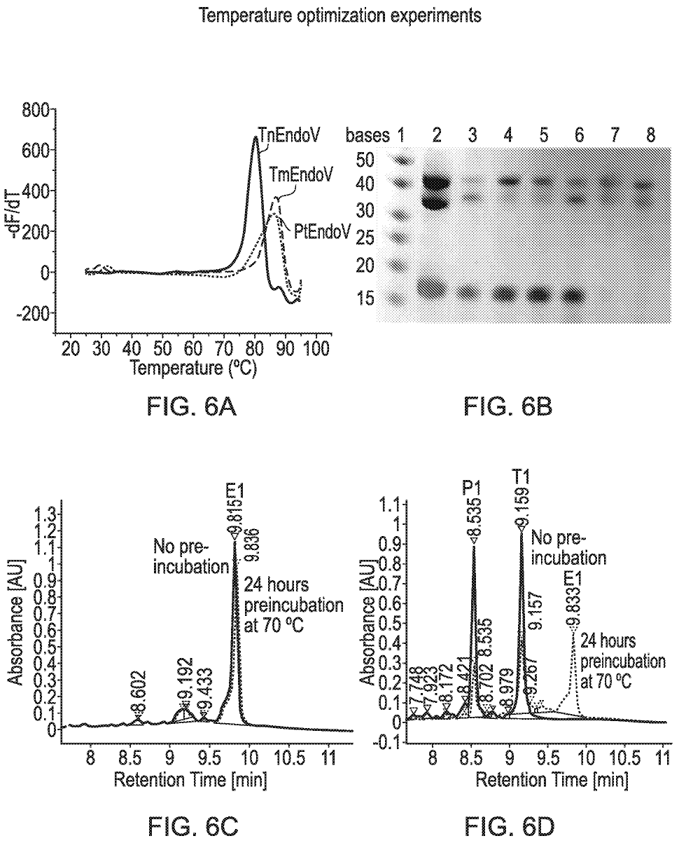

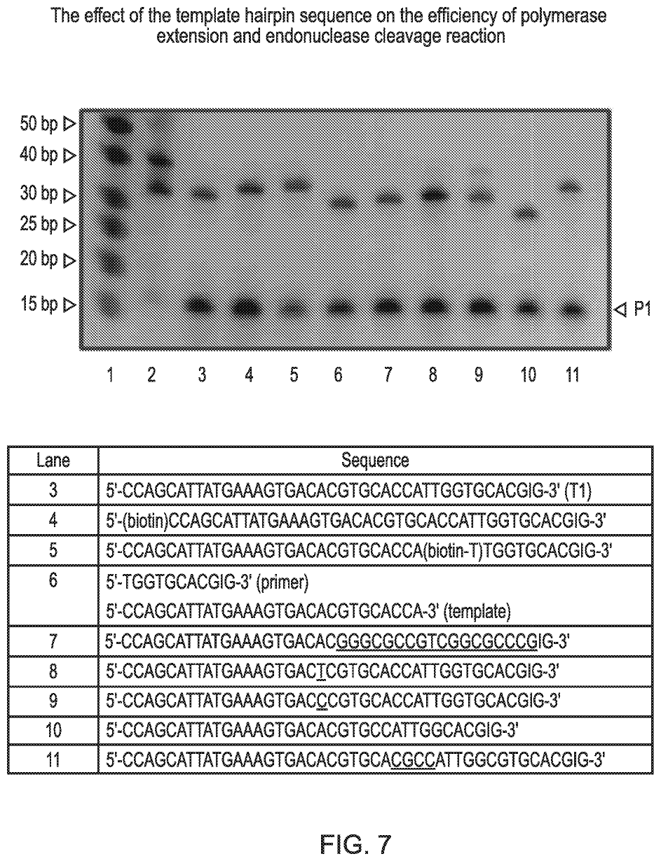

The effect of increased substrate (dNTP) loading on the polymerase extension and endonuclease cleavage reactions was investigated. KOD was able to efficiently extend T1 in the presence of 30 mM dNTPs (FIG. 5).) A panel of TmEndoV homologs (Table 2) identified from a BLAST search of the NCBI database was screened for improved activity in the presence of high dNTP concentrations. TnEndoV from Thermotoga neapolitana performed best at high dNTP concentrations and successfully hydrolyzed E1 (Table 1, 20 μM) in the presence of 30 mM dNTPs (FIG. 5B). To enable the cleaved product to effectively dissociate from the template to enable multiple cycles of template extension and cleavage, the process preferably operates above the product melting temperature. KOD and TnEndoV have optimal temperatures of >100° C. and 80.5° C. respectively (FIG. 6A) and retained 100% and 62% of their extension and cleavage activity following 24 hours pre-incubation at the optimal reaction temperature 70° C. (FIGS. 6C-6D). A panel of templates (T1-9) with different hairpin sequences were also evaluated in one-pot polymerase-endonuclease reactions (FIG. 7). The yield of product P1 (Table 1a) was largely unaffected by changes to the nucleobase pairing to inosine (T1, T6 & T7, Table 1a), the hairpin sequence (T5, Table 1a) or 5′-biotinylation (T2, Table 1a). However placing a biotin at the hairpin (T3) or varying the length of the hairpin (T8 & T9) led to a slight reduction in product yield. Lastly the effect of buffer composition on the efficiency of the one-pot reaction was investigated, 20 mM Tris pH 8, 50 mM KCI, 12 mM MgSO₄, 10 mM DTT, 0.1% BSA, 100 mM Arginine, 100 mM glutamate was selected as an optimal reaction buffer. A one-pot reaction was performed using the optimized process conditions and P1 formation was monitored by HPLC. After 18 hours, the system had completed 330 cycles of template (1 μM, 0.3 mol %) extension and product cleavage to afford 0.33 mM P1 (equivalent to 1.9 g/L) (FIG. 8).

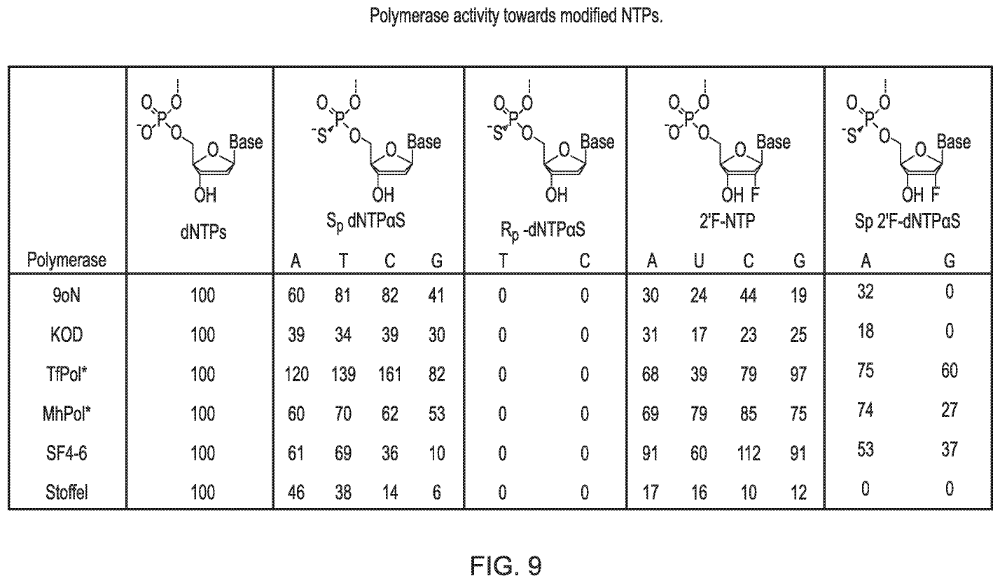

To further exploit this approach in the manufacture of therapeutic oligonucleotides, polymerases with activity towards nucleotide triphosphate building blocks containing pharmaceutically relevant modifications are required. A panel of polymerases was selected for evaluation including Stoffel, KOD and 9oN. A Stoffel variant (SF4-6) previously engineered to partially amplify 2′-methoxy and 2′-fluoro modified oligonucleotides was also included (Chen et al. Nat. Chem. 2016, 8, 556) A BLAST search of the NCBI database identified Stoffel homologs from Thermus filiformis (TfPol) and Marinithermus hydrothermalis (MhPol), and point mutations that were shown to enhance the substrate promiscuity of SF4-6 were introduced into TfPol and MfPol to afford TfPol* and MhPol* respectively. Polymerase activity towards modified nucleotide triphosphates was evaluated using a time-resolved FRET based assay which exploits a template modified with a FRET donor (fluorescein) and FRET acceptor (rhodamine). Reactions were performed using three natural dNTPs and one modified NTP. Enzymes able to transcribe the template and incorporate four copies of a modified NTP disrupt the template secondary structure leading to spatial separation of the donor and acceptor, resulting in a fluorescent signal. Control reactions performed in the absence of the modified nucleotide did not produce fluorescence demonstrating the high level of enzyme fidelity. All polymerases accepted 2′-fluoro modified NTPs (FIG. 9). 2′-MeO-NTPs are known substrates for SF4-6 (Chen et al. Nat. Chem. 2016, 8, 556) and LNA-NTPs are known to be accepted by KOD and 9oN (Veedu et al. Mol. BioSyst., 2009, 5, 787) the activity compared to natural dNTPs is below the detection limit of this assay.

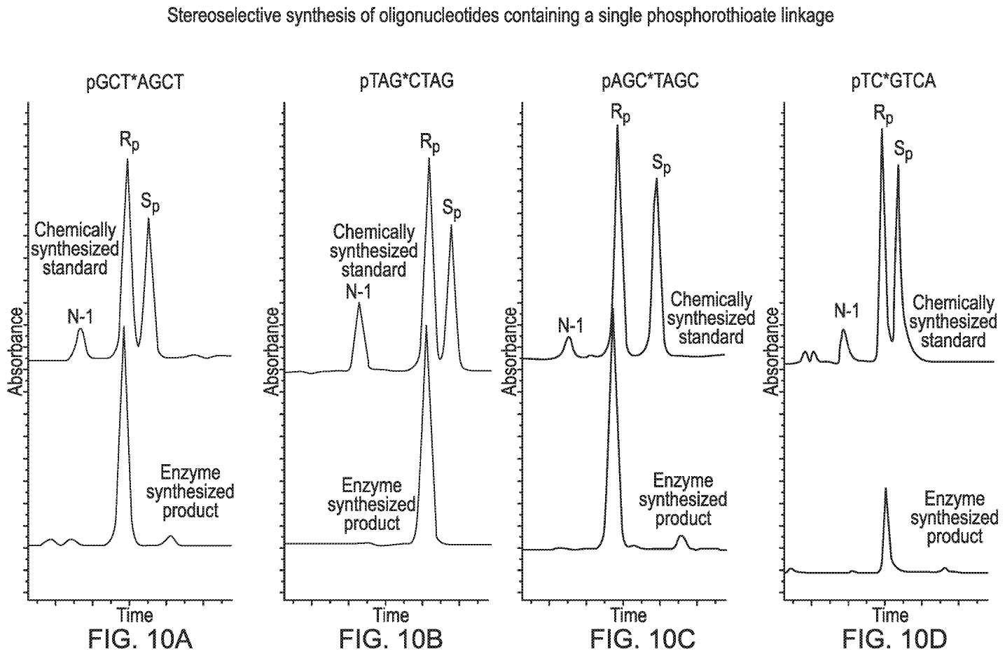

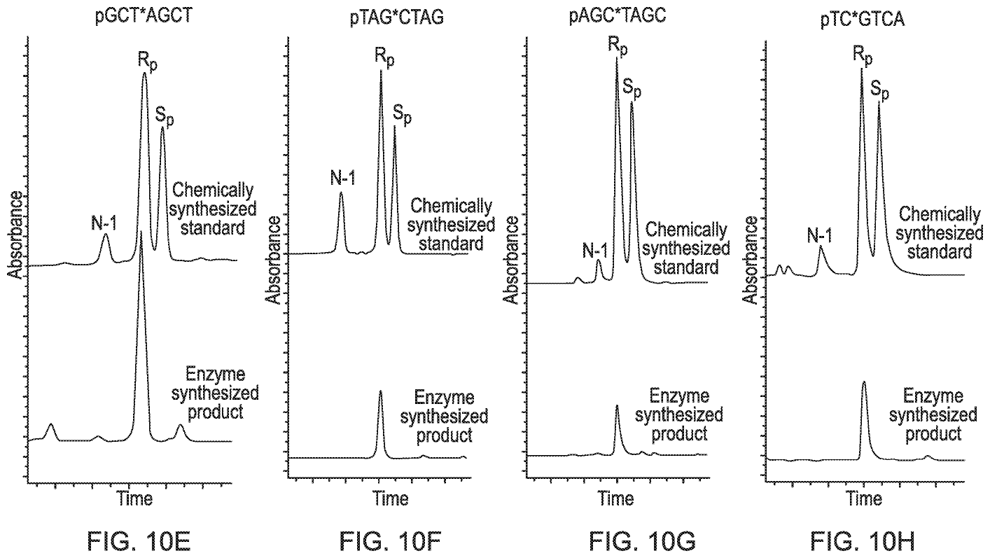

The panel of polymerases were screened for activity towards the single diatereoisomers of nucleotide thiophosphates (dNTPaS). All polymerases screened demonstrated activity towards the Sₚ diastereoisomers (Sₚ-dNTPaS), however neither Rp-dCTPaS or Rp-dTTPaS were accepted (FIG. 9). Variants SF4-6, TfPol* and MhPol* were also active towards the Sp diastereoisomers of 2′-F-ATP and 2′-F-GTP. To demonstrate the potential of our approach for the synthesis of stereopure therapeutic oligonucleotides, we performed one-pot polymerase endonuclease reactions using 3 natural dNTPs and an Sp-dNTPaS to generate products containing a single phosphorothiate linkage (P2-P5). Comparison of the enzymatically synthesized products (P2-P5) to diastereomeric mixtures produced by chemical synthesis showed that the biocatalytic process affords products as single diastereomers in >99% d.e. (FIGS. 10a-d). Polymerases are known to proceed with inversion of stereochemistry and so products generated from Sₚ-dNTPs will contain Rₚ linkages. The inventors were able to exploit polymerase stereospecificity to perform kinetic resolutions and produce oligonucleotide products as single stereoisomers from diastereomeric mixtures of dCTPaS or dATPaS (FIGS. 10e-h).

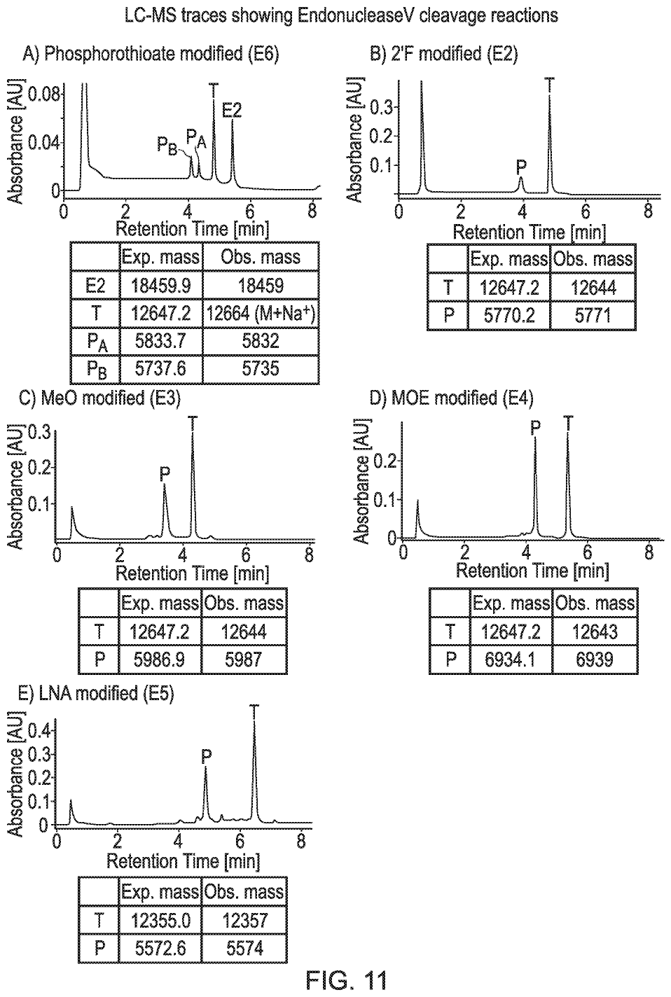

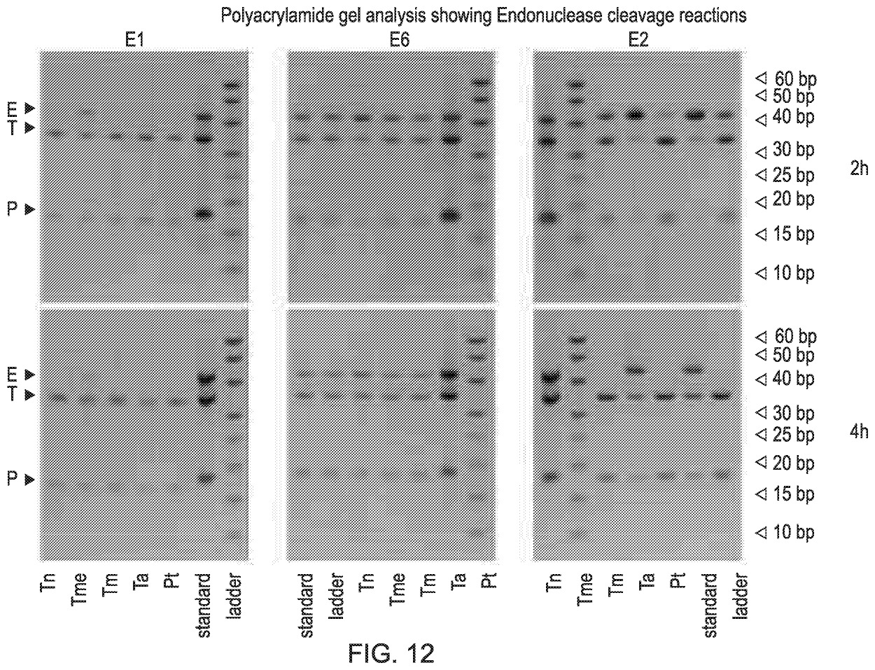

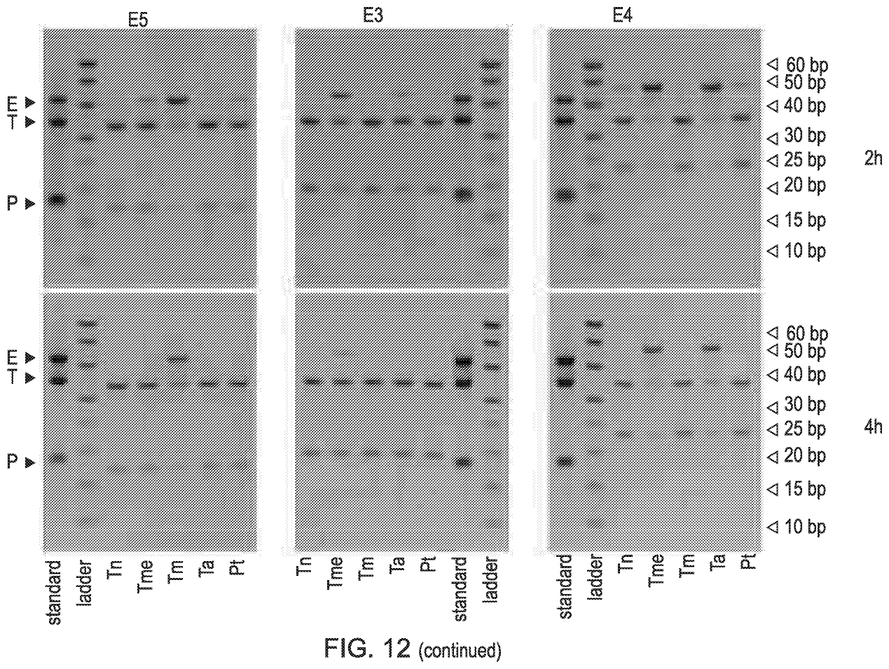

Next the panel of EndoVs were screened for activity towards chemically synthesized, extended templates (20 μM) containing 2′-fluoro (E2, Table 1a), 2′-methoxy (E3, Table 1a) and 2′-methoxyethoxy (E4, Table 1) ribose modifications, locked nucleic acids (E5, Table 1a) and phosphorothioate linkages (E6, Table 1). All EndoVs evaluated catalysed the cleavage of DNA containing 2′-ribose modifications 3′ of the cleavage site to provide the expected products P6-P10 (Table 1a) which were confirmed by LC-MS or PAGE analysis (FIGS. 11 & 12). Although TmEndoV, TnEndoV and PtEndoV afford P1, P6-P9 in >99% conversion within 18 hours, reactions catalysed by TnEndoV were fastest with almost complete conversion (>96%) achieved within 1 hour (FIG. 12). The LNA modified extended template (E5) contains only a single LNA 3′ of the cleavage site, as long LNA sequences are known to distort secondary structure, as such therapeutic oligonucleotides rarely contain more than three sequential LNAs.

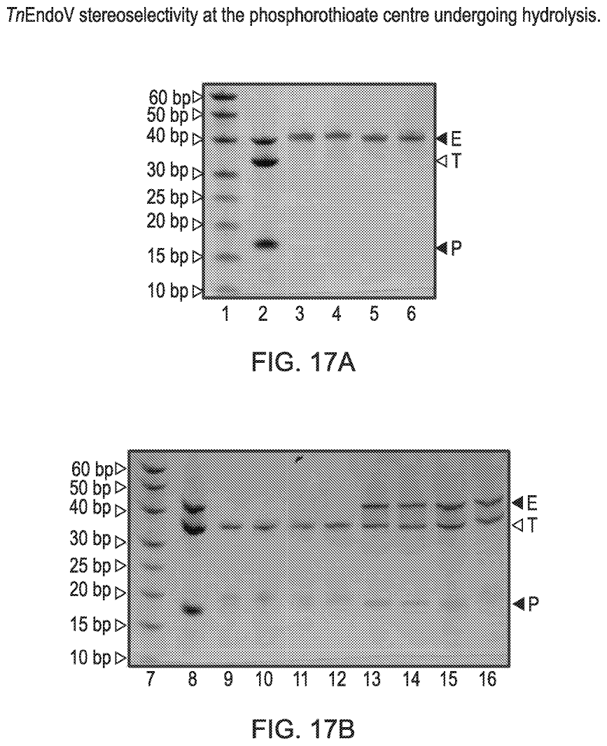

The stereochemistry was not controlled during the chemical synthesis of the extended template modified with phosphorothioate linkages (E6) resulting in a complex mixture of stereoisomers. EndoV catalysed cleavage of the phosphorothioate modified DNA yielded the expected product P6 (Table 1a) in ˜50% yield (FIG. 12 and FIG. 17) To test TnEndoV stereospecifity, extended DNA templates containing Rₚ-linkages (E11-E14) were produced enzymatically using KOD and Sₚ-dNTPaS. Extended templates were isolated using an oligonucleotide clean up kit (Monarch) and subsequently incubated with TnEndoV. PAGE analysis showed that the cleavage reactions proceeded to completion, suggesting that the endonucleases are capable of cleaving Rp phosphorothioate linkages (FIG. 17).

### MATERIALS AND METHODS

### Materials

All chemicals and biological materials were obtained from commercial suppliers. Kanamycin, ammonium persulfate, IPTG, N,N,N′, N′-tetramethylethylenediamine and boric acid were purchased from Sigma-Aldrich; LB agar, 2×YT media, arabinose and Tris base from Formedium; E. coli 5 alpha, Q5 DNA polymerase, T4 DNA ligase, restriction enzymes and ThermoPol reaction buffer from New England BioLabs; Escherichia coli BL21 (AI) and DH10B from ThermoFisher; Sₚ- and Rₚ-dNTPas, and Sp-2′F dNTPs from Biolog Life Science Institute; LNA-NTPs, 2′F-NTPs and dNTPaS (Sₚ/Rₚ 1:1) from Jena Bioscience; 2′MeO-NTPs from Trilink Biotechnologies; oligonucleotides were synthesized by Integrated DNA Technologies (IDT); acrylamide/bis-acrylamide 19:1 solution from Severn Biotech Ltd.; EDTA from National Diagnostics; Urea, formamide, and sodium dodecylsulfate from Fisher Scientific; Bromophenol blue from Alpha Aesar.

### Protein Production and Purification

The genes encoding polymerases and endonuclease Vs (endoVs) were codon optimized for expression in E. coli and ordered from IDT as g-blocks. TfPol* and MhPol* genes were cloned into pET28 using Ndel and Xhol restriction sites. All other genes were cloned into pET29 using Ndel and Xhol restriction sites. For expression of polymerases and EndoVs, chemically competent E. coli BL21 AI cells transformed with the appropriate plasmid DNA were used to inoculate 5 ml 2×YT medium containing 50 μg ml⁻¹ kanamycin. Following incubation at 37° C. for 18 hours, starter cultures (4 mL) were used to inoculate 400 ml 2×YT medium supplemented with 50 μg ml⁻¹ kanamycin. Cultures were incubated at 37° C., 200 r.p.m. until an optical density at 600 nm (OD₆₀₀) of 0.6. Protein expression was induced with the addition of IPTG (1 mM) and L-arabinose (3.33 mM), and cultures were incubated for 4 h at 37° C. Cells were pelleted by centrifugation (8000 r.p.m. for 20 min) and the supernatant was discarded. Cells were resuspended in lysis buffer (50 mM Hepes, 300 mM NaCI pH 7.5 containing 20 mM imidazole) and lysed by sonication. Cell lysates were heated to 70° C. for 30 minutes to denature endogenous proteins, then cleared by centrifugation (18,000 rpm for 20 minutes). His-tagged proteins were subjected to affinity chromatography using Ni-NTA Agarose (Qiagen) and eluted using 50 mM HEPES, 300 mM NaCI, pH 7.5 containing 250 mM imidazole. Purified proteins were desalted using 10DG desalting columns (Bio-Rad) and eluted with 2× storage buffer (20 mM Tris-HCI, 200 mM KCI, 0.2 mM EDTA, 2 mM DTT pH 8.0). Protein concentrations were determined at 280 nm using the extinction coefficient (Supplementary Table 1&2). One volume of glycerol was added to the aliquotted proteins prior to storage at −20° C.

### General Procedure for Polymerase Catalysed Extension Reactions (FIG. 4A)

To compare the activity of polymerases toward different modified NTPs, analytical scale biotransformations were performed using template (20 μM), dNTPs (0.25 mM) and polymerase (0.2 μM) in ThermoPol buffer (20 mM Tris-HCI, 10 mM (NH₄)₂SO_{4, 10} mM KCI, 2 mM MgSO₄, 0.1% Triton X-100, pH 8.8). Reactions were incubated at 70° C. for 12 hours unless otherwise stated and samples were analysed by HPLC, LC-MS or PAGE. To investigate sequential incorporation of modified NTPs, Templates T18-T21 were extended as described but using 1 mM modified NTP. Initial assays were performed as described but using a commercial KOD (Merck, 2.5 Units). General procedure for endonuclease catalysed cleavage reactions (FIG. 4B):

To compare the activity of EndoVs towards different modified oligonucleotides, analytical scale biotransformations were performed using extended template (20 μM) and EndoV (2 μM) in ThermoPol buffer (20 mM Tris-HCI, 10 mM (NH₄)₂SO₄, 10 mM KCI, 2 mM MgSO₄, 0.1% Triton X-100, pH 8.8). Reactions were incubated at 70° C. for 12 hours unless otherwise stated and samples were analysed by HPLC, LC-MS or PAGE. To investigate the effect of increased substrate (dNTP) loading on EndoV activity, biotransformations were performed as described with the addition of dNTPs (0-60 mM total). Initial assays were performed as described but using commercial TmEndoV (ThermoFisher, 5 units).

### General Procedure for One-Pot Polymerase-Endonuclease Reaction

To compare the effects of template sequence, analytical scale biotransformations were performed using template (20 μM), dNTPs (1 mM), KOD polymerase (0.2 μM) and TnEndoV (2 μM) in ThermoPol buffer (20 mM Tris-HCI, 10 mM (NH₄)₂SO₄, 10 mM KCI, 2 mM MgSO₄, 0.1% Triton X-100, pH 8.8). Reactions were incubated at 70° C. for 12 hours unless otherwise stated and samples were analysed by HPLC, LC-MS or PAGE. Initial assays were performed as described but using commercial KOD (Merck, 2.5 Units), TmEndoV (ThermoFisher, 5 units) and dNTPs (0.25 mM each). Reaction conditions for the synthesis of P19-P32 are reported in Table 4.

### Synthesis of PS Modified Oligonucleotides as Single Stereoisomers

One-pot KOD (0.1 μM) and TnEndoV (2 μM) reactions were performed using template T10-T13 (10 μM) three dNTPs (1.4 mM each), and one Sₚ-dNTPaS (0.7 mM) or Rp/Sp dNTPaS mix (0.7 mM). Reactions were incubated for 70° C. for 12 hours and analysed by HPLC in comparison with chemically synthesized standards.

### Optimized Conditions for P1 Synthesis

A range of buffers with varying compositions and concentrations were evaluated with respect to product yield. The following components were varied in the reaction: Buffer salts (Tris-HCI, Tris-AcOH, HEPES-KOH, Bicine-HCI, pH 8.0), metal ions (MgSO₄, MgCI₂, MgOAc₂, CaCI₂), amino acids (Glutamate, Arginine, Proline, Glycine, Serine, Aspartate, pH8) and additives (sodium citrate, (NH₄)₂SO₄, tetra-methylammonium chloride (TMAC), KCI, KOAc, glycerol, 1,2-propanol, sucrose, trehalose, acetylated BSA, Tween-20, Triton X-100, DTT, formamide, DMSO and PEG-3000). The optimized reaction conditions for KOD (2 μM) and TnEndoV (2 μM) catalyzed amplification of P1 using T1 (1 μM) and dNTPs (4 mM each), are 20 mM Tris-HCI (pH 8), 20 mM MgCl₂, 100 mM arginine-HCI (pH 8), 100 mM glutamate-KOH (pH 8), DTT (10 mM), formamide (10%), trehalose (0.2 M), 1,2-propanediol (1 M) and acetylated BSA (0.01 mg/ml). Under these conditions 330 cycles of template extension and product cleavage were achieved (FIG. 8)

### Preparative Scale Synthesis of P1

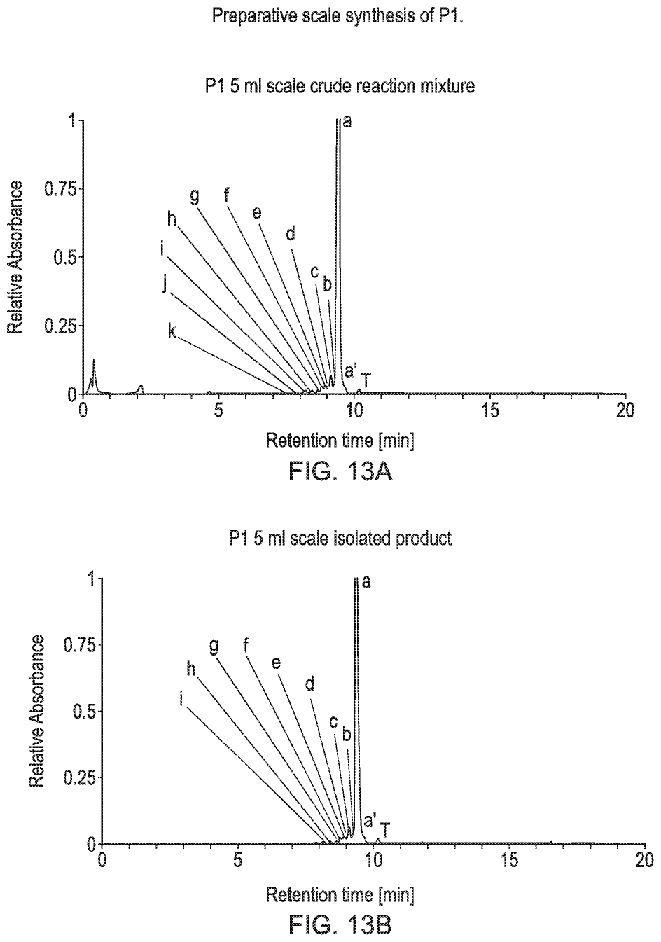

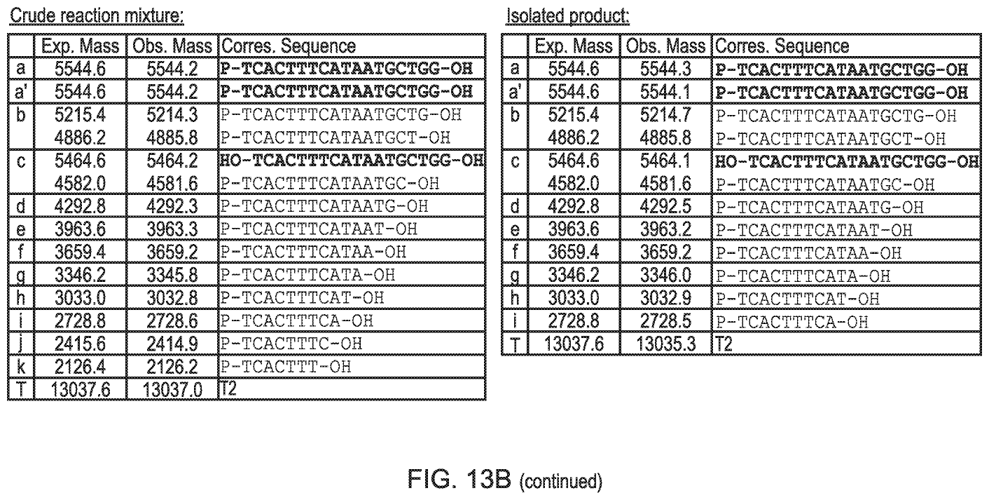

A 5 ml scale biotransformation was performed in the optimized reaction buffer (20 mM Tris HCI, 20 mM MgSO₄, 100 mM KGlu, 100 mM ArgHCI, 0.01 mg/ml AcBSA, 10 mM DTT, 10% formamide, 0.2 M trehalose, 1 M 1,2-propanediol pH 8.0), using dNTPs (4 mM each), T2 (4 μM), KOD (2 μM) and TnEndoV (2 μM). Following incubation at 70° C. for 12 hours the reaction was quenched with the addition of 0.5 M EDTA pH 8 (f.c. 40 mM). Proteins were denatured by heating to 98° C. for 2 hours and pelleted by centrifugation (15 min at 13,300 r.p.m.). The supernatant containing the oligonucleotides was collected and the protein pellet was washed with water (1000 μl). The combined aqueous fractions were extracted with 6 ml Tris-saturated phenol:chloroform:isoamyl solution (Sigma Aldrich) and washed with chloroform. Residual protein was removed using an 10K MWCO ultafiltration device (Sartorius). The flow-through was collected, concentrated, and desalted using a 3K MWCO ultafiltration device (Merck Millipore). The final product was freeze dried. FIG. 13).

Results: The final product P1 was achieved in 88% purity (11.5 mg) without chromatographic purification

### TnEndoV Stereoselectivity at the Phosphorothioate Centre Undergoing Hydrolysis

Stereodefined extended templates were synthesized using templates T14-T17 (20 μM), Sₚ-dNTPaS (0.25 mM each) and KOD (0.2 μM) in ThermoPol buffer (20 mM Tris-HCI, 10 mM (NH₄)₂SO₄, 10 mM KCI, 2 mM MgSO₄, 0.1% Triton X-100, pH 8.8). Reactions were incubated at 70° C. for 12 hours and samples were analysed by PAGE. The extended oligonucleotide products were purified using a Monarch 5 μg DNA Cleanup kit (New England Biolabs) according to the manufacturers protocol. The enzymatically synthesized oligonucleotides (20 μM) were incubated with TnEndoV (2 μM) in ThermoPol buffer at 70° C. for 12 hours. For comparison, chemically synthesized, stereorandom oligonucleotides were also incubated with TnEndoV as described. Samples were analysed by PAGE.

### Chromatographic Analysis

For chromatographic analysis, reactions were quenched with the addition of 20 mM EDTA, one volume of water was added and samples were heated at 98° C. for two hours to denature the proteins. Precipitated proteins were removed by centrifugation (14,000 g for 5 minutes) and the supernatant was transferred to a sample vial.

Ion-pairing reverse phase chromatography was performed using a 1290 Infinity II Agilent LC system with an AdvanceBio Oligonucleotides 2.7 μm column, 50×2.1 mm (Agilent) at 60° C. After a 2 minute hold at 5% buffer B, oligonucleotides were eluted over 10 minutes using a gradient of 5-50% Buffer B at 0.6 mL min⁻¹ followed by five minutes of re-equilibration to starting conditions before the next sample was injected. Peaks were assigned by comparison to chemically synthesized standards and the peak areas were integrated using Agilent OpenLab software. The relative molar extinction coefficient was calculated from reactions which proceeded to completion (Supplementary table 3) and these values were used to calculate the percentage conversion of incomplete reactions. For reactions involving phosphorothioate modifications the relative molar extinction coefficient for the corresponding phosphorylated products were used.

Buffer B: 100 mM triethyl ammonium acetate with 25% acetonitrile

Buffer A: 100 mM triethyl ammonium acetate

LC-MS analysis was performed using a Waters Vion IMS QTOF running in negative ESI mode at 2.2 kV capillary, acquiring up to 2000 m/z. This was coupled to a Waters Acquity I Class UPLC with a DNAPac™ RP 4 μm column, 50×2.1 mm (ThermoFisher) at 65° C. After a 2 min hold at 30% Buffer B, oligonucleotides were eluted over 8 minutes using a gradient of 30-80% Buffer B at 0.3 mL min-1 followed by five minutes of re-equilibration to starting conditions before the next sample was injected. The resulting multiply charged spectrum was analysed using Waters Unifi software and deconvoluted between 4000-20000 Da using the MaxEnt1 algorithm.

Buffer B: 400 mM hexafluoro isopropanol, 15 mM Triethylamine in water with 50% methanol

Buffer A: 400 mM hexafluoro isopropanol, 15 mM Triethylamine in water

### Analysis by Polyacrylamide Gel Electrophoresis (PAGE)

For analysis of oligonucleotides >18 nucleotides long, TBE-urea acrylamide gels were prepared containing 15% acrylamide/bis-acrylamide and 8 M urea in 1× TBE buffer (0.1 M Tris base, 0.1 M boric acid, 2 mM EDTA). For resolution of oligonucleotides <18 nucleotides, TBE-urea acrylamide gels were prepared containing 20% acrylamide/bis-acrylamide, 8 M urea, 1× TBE buffer (0.1 M Tris base, 0.1 M boric acid, 2 mM EDTA). Polymerisation was initiated by addition of ammonium persulfate (final conc. 0.1% w/v) and N,N,N′,N′-tetramethylethylenediamine (final conc. 4.7 μM). A sample volume of 2 μl was mixed with 2× denaturing RNA loading dye (95% v/v formamide, 0.02% w/v sodium dodecylsulfate, 1 mM EDTA, 0.02% w/v bromophenol blue) and water to give a final volume of 10 μl. Prior to loading, the samples were denatured at 95° C. for 1 min. Gels were run at room temperature in 1× TBE at 200 V for 50 minutes and visualised with 200 mg/L methylene blue in water.

### Effect of Increased Substrate (dNTP) Loading on Enzyme Activity (FIGS. 5A-C)

To investigate the effect of dNTP concentration on KOD activity, biotransformations were performed as described in the ‘General procedure for polymerase catalysed extension reactions’ but with varying dNTP concentrations (10-40 mM). To investigate the effect of dNTP concentration on TnEndoV and TmEndoV, reactions were performed as described in the ‘General procedure for endonuclease catalysed cleavage reactions’ but with the addition of varying concentrations of dNTPs (0-50 mM and 0-3 mM for TnEndoV and TmEndoV respectively). Samples were analysed by PAGE.

### Protein Thermal Shift Assay (FIG. 6A)

SYPRO Orange was added to protein (5 μM) in ThermoPol buffer (20 mM Tris-HCI, 10 mM (NH₄)₂SO₄, 10 mM KCI, 2 mM MgSO₄, 0.1% Triton X-100, pH 8.8) to a final concentration of 50×. Samples were incubated for 45 min at 25° C., then heated to 95° C. in 0.5° C. increments using a CFX96 Touch real time PCR (BioRad). The derivative of the fluorescence was calculated using CFX Maestro software.

### Effect of Reaction Temperature on One-Pot Polymerase-Endonuclease Reactions (FIG. 6B)

To investigate the effect of temperature on product yield, biotransformations were performed as described in the ‘General procedure for one-pot polymerase-endonuclease reactions’ but reactions were incubated at varying temperatures (60-85° C.)

### KOD and TnEndoV Temperature Profiles (FIGS. 6C-D)

To evaluate the thermostability of KOD and TnEndoV in the reaction, analytical scale biotransformations were performed using enzyme solutions pre-incubated for 0 hours or 24 hours at 70° C. in a thermocycler (BioRad). KOD (0.2 μM) catalysed extension reactions were performed using T1 (20 μM) and dNTPs (4 mM) in ThermoPol buffer. TnEndoV (2 μM) catalysed cleavage reactions were performed using E1 (20 μM) in ThermoPol buffer. Reactions were incubated at 70° C., samples were taken at 15 min, 30 min, 45 min and 1 hour, quenched with the addition of 20 μM EDTA and analysed by HPLC.

### Effect of Temperature Hairpin Sequence on One-Pot Polymerase-Endonuclease Reactions (FIG. 7)

To evaluate the effect of the hairpin sequence on product yield, biotransformations were performed as described in the ‘General procedure for one-pot polymerase-endonuclease reactions’ but using templates T1-T9 (20 μM).

### Optimized Procedure for One-Pot Polymerase-Endonuclease Reaction (FIG. 8)

Template (4 μM), dNTPs (16 mM), polymerase (0.2 μM) and EndoV (2 M) in Buffer (20 mM Tris pH 8, 50 mM KCI, 12 mM MgSO₄, 10 mM DTT, 0.1% BSA, 100 mM Arginine, 100 mM glutamate) was incubated at 70° C. for 12 hours. A sample was taken and analysed by HPLC.

### Time-Resolved FRET Based Polymerase Assay (FIG. 9)

Assays were performed in black 384-well plates (Greiner) containing FRET template (400 nM), FRET primer (500 nM) and dNTPs (2.5 mM each) in Thermopol buffer (15 μL).

Centrifugation at 4000 rpm, for 2 minutes removed any bubbles. Samples were incubated at 37° C. for 20 minutes then reactions were initiated with the addition of polymerase (5 ul, 0.25 μM final concentration). Fluorescence was measured with excitation at 485 nm and emission at 518 nm, on a Clariostar plate reader (BMG) over 60 minutes. Initial reaction rates were measured in triplicate and the rate of reaction towards modified substrates are reported relative to the activity with natural dNTPs.

### Stereoselective Synthesis of Oligonuleotides Containing a Single Phosphorothioate Linkage (FIG. 10)

One pot reactions were performed using KOD (0.1 μM), TnEndoV (2 μM), 10 μM template and a 5 mM dNTP mix containing 3 natural dNTPs and one dNTPaS (Sₚ-dNTP or a Rₚ/Sₚ-dNTP mix). Reactions were incubated for 70° C. for 12 hours and then analysed by HPLC.

### Substrate Profiling of the EndonucleaseV Panel (FIGS. 11 and 12)

Analytical scale biotransformations were performed using extended templates (20 μM) and EndoV (5 μM) in ThermoPol buffer (20 mM Tris-HCI, 10 mM (NH₄)₂SO₄, 10 mM KCI, 2 mM MgSO₄, 0.1% Triton X-100, pH 8.8) and reactions were incubated at 70° C. Samples were taken at 1 and 18 hours and quenched with the addition of 20 mM EDTA. Samples were analysed by PAGE, HPLC and LC-MS analysis.

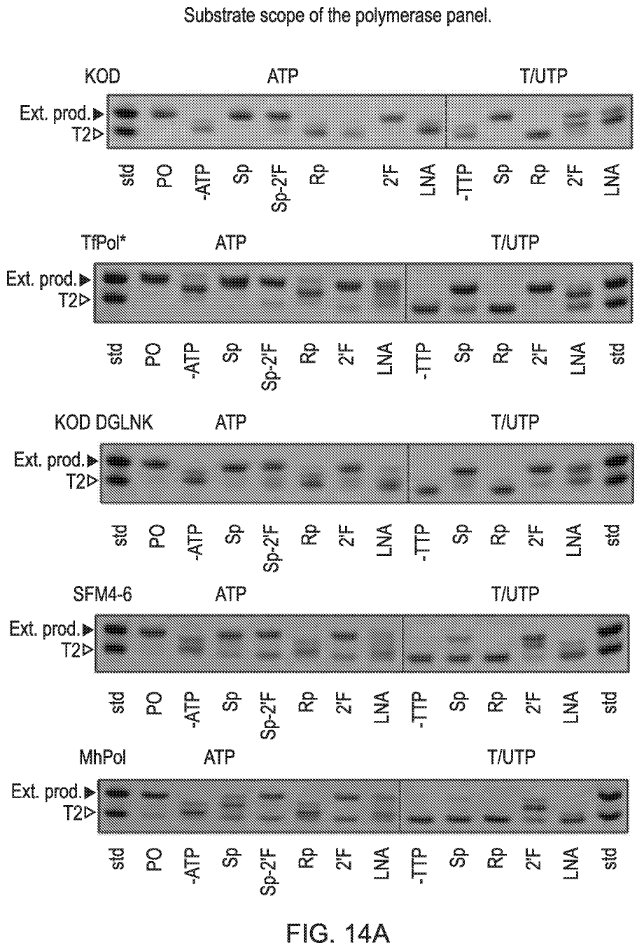

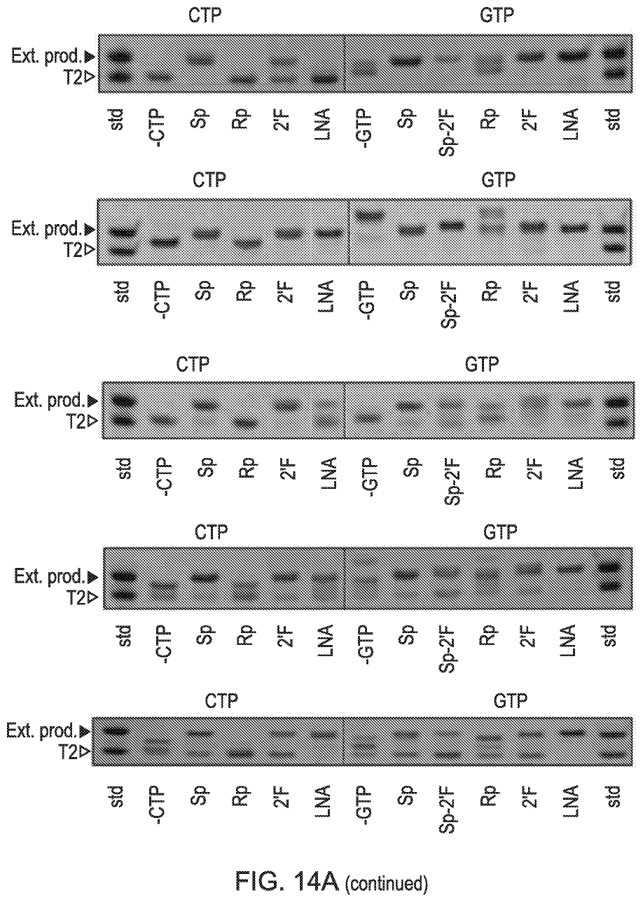

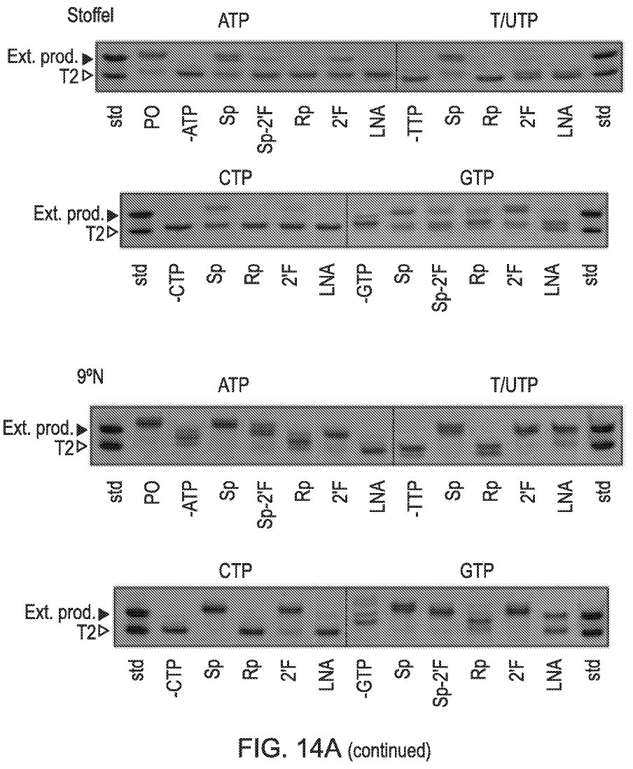

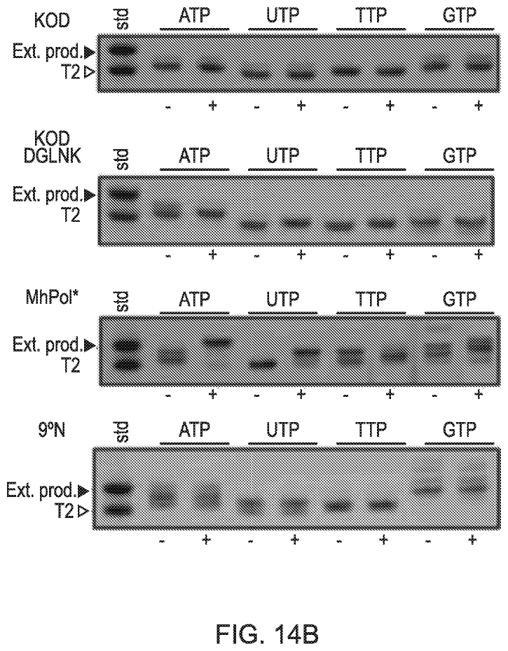

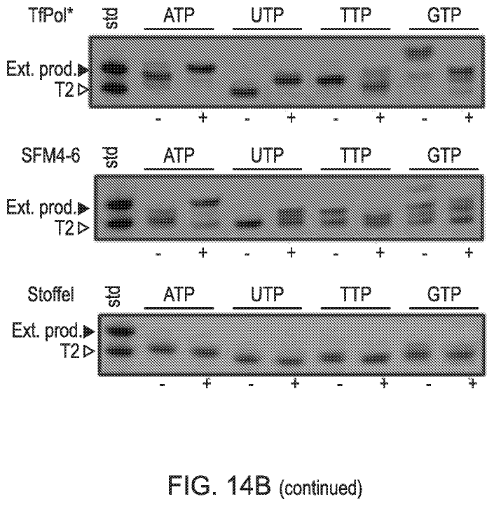

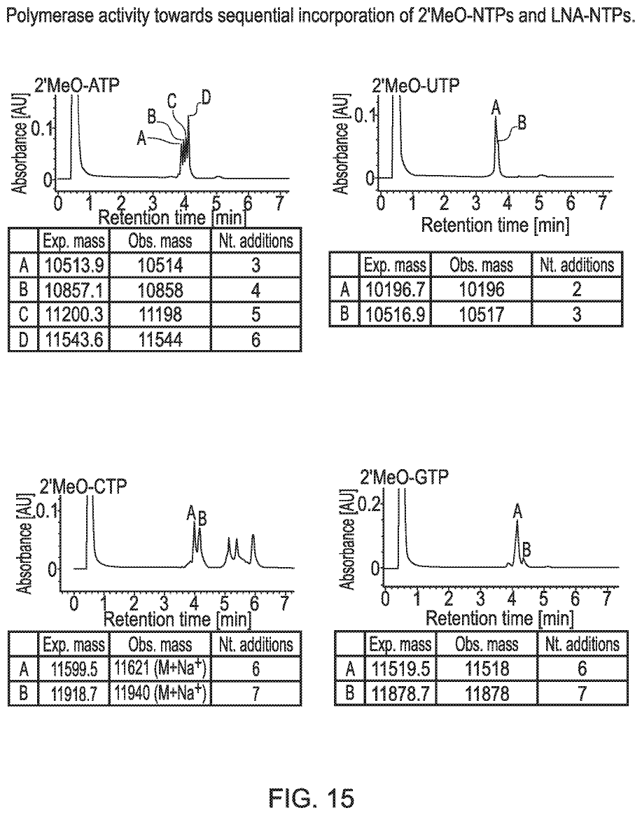

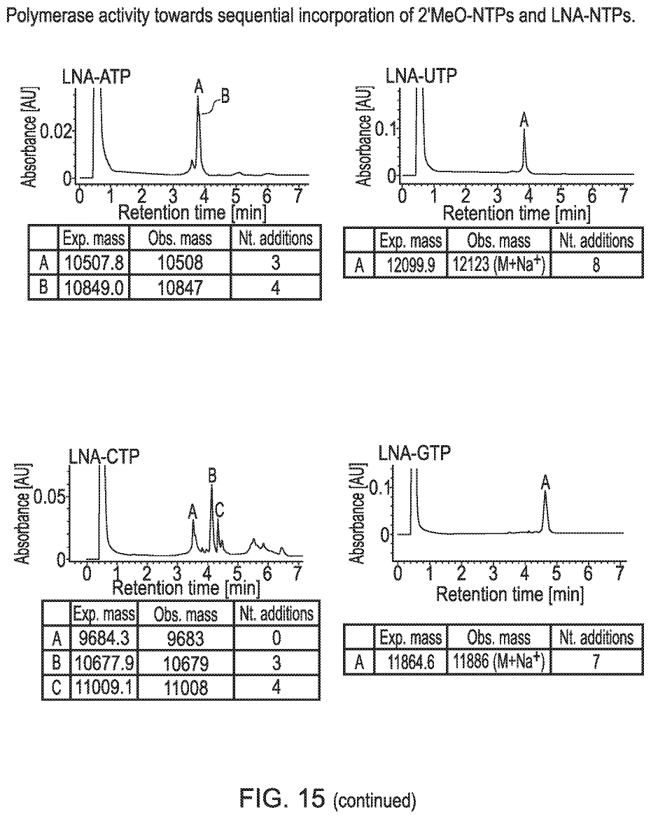

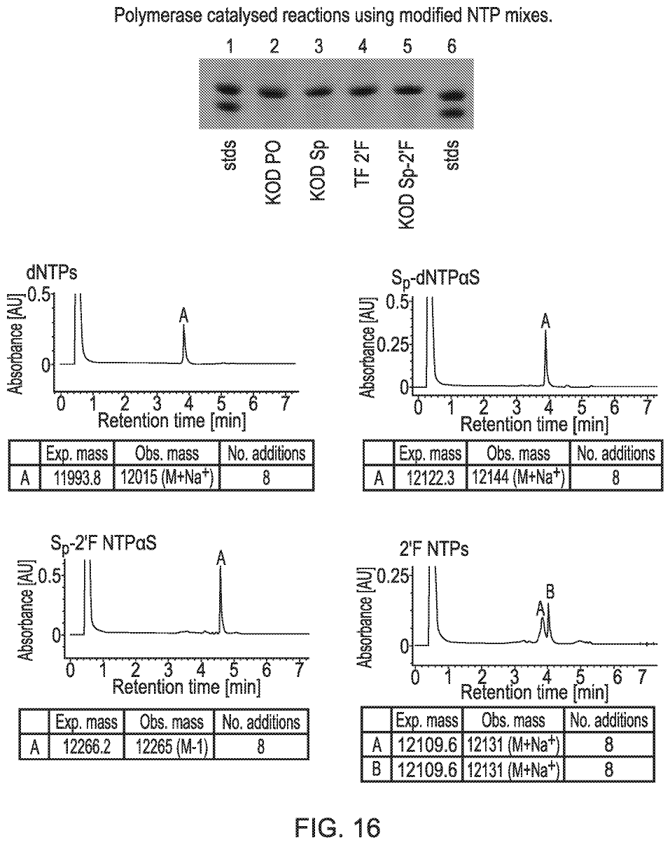

### Substrate Profiling of Polymerases (FIGS. 14 and 15)

To compare the activity of polymerases towards different modified NTPs, analytical scale biotransformations were performed using templates T18-T21 (20 μM), NTPs (0.25 mM each) and polymerase (0.2 μM) in ThermoPol buffer (20 mM Tris-HCI, 10 mM (NH₄)₂SO₄, 10 mM KCI, 2 mM MgSO₄, 0.1% Triton X-100, pH 8.8). Reactions were incubated at 70° C. and samples were analyzed by LC-MS and denaturing urea PAGE.

Results: Polymerases with activity towards phosphorothioate, 2′-fluoro, 2′-methoxy, and locked nucleic acid modified NTPs have been identified

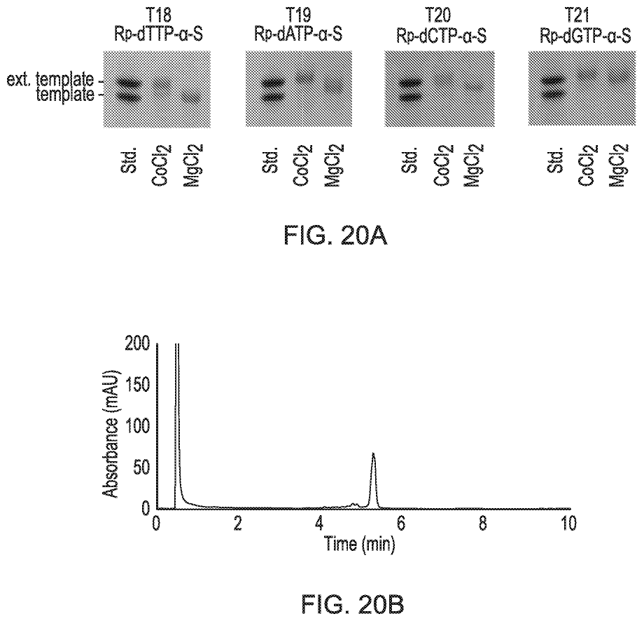

### Polymerase Activity Towards Rp-dNTPaS Using Cobalt And Manganese Cofactors (FIG. 20)

Analytical scale extension reactions were performed using template T18-T21 (10 μM) and KOD (6 μM) in 20 mM Tris-HCI pH 8, 10 mM KCI, 10 mM (NH₄)₂SO₄, the corresponding Rp-dNTPaS with CoCI₂ or MgCl₂ salts. Reactions were incubated at 70° C. for 12 hours and samples were analysed denaturing urea PAGE and LC-MS analysis.

Results: In the presence of CoCI₂, KOD can accept Rp-dNTPaS building blocks

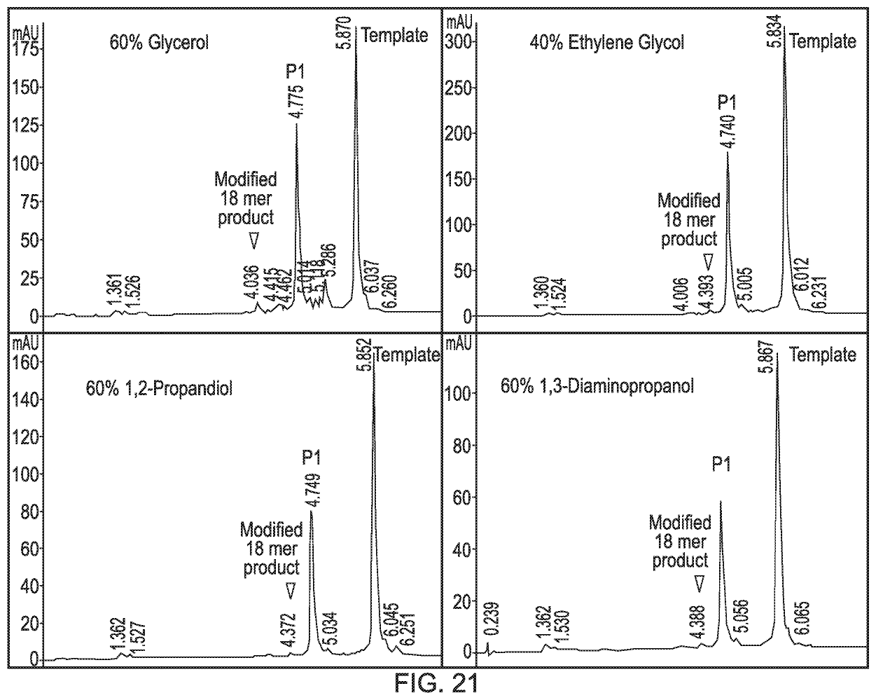

### Endonuclease Catalyzed Cleavage Using Alternative Nucleophiles to Water (FIG. 21)

Analytical scale biotransformations were performed using TnEndoV (2 μM) and extended template E1 (20 μM) in ThermoPol buffer (20 mM Tris-HCI, 10 mM (NH₄)₂SO₄, 10 mM KCI, 2 mM MgSO₄, 0.1% Triton X-100, pH 8.8), with the addition of 60% w/v glycerol, 40% w/v ethylene glycol, 60% w/v 1,2-propandiol or 60% w/v 1,3-diaminopropanol. Reactions were incubated at 70° C. for 2 hours and then analysed by LC-MS.

Results: MS analysis showed that in the presence of high concentrations of nucleophile, EndoV catalyzed the addition of the alternative nucleophile to the 5′-phosphate group of the 18 mer product. This provided a modified oligonucleotides suitable for conjugation to delivery vehicles such as antibodies, peptides, proteins or sugars for targeted delivery to specific organs. Although the modified 18 mer is a minor product, the yield can be improved by engineering the EndoV using directed evolution.

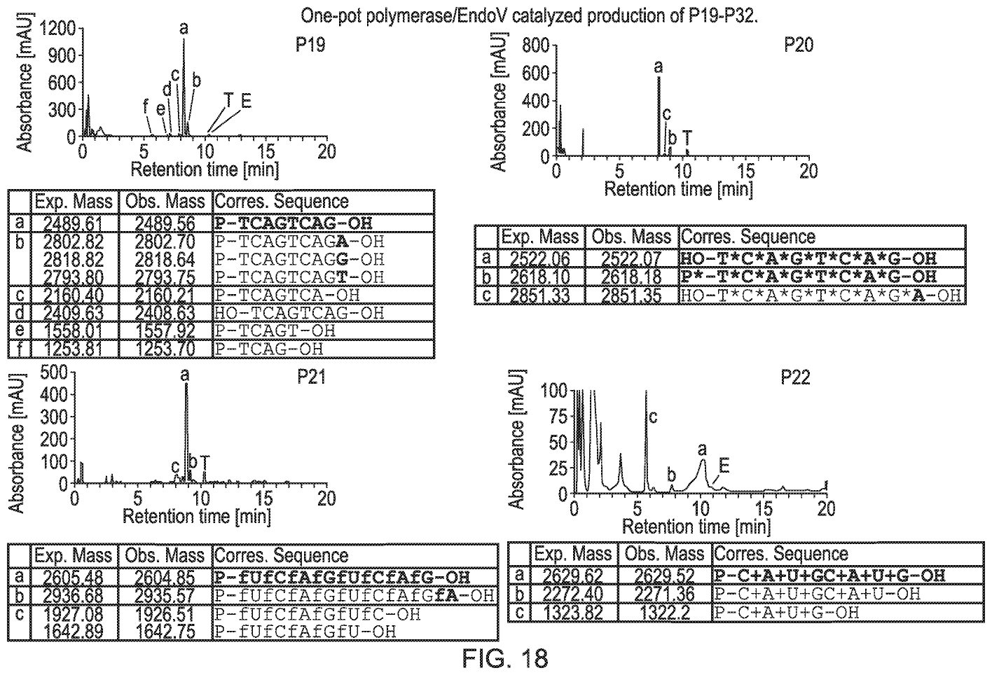

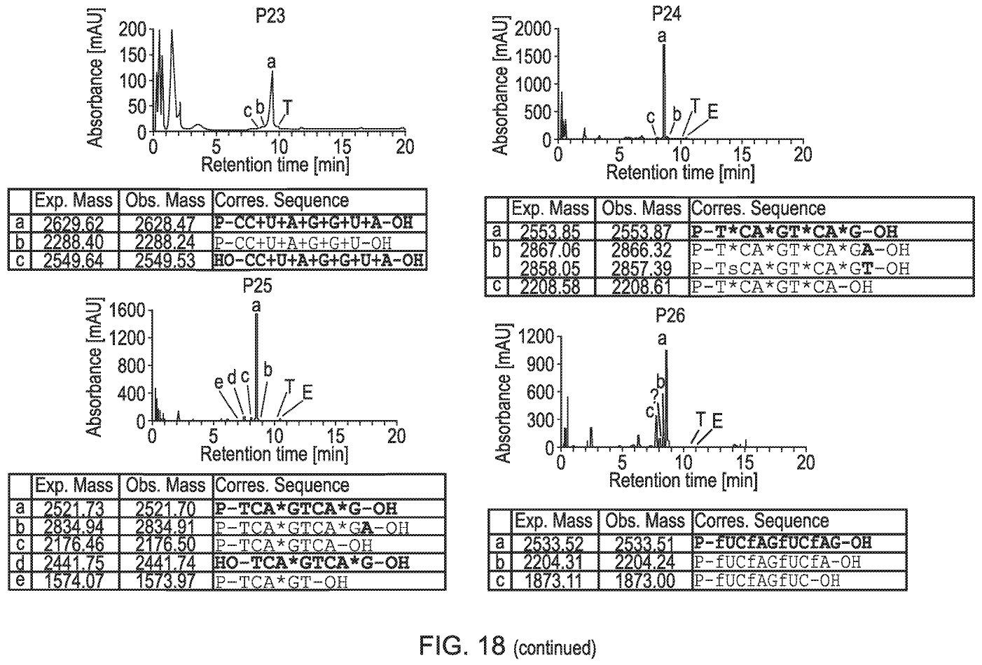

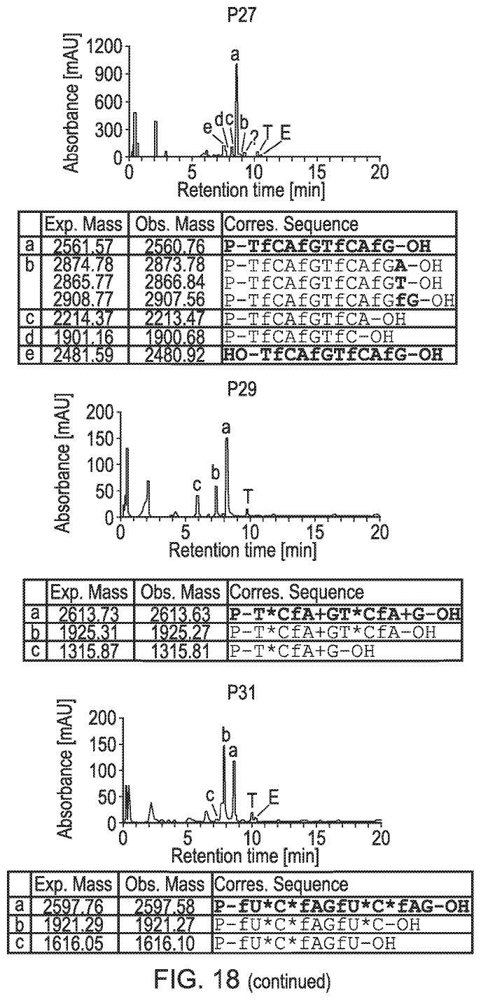

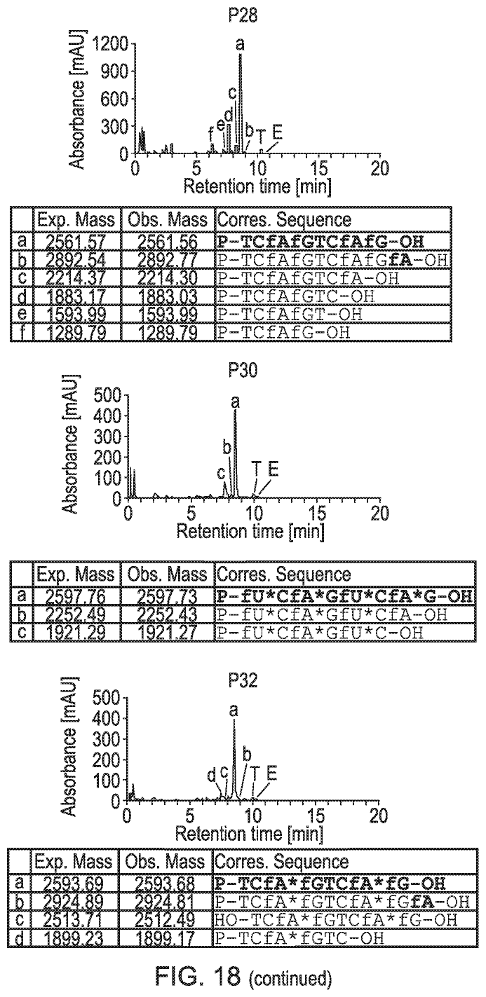

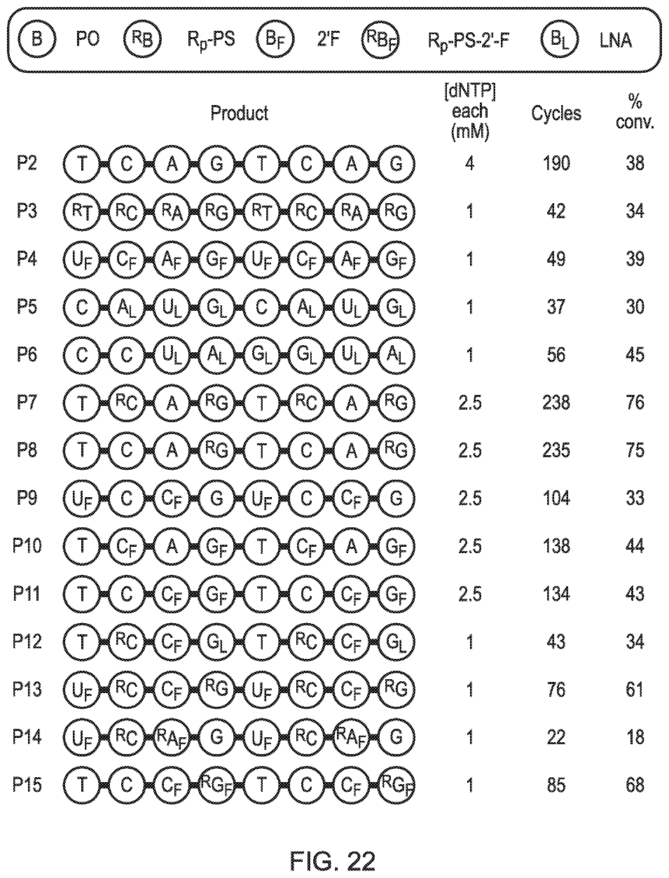

### General Protocol for One-Pot Polymerase/EndoV Catalyzed Oligonucleotide Synthesis (FIGS. 18 and 22)

Analytical scale biotransformations were performed using template (20 μM), dNTPs (1 mM each), KOD (0.2 μM) and TnEndoV (2 M) in ThermoPol buffer (20 mM Tris-HCI, 10 mM (NH₄)₂SO₄, 10 mM KCI, 2 mM MgSO₄, 0.1% Triton X-100, pH 8.8). Reactions were incubated at 70° C. for 12 hours and samples were analyzed by LC-MS and denaturing urea PAGE. Reaction conditions for the synthesis of P19-P32 are reported in supplementary table 3.

Results: A range of 8 mer oligonucleotide products with different base sequences and a variety of 2′F, Rₚ-PS, Rₚ-2′F-PS and LNA modifications were successfully amplified. In favourable cases, up to 238 cycles of template extension and product cleavage were achieved, consuming 76% of the NTP starting materials

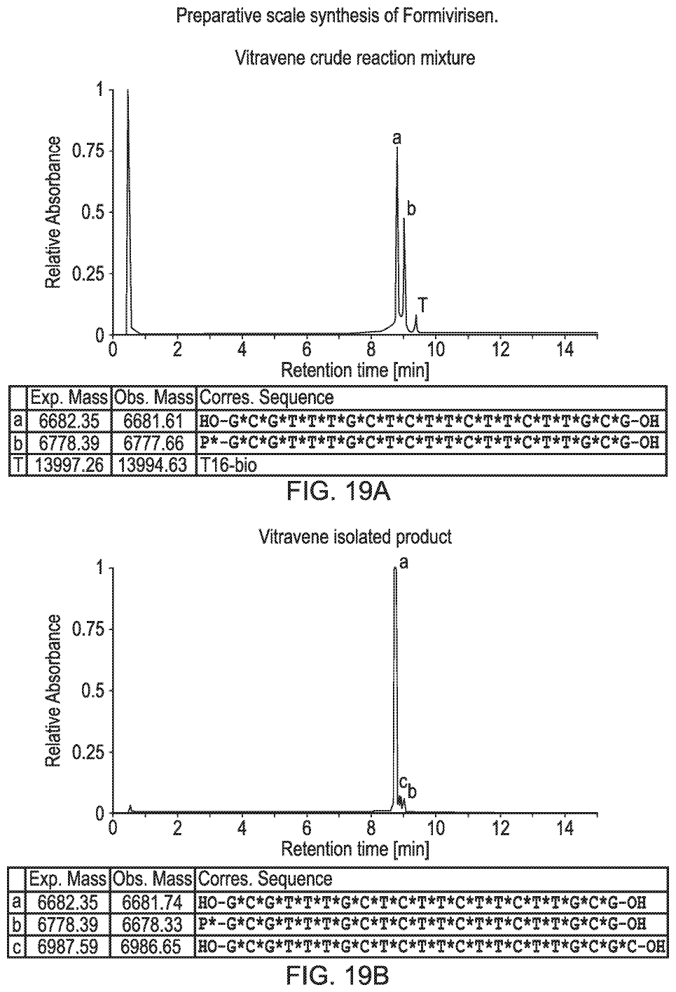

### Preparative Scale Synthesis of Vitravene (FIG. 19)

A 0.75 ml scale biotransformation was performed in buffer containing 50 mM Tris AcOH, 20 mM MgSO₄, 50 mM KOAc, 100 mM KGlu, 100 mM ArgHCI, 0.01 mg/ml AcBSA, 0.1% Triton X-100, 30 mM DTT, 5% formamide, pH 8.0, using Sₚ-dGTPaS (1.4 mM), Sₚ-dCTPaS (1.7 mM), Sₚ-dTTPaS (2.9 mM), template T25 (4 μM), KOD (4 μM) and TnEndoV (4 μM). Following incubation at 70° C. for 12 hours, the reaction was quenched by heating to 98° C. for 2 h. The reaction mixture was desalted on a 1K MWCO Microsep Advance (Pall). The template was removed by addition of DNAse I (f.c. 60 U/ml) (New England Biolabs) and DNAse I 10× buffer (f.c. 1×) and incubated at 37° C. for 1 h. DNAse I was heat inactivated at 75° C. for 10 min and the reaction mixture was again desalted on a 1K MWCO Microsep Advance. Subsequently, 5′ terminal phosphate groups were removed by addition of quick CIP (f.c. 250 U/ml) (New England Biolabs) and 10× quickCIP buffer (f.c. 1×), followed by incubation for 3 h at 37° C. Proteins were heat denatured by incubating the sample at 95° C. for 20 min and precipitated proteins were pelleted by centrifugation (15 min at 13,300 r.p.m.). The supernatant containing the oligonucleotides was collected and the protein pellet was washed with water (200 μl). The combined aqueous fractions were extracted with Tris-saturated phenol-chlorofrom-isoamyl solution (Sigma Aldrich) and washed with chloroform to remove any remaining protein. Residual traces of organic impurities and salts were removed by washing the product on a 1K MWCO Microsep Advance. The final product was freeze dried.

Results: The final product was produced (0.26 mM) following 65 cycles of template extension and product cleavage, which correlates to consumption of 90% of the available Sₚ-dNTPaS starting materials. The final product was isolated in 87% purity without chromatographic purification.

### SEQUENCES

TABLE 1a
Oligonucleotide sequences

| SEQ NO | | Sequence |
|---|---|---|
| SEQ ID NO: 1 | T1 | cca gca tta tga aag tga cac gtg cac cat tgg tgc acg /ideoxyl/g |
| SEQ ID NO: 2 | E1 | cca gca tta tga aag tga cac gtg cac cat tgg tgc acg /ideoxyl/g tca ctt |
| | | tca taa tgc tgg |
| SEQ ID NO: 3 | P1 | (p) tca ctt tca taa tgc tgg |
| SEQ ID NO: 4 | T2 | Biotin-cca gca tta tga aag tga cac gtg cac cat tgg tgc acg /ideoxyl/g |
| SEQ ID NO: 5 | T3 | cca gca tta tga aag tga cac gtg cac cat-(biotin) tgg tgc acg /ideoxyl/g |
| SEQ ID NO: 6 | T4 | cca gca tta tga aag tga cac gtg cac ca tgg tgc acg /ideoxyl/g |
| SEQ ID NO: 7 | T5 | cca gca tta tga aag tga cac ggg cgc cgt cgg cgc ccg /ideoxyl/g |
| SEQ ID NO: 8 | T6 | cca gca tta tga aag tga ctc gtg cac cat tgg tgc acg /ideoxyl/g |
| SEQ ID NO: 9 | T7 | cca gca tta tga aag tga ccc gtg cac cat tgg tgc acg /ideoxyl/g |
| SEQ ID NO: 10 | T8 | cca gca tta tga aag tga cac gtg ca att tgc acg /ideoxyl/g |
| SEQ ID NO: 11 | T9 | cca gca tta tga aag tga cac gtg cac gc cat tgg cg tgc acg /ideoxyl/g |
| SEQ ID NO: 12 | FRET | CGT CGA CG/i5-TAMK/ AGG AGG AAA GGA CAT CTT CTA GCA /iFluorT/AC |
| | temp- | GTC GAC GAT GCA TGC ACT GGT CAG AAT TCG CAC CAC |
| | late | |
| SEQ ID NO: 13 | FRET | GTG GTG CGA ATT CTG AC |
| | primer | |
| SEQ ID NO: 14 | T10 | agc t agc cac gtg cac caa tgg tgc acg /ideoxyl/g |
| SEQ ID NO: 15 | T11 | cta g cta cac gtg cac caa tgg tgc acg /ideoxyl/g |
| SEQ ID NO: 16 | T12 | gct a gct cac gtg cac caa tgg tgc acg /ideoxyl/g |
| SEQ ID NO: 17 | T13 | tga c ga cac gtg cac caa tgg tgc acg /ideoxyl/g |
| SEQ ID NO: 18 | P2 | (p) gct*agct |
| SEQ ID NO: 19 | P3 | (p) tag*ctag |
| SEQ ID NO: 20 | P4 | (p) agc*tagc |
| SEQ ID NO: 21 | P5 | (p) tc*gtca |
| SEQ ID NO: 22 | E2 | cca gca tta tga aag tga cac gtg cac cat tgg tgc acg/ideoxyl/g Uf Cf Af |
| | | Cf Uf Uf Uf Cf Af Uf Af Af Uf Gf Cf Uf Gf Gf |
| SEQ ID NO: 23 | P6 | (p) Uf Cf Af Cf Uf Uf Uf Cf Af Uf Af Af Uf Gf Cf Uf Gf Gf |
| SEQ ID NO: 24 | E3 | cca gca tta tga aag tga cac gtg cac cat tgg tgc acg/ideoxyl/g Um Cm Am |
| | | Cm Um Um Um Cm Am Um Am Am Um Gm Cm Um Gm Gm |
| SEQ ID NO: 25 | P7 | (p) Um Cm Am Cm Um Um Um Cm Am Um Am Am Um Gm Cm Um Gm Gm |
| SEQ ID NO: 26 | E4 | cca gca tta tga aag tga cac gtg cac cat tgg tgc acg/ideoxyl/g T_{M} C_{M} A_{M} |
| | | C_{M} T_{M} T_{M} T_{M} C_{M} A_{M} T_{M} A_{M} A_{M} T_{M} G_{M} C_{M} T_{M} G_{M} G_{M} |
| SEQ ID NO: 27 | P8 | (p) T_{M} C_{M} A_{M} C_{M} T_{M} T_{M} T_{M} C_{M} A_{M} T_{M} A_{M} A_{M} T_{M} G_{M} C_{M} T_{M} G_{M} G_{M} |
| SEQ ID NO: 28 | E5 | ca gca tta tga aag tga cac gtg cac cat tgg tgc acg/ideoxyl/g +T C A C T |
| | | T T C A T A A T G C T G G |
| SEQ ID NO: 29 | P9 | (p) +T C A C T T T C A T A A T G C T G G |
| SEQ ID NO: 30 | T14 | ca gca tta tga aag tga cac gtg cac cat tgg tgc acg/ideoxyl/g |
| SEQ ID NO: 31 | E6 | cca gca tta tga aag tga cac gtg cac cat tgg tgc acg/ideoxyl/g *T *C *A |
| | | *C *T *T *T *C *A *T *A *A *T *G *C *T *G *G |
| SEQ ID NO: 32 | P10 | *T *C *A *C *T *T *T *C *A *T *A *A *T *G *C *T *G *G |
| SEQ ID NO: 33 | E7 | cca gca tta tga aag tga cac gtg cac cat tgg tgc acg/ideoxyl/g *T C A C |
| | | T T T C A T A A T G C T G G |
| SEQ ID NO: 34 | E8 | cca gca tta tga aag tgt cac gtg cac cat tgg tgc acg/ideoxyl/g *A C A C |
| | | T T T C A T A A T G C T G G |
| SEQ ID NO: 35 | E9 | cca gca tta tga aag tgg cac gtg cac cat tgg tgc acg/ideoxyl/g *C C A C |
| | | T T T C A T A A T G C T G G |
| SEQ ID NO: 36 | E10 | cca gca tta tga aag tgc cac gtg cac cat tgg tgc acg/ideoxyl/g *G C A C |
| | | T T T C A T A A T G C T G G |
| SEQ ID NO: 37 | E11 | cca gca tta tga aag tga cac gtg cac cat tgg tgc acg/ideoxyl/g *^{Rp}T C A |
| | | C *^{Rp}T *^{Rp}T *^{Rp}T C A *^{Rp}T A A *^{Rp}T G C *^{Rp}T G G |
| SEQ ID NO: 38 | E12 | cca gca tta tga aag tga cac gtg cac cat tgg tgc acg/ideoxyl/g *^{Rp}A C *^{Rp}A |
| | | C T T T C *^{Rp}A T *^{Rp}A *^{Rp}A TG CTG G |
| SEQ ID NO: 39 | E13 | cca gca tta tga aag tga cac gtg cac cat tgg tgc acg/ideoxyl/g *^{Rp}C *^{Rp}C |
| | | A *^{Rp}C T T T *^{Rp}C A T A A T G *^{Rp}C T G G |
| SEQ ID NO: 40 | E14 | cca gca tta tga aag tga cac gtg cac cat tgg tgc acg/ideoxyl/g *RPG C A |
| | | C T T T C A T A A T *^{Rp}G C T *^{Rp}G *^{Rp}G |
| SEQ ID NO: 41 | T15 | cca gca tta tga aag tgt cac gtg cac cat tgg tgc acg/ideoxyl/g |
| SEQ ID NO: 42 | T16 | cca gca tta tga aag tgg cac gtg cac cat tgg tgc acg/ideoxyl/g |
| SEQ ID NO: 43 | T17 | cca gca tta tga aag tgc cac gtg cac cat tgg tgc acg/ideoxyl/g |
| SEQ ID NO: 44 | P11 | *T C A C T T T C A T A A T G C T G G |
| SEQ ID NO: 45 | P12 | *A C A C T T T C A T A A T G C T G G |
| SEQ ID NO: 46 | P13 | *C C A C T T T C A T A A T G C T G G |
| SEQ ID NO: 47 | P14 | *G C A C T T T C A T A A T G C T G G |
| SEQ ID NO: 48 | P15 | *^{Rp}T C A C *^{Rp}T *^{Rp}T *^{Rp}T C A *^{Rp}T A A *^{Rp}T G C *^{Rp}T G G |
| SEQ ID NO: 49 | P16 | *^{Rp}A C *^{Rp}A C T T T C *^{Rp}A T *^{Rp}A *^{Rp}A TG C T G G |
| SEQ ID NO: 50 | P17 | *^{Rp}C *^{Rp}C A *^{Rp}C T T T *^{Rp}C A T A A T G *^{Rp}C T G G |
| SEQ ID NO: 51 | P18 | *^{Rp}G C A C T T T C A T A A T *^{Rp}G C T *^{Rp}G *^{Rp}G |

(p) = 5′-phosphate
Biotin = 5′-biotin
t-(biotin) = Biotin-modified thymidine residues
i5-TAMK = 5-isomer of TAMRA ™
iFluorT = Fluorescein is attached to position 5 of the thymine ring by a 6-carbon spacer arm
/ideoxyl/ = deoxyinosine
* = 5′-phosphorothioate linkage
*^{Rp} = 5′-Rₚ phosphorothioate linkage
*^{Sp} = 5′-Sₚ phosphorothioate linkage
Additional Oligonucleotide sequences

| SEQ ID | | Oligonucleotide Sequence |
|---|---|---|
| SEQ ID NO: 52 | T18 | 5′-AAA AAA AAC ACG TGC ACC ATT GGT GCA CGI G-3′ |
| SEQ ID NO: 53 | T19 | 5′-TTT TTT TTC ACG TGC ACC ATT GGT GCA CGI G-3′ |
| SEQ ID NO: 54 | T20 | 5′-GGG GGG GGC ACG TGC ACC ATT GGT GCA CGI G-3′ |
| SEQ ID NO: 55 | T21 | 5′-CCC CCC CCC ACG TGC ACC ATT GGT GCA CGI G-3′ |
| SEQ ID NO: 56 | T22 | 5′-CTG ACT GAC ACG TGC ACC ATT GGT GCA CGI G-3′ |
| SEQ ID NO: 57 | T23 | 5′-CAT GCA TGC ACG TGC ACC ATT GGT GCA CGI G-3′ |
| SEQ ID NO: 58 | T24 | 5′-TAC CTA GGC ACG TGC ACC ATT GGT GCA CGI G-3′ |
| SEQ ID NO: 59 | T25 | 5′-(Bio)CGC AAG AAG AAG AGC AAA CGC CAC GTG CAC CAT TGG |
| | | TGC ACG IG-3′ |
| SEQ ID NO: 60 | P19 | 5′-(P)TCA GTC AG-3′ |
| SEQ ID NO: 61 | P20 | 5′-(P)*T*C*A*G*T*C*A*G-3′ |
| SEQ ID NO: 62 | P21 | 5′-(P)fU fC fA fG fU fC fA Fg-3′ |
| SEQ ID NO: 63 | P22 | 5′-(P)C +A +U +G C +A +U +G-3′ |
| SEQ ID NO: 64 | P23 | 5′-(P)C C +U +A +G +G +U +A-3′ |
| SEQ ID NO: 65 | P24 | 5′-(P)T *C A *G T *C A *G-3′ |
| SEQ ID NO: 66 | P25 | 5′-(P)T CA *G T C A *G-3′ |
| SEQ ID NO: 67 | P26 | 5′-(P) fU C fA G fU C fA G-3′ |
| SEQ ID NO: 68 | P27 | 5′-(P)T fC A fG T fC A fG-3′ |
| SEQ ID NO: 69 | P28 | 5′-(P)T C fA fG T C fA fG-3′ |
| SEQ ID NO: 70 | P29 | 5′-(P)T *C fA +G T *C fA +G-3′ |
| SEQ ID NO: 71 | P30 | 5′-(P) fU *C fA *G fU *C fA *G-3′ |
| SEQ ID NO: 72 | P31 | 5′-(P) fU *C *fA G fU *C *fA G-3′ |
| SEQ ID NO: 73 | P32 | 5′-(P)T C A *fG T C A *fG-3′ |
| SEQ ID NO: 74 | Vit- | 5′ GCG TTT GCT CTT CTT CTT GCG-3′ |
| | ravene | |

(P): 5′ phosphate group
I: 2′-deoxyinosine
(Bio): 5′-biotinylated (IDT code: /5Biosg/)
m: 2′-OMe
+: LNA
Me: 2′-methoxyethoxy
MeC: 2′-methoxyethoxy ribose-5′-methylcytidine
*: phosphorothioate linkage
f: 2′-fluoro

TABLE 3
Polymerase Sequences

KOD (SEQ ID NO: 75)
Polymerase from Thermococcus kodakaraensis with mutations in the proof reading domain (D141A
E143A) Extinction coefficient: 125,030 M⁻¹cm⁻¹ Mass: 91.0 kDa
MILDTDYITEDGKPVIRIFKKENGEFKIEYDRTFEPYFYALLKDDSAIEEVKKITAERHGTVVTVKRVE
KVQKKFLGRPVEVWKLYFTHPQDVPAIRDKIREHPAVIDIYEYDIPFAKRYLIDKGLVPMEGDEELKM
LAFAIATLYEEGEEFAEGPILMISYADEEGARVITWKNVDLPYVDVVSTEREMIKRFLRVVKEKDPDV
LITYNGDNFDFAYLKKRCEKLGINFALGRDGSEPKIQRMGDRFAVEVKGRIHFDLYPVIRRTINLPTY
TLEAVYEAVFGQPKEKVYAEEITTAWETGENLERVARYSMEDAKVTYELGKEFLPMEAQLSRLIGQ
SLWDVSRSSTGNLVEWFLLRKAYERNELAPNKPDEKELARRRQSYEGGYVKEPERGLWENIVYLD
FRSLYPSIIITHNVSPDTLNREGCKEYDVAPQVGHRFCKDFPGFIPSLLGDLLEERQKIKKKMKATID
PIERKLLDYRQRAIKILANSYYGYYGYARARWYCKECAESVTAWGREYITMTIKEIEEKYGFKVIYSD
TDGFFATIPGADAETVKKKAMEFLKYINAKLPGALELEYEGFYERGFFVTKKKYAVIDEEGKITTRGL
EIVRRDWSEIAKETQARVLEALLKDGDVEKAVRIVKEVTEKLSKYEVPPEKLVIHEQITRDLKDYKAT
GPHVAVAKRLAARGVKIRPGTVISYIVLKGSGRIGDRAIPFDEFDPTKHKYDAEYYIENQVLPAVERIL
RAFGYRKEDLRYQKTRQVGLSAWLKPKGTLEHHHHHH*
90N (SEQ ID NO: 76)
Polymerase from Thermococcus sp. 9ON-7 with mutations in the proof reading domain (D141A
E143A) Extinction coefficient: 122,050 M⁻¹cm⁻¹ Mass: 90.8 kDa
MILDTDYITENGKPVIRVFKKENGEFKIEYDRTFEPYFYALLKDDSAIEDVKKVTAKRHGTVVKVKRA
EKVQKKFLGRPIEVWKLYFNHPQDVPAIRDRIRAHPAVVDIYEYDIPFAKRYLIDKGLIPMEGDEELT
MLAFAIATLYHEGEEFGTGPILMISYADGSEARVITWKKIDLPYVDVVSTEKEMIKRFLRVVREKDPD
VLITYNGDNFDFAYLKKRCEELGIKFTLGRDGSEPKIQRMGDRFAVEVKGRIHFDLYPVIRRTINLPT
YTLEAVYEAVFGKPKEKVYAEEIAQAWESGEGLERVARYSMEDAKVTYELGREFFPMEAQLSRLIG
QSLWDVSRSSTGNLVEWFLLRKAYKRNELAPNKPDERELARRRGGYAGGYVKEPERGLWDNIVY
LDFRSLYPSIIITHNVSPDTLNREGCKEYDVAPEVGHKFCKDFPGFIPSLLGDLLEERQKIKRKMKAT
VDPLEKKLLDYRQRLIKILANSFYGYYGYAKARWYCKECAESVTAWGREYIEMVIRELEEKFGFKVL
YADTDGLHATIPGADAETVKKKAKEFLKYINPKLPGLLELEYEGFYVRGFFVTKKKYAVIDEEGKITT
RGLEIVRRDWSEIAKETQARVLEAILKHGDVEEAVRIVKEVTEKLSKYEVPPEKLVIHEQITRDLRDY
KATGPHVAVAKRLAARGVKIRPGTVISYIVLKGSGRIGDRAIPADEFDPTKHRYDAEYYIENQVLPAV
ERILKAFGYRKEDLRYQKTKQVGLGAWLKVKGKKLEHHHHHH*
Stoffel fragment (SEQ ID NO: 77)
Stoffel fragment of the DNA polymerase from Thermus aquaticus Extinction coefficient: 70,360 M⁻¹
cm⁻¹ Mass: 62.0 kDa
MALEEAPWPPPEGAFVGFVLSRKEPMWADLLALAAARGGRVHRAPEPYKALRDLKEARGLLAKDL
SVLALREGLGLPPGDDPMLLAYLLDPSNTTPEGVARRYGGEWTEEAGERAALSERLFANLWGRLE
GEERLLWLYREVERPLSAVLAHMEATGVRLDVAYLRALSLEVAEEIARLEAEVFRLAGHPFNLNSR
DQLERVLFDELGLPAIGKTEKTGKRSTSAAVLEALREAHPIVEKILQYRELTKLKSTYIDPLPDLIHPR
TGRLHTRFNQTATATGRLSSSDPNLQNIPVRTPLGQRIRRAFIAEEGWLLVALDYSQIELRVLAHLS
GDENLIRVFQEGRDIHTETASWMFGVPREAVDPLMRRAAKTINFGVLYGMSAHRLSQELAIPYEEA
QAFIERYFQSFPKVRAWIEKTLEEGRRRGYVETLFGRRRYVPDLEARVKSVREAAERMAFNMPVQ
GTAADLMKLAMVKLFPRLEEMGARMLLQVHDELVLEAPKERAEAVARLAKEVMEGVYPLAVPLEV
EVGIGEDWLSAKELEHHHHHH*
SFM4-6 (SEQ ID NO: 78)
Stoffel fragment of the DNA polymerase from Thermus aquaticus with I322E E323G D363N L366M
E389K E450N M455R mutations Extinction coefficient: 70,360 M⁻¹cm⁻¹ Mass:62.0 kDa
MALEEAPWPPPEGAFVGFVLSRKEPMWADLLALAAARGGRVHRAPEPYKALRDLKEARGLLAKDL
SVLALREGLGLPPGDDPMLLAYLLDPSNTTPEGVARRYGGEWTEEAGERAALSERLFANLWGRLE
GEERLLWLYREVERPLSAVLAHMEATGVRLDVAYLRALSLEVAEEIARLEAEVFRLAGHPFNLNSR
DQLERVLFDELGLPAIGKTEKTGKRSTSAAVLEALREAHPIVEKILQYRELTKLKSTYIDPLPDLIHPR
TGRLHTRFNQTATATGRLSSSDPNLQNIPVRTPLGQRIRRAFIAEEGWLLVALDYSQEGLRVLAHLS
GDENLIRVFQEGRDIHTETASWMFGVPREAVNPMMRRAAKTINFGVLYGMSAHRLSQKLAIPYEEA
QAFIERYFQSFPKVRAWIEKTLEEGRRRGYVETLFGRRRYVPDLEARVKSVRNAAERRAFNMPVQ
GTAADLMKLAMVKLFPRLEEMGARMLLQVHDELVLEAPKERAEAVARLAKEVMEGVYPLAVPLEV
EVGIGEDWLSAKELEHHHHHH*
TfPol (SEQ ID NO: 79)
Stoffel fragment homolog from Thermus filiformis (wild-type) Extinction coefficient: 56,380
M⁻¹cm⁻¹ Mass: 62.0 kDa
MPREEAPWPPPEGAFVGFLLSRKEPMWAELLALAAAAEGRVHRATSPVEALADLKEARGFLAKDL
AVLALREGVALDPTDDPLLVAYLLDPANTNPEGVARRYGGEFTEDAAERALLSERLFQNLFPRLSE
KLLWLYQEVERPLSRVLAHMEARGVRLDVPLLEALSFELEKEMERLEGEVFRLAGHPFNLNSRDQL
ERVLFDELGLTPVGRTEKTGKRSTAQGALEALRGAHPIVELILQYRELSKLKSTYLDPLPRLVHPRT
GRLHTRFNQTATATGRLSSSDPNLQNIPVRTPLGQRIRKAFVAEEGWLLLAADYSQIELRVLAHLSG
DENLKRVFREGKDIHTETAAWMFGLDPALVDPKMRRAAKTVNFGVLYGMSAHRLSQELGIDYKEA
EAFIERYFQSFPKVRAWIERTLEEGRTRGYVETLFGRRRYVPDLASRVRSVREAAERMAFNMPVQ
GTAADLMKIAMVKLFPRLKPLGAHLLLQVHDELVLEVPEDRAEEAKALVKEVMENTYPLDVPLEVEV
GVGRDWLEAKGDLEHHHHHH*
TfPol* (SEQ ID NO: 80)
Stoffel fragment homolog from Thermus filiformis containing point mutations I341E E342G D382N
L384M E408K E469N M474R shown to enhance the substrate promiscuity of SF4-6 Extinction
coefficient: 56,380 M⁻¹cm⁻¹ Mass: 63.0 kDa
MGSSHHHHHHSSGLVPRGSHMPREEAPWPPPEGAFVGFLLSRKEPMWAELLALAAAAEGRVHR
ATSPVEALADLKEARGFLAKDLAVLALREGVALDPTDDPLLVAYLLDPANTNPEGVARRYGGEFTE
DAAERALLSERLFQNLFPRLSEKLLWLYQEVERPLSRVLAHMEARGVRLDVPLLEALSFELEKEME
RLEGEVFRLAGHPFNLNSRDQLERVLFDELGLTPVGRTEKTGKRSTAQGALEALRGAHPIVELILQY
RELSKLKSTYLDPLPRLVHPRTGRLHTRFNQTATATGRLSSSDPNLQNIPVRTPLGQRIRKAFVAEE
GWLLLAADYSQEGLRVLAHLSGDENLKRVFREGKDIHTETAAWMFGLDPALVNPMMRRAAKTVNF
GVLYGMSAHRLSQVLGIDYKEAEAFIERYFQSFPKVRAWIERTLEEGRTRGYVETLFGRRRYVPDL
ASRVRSVRNAAERRAFNMPVQGTAADLMKIAMVKLFPRLKPLGAHLLLQVHDELVLEVPEDRAEEA
KALVKEVMENTYPLDVPLEVEVGVGRDWLEAKGD*
MhPol* (SEQ ID NO: 81)
Stoffel fragment homolog from Marinithermus hydrothermalis containing point mutations I144Y
I343E E344G D384N L386M E410K E471N M476R shown to enhance the substrate promiscuity of
SF4-6 Extinction coefficient: 90,300 M⁻¹cm⁻¹ Mass: 63.0 kDa
MGSSHHHHHHSSGLVPRGSHMAEEAPWPPPPEAFLGYVLDRPQPMWAELKGLAGAWEGRVAR
GPARAKELARFEAVHALQAKDLTVWARREGVRVQPGEDPLLLAYLYDPTNSDPAATVRRYGAGD
WSEDPAARALAAAELWRYLGERLAGEEALWWLYREVERPLAGVLAEMEHAGVRVDVAYLEALSA
ELGREIAAIEAEVHRLAGRAFNLNSRDQLEVILYDELGLTPTRRTQKTGRRSTSAAALEALVGAHPIV
ERILAYRELSKLKGTYLDPLPRLVHPATGRIHTRYHQTGTATGRLSSSDPNLQNIPVRTEVGRRIRR
AFVAEPGYVLVAADYSQEGLRVLAHLSGDENLKRVFQERRDIHTQTASWMFGVPPEAVNPMRRR
AAKTVNFGVLYGMSPHRLSRVLGIEYAEAERFIQRYFESYPRVQAYIERTLEQAREKGYVETLFGR
RRYIPDIRSRNRNVRNAAERRAFNMPVQGTAADLMKLAMVKLAPEIRSLGARLILQVHDELVLEAPQ
ERAEAVARVVREVMEGAWALDVPLEVEVGIGENWLEAK*
MhPol (SEQ ID NO: 82)
Stoffel fragment homolog from Marinithermus hydrothermalis Extinction coefficient: 90,300 M⁻¹cm⁻¹
Mass: 61.8 kDa
MAEEAPWPPPPEAFLGYVLDRPQPMWAELKGLAGAWEGRVARGPARAKELARFEAVHALQAKDL
TVWARREGVRVQPGEDPLLLAYLYDPTNSDPAATVRRYGAGDWSEDPAARALAAAELWRYLGER
LAGEEALWWLYREVERPLAGVLAEMEHAGVRVDVAYLEALSAELGREIAAIEAEVHRLAGRAFNLN
SRDQLEVILYDELGLTPTRRTQKTGRRSTSAAALEALVGAHPIVERILAYRELSKLKGTYLDPLPRLV
HPATGRIHTRYHQTGTATGRLSSSDPNLQNIPVRTEVGRRIRRAFVAEPGYVLVAADYSQEGLRVL
AHLSGDENLKRVFQERRDIHTQTASWMFGVPPEAVNPMRRRAAKTVNFGVLYGMSPHRLSRVLGI
EYAEAERFIQRYFESYPRVQAYIERTLEQAREKGYVETLFGRRRYIPDIRSRNRNVRDAAERMAFN
MPVQGTAADLMKLAMVKLAPEIRSLGARLILQVHDELVLEAPQERAEAVARVVREVMEGAWALDV
PLEVEVGIGENWLEAKLEHHHHHH*
KOD DGLNK KOD variant N210D, Y409G, A485L, D614N, E664K (Ref. 21) (SEQ ID NO: 83)
MILDTDYITEDGKPVIRIFKKENGEFKIEYDRTFEPYFYALLKDDSAIEEVKKITAERHGTVVTVKRVE
KVQKKFLGRPVEVWKLYFTHPQDVPAIRDKIREHPAVIDIYEYDIPFAKRYLIDKGLVPMEGDEELKM
LAFDIETLYEEGEEFAEGPILMISYADEEGARVITWKNVDLPYVDVVSTEREMIKRFLRVVKEKDPDV
LITYDGDNFDFAYLKKRCEKLGINFALGRDGSEPKIQRMGDRFAVEVKGRIHFDLYPVIRRTINLPTY
TLEAVYEAVFGQPKEKVYAEEITTAWETGENLERVARYSMEDAKVTYELGKEFLPMEAQLSRLIGQ
SLWDVSRSSTGNLVEWFLLRKAYERNELAPNKPDEKELARRRQSYEGGYVKEPERGLWENIVYLD
FRSLGPSIIITHNVSPDTLNREGCKEYDVAPQVGHRFCKDFPGFIPSLLGDLLEERQKIKKKMKATID
PIERKLLDYRQRLIKILANSYYGYYGYARARWYCKECAESVTAWGREYITMTIKEIEEKYGFKVIYSD
TDGFFATIPGADAETVKKKAMEFLKYINAKLPGALELEYEGFYERGFFVTKKKYAVIDEEGKITTRGL
EIVRRNWSEIAKETQARVLEALLKDGDVEKAVRIVKEVTEKLSKYEVPPEKLVIHKQITRDLKDYKAT
GPHVAVAKRLAARGVKIRPGTVISYIVLKGSGRIGDRAIPFDEFDPTKHKYDAEYYIENQVLPAVERIL
RAFGYRKEDLRYQKTRQVGLSAWLKPKGTLEHHHHHH

Endonuclease V Sequences (Table 2)

| | EndonucleaseV | Protein Sequence |
|---|---|---|
| SEQ ID | TmEndo | MDYRQLHRWDLPPEEAIKVQNELRKKIKLTPYEGEPEYVAGVDLSFPGKEE |
| NO: 84 | from | GLAVIVVLEYPS |
| | Thermotoga | FKILEVVSERGEITFPYIPGLLAFREGPLFLKAWEKLRTKPDVVVFNGQGLAH |
| | maritima | PRKLGIASHMG |
| | 26,662 Da | LFIEIPTIGVAKSRLYGTFKMPEDKRCSWSYLYDGEEIIGCVIRTKEGSAPIFV |
| | 29910 M⁻¹cm⁻¹ | SPGHLMDVESS |
| | | KRLIKAFTLPGRRIPEPTRLAHIYTQRLKKGLFLEHHHHHH |
| SEQ ID | TnEndoV | MHMTYKKLHEWDLSPEEAMKIQNVLREKILFKPFEGEPKYVAGVDLSFPKRE |
| NO: 85 | from | EGLAVIVVM |
| | Thermotoga | EYPTFKIVELVSERGKVDFPYIPGLLAFREGPLFLKAWEKLKTKPDVVVFDGQ |
| | neapolitana | GIAHPRKLGIA |
| | 26,999 Da | SHMGLFIEIPTIGVAKSRLYGTYREPENRRCSWSYLYDNEEIIGCVMRTREG |
| | 29910 M⁻¹cm⁻¹ | SAPIFVSPGHLI |
| | | DVESSIRLVKSFTLPGRRLPEPTRMAHIYTQRLKKGLLEHHHHHH |
| SEQ ID | PfEndoV | MHMKYEKLHEWNVSVEEAIKIQNQLRSKIKLMPLKEEKIRYVAGVDLSFPEP |
| NO: 86 | from | NLGYAVIVVID |
| | Pseudo- | LFNMSIVENVAAKEKVVFDYIPGLLAFREGPVFLKAWENLKTKPNLVVFDGH |
| | thermotoga | GIAHPRGLGI |
| | thermarum | ASHMGLWINLPTIGVAKSKLYGFYKGLQQAKWSEVPLLDPSGNQIGIVIRTKE |
| | 26,835 Da | NADPIFVSP |
| | 30940 M⁻¹cm⁻¹ | GHLCDLDSASKIVKKMCLENNRLPEPTRLAHLLTQKIHRRLEHHHHHH |
| SEQ ID | TmeEndoV | MHMEYKKLHSWKLIPEKAIELQKLLSKKLTFKPLSKEINLVAGVDLSFVKDYG |
| NO: 87 | from | LAVIVILDKNM |
| | Thermosipho | ELIKVYHHIEKITFPYIPGLLAFREGPIFLKAWKKVNHNVDVVFFDGHGISHPR |
| | melanesiensis | SMGIASHMG |
| | 27526 Da | LWIEQPTIGIAKKILFGNYVEPENKKFSFTYITYKNQKIGIVLRSRENVKPIFISP |
| | 29910 M⁻¹cm⁻¹ | GNLITLDESLEL |
| | | TKQFITKYKLPEPTRLAHKYSQLLKKQFNEELQLEHHHHHH |
| SEQ ID | TaEndoV | MHMLYKKLHDWNISPKKAIDIQSTLKKLLKFEKIEKEISLVAGVDLSFFKEYGL |
| NO: 88 | from | AVIVVLDYDF |
| | Thermosipho | NIREIVHHVEKVSYPYIPGLLAFREGPIFIKAWEKLKTDVDIVFFDGHGIAHPR |
| | atlanticus | KLGIASHMGL |
| | 27,172 Da | WIEKATIGIAKSKLIGNYKEPGKKRGNFSYLEYDGEKIGIVYRSRNNVKPIFISP |
| | 34380 M⁻¹cm⁻¹ | GYLIDLESSLKI |
| | | TKSFITKYRLPEPTRLAHLYTQKIKKEHLSLEHHHHHH |

TABLE 4ii
Reaction conditions for the synthesis of P19-P32. All reactions were performed
using template (4 μM), TnEndoV (2 μM) and were incubated for 12 hours at 70° C.

| | | | | | Formamide | |
|---|---|---|---|---|---|---|
| Product | [dNTP] (mM) | Pol. | [Pol.] (μM) | Buffer | (%) | Template |
| P19 | 4 | KOD | 2 | A | 10 | T22 |
| P20 | 1 | KOD | 2 | B | 5 | T22 |
| P21 | 1 | TfPol* | 1 | C | 10 | T22 |
| P22 | 1 | KOD | 2 | D | 5 | T23 |
| | | DGLNK | | | | |
| P23 | 1 | KOD | 2 | D | 5 | T24 |
| | | DGLNK | | | | |
| P24 | 2.5 | KOD | 2 | B | 5 | T22 |
| P25 | 2.5 | KOD | 2 | B | 5 | T22 |
| P26 | 2.5 | TfPol* | 1 | C | 10 | T22 |
| P27 | 2.5 | TfPol* | 1 | C | 10 | T22 |
| P28 | 2.5 | TfPol* | 1 | C | 10 | T22 |
| P29 | 1 | KOD | 1 | D | 5 | T22 |
| P30 | 1 | TfPol* | 1 | C | 10 | T22 |
| P31 | 1 | TfPol* | 1 | C | 10 | T22 |
| P32 | 1 | TfPol* | 1 | C | 10 | T22 |

†dNTP mix contained 1 mM 2′F-ATP and 0.5 mM of each of the other NTPs
Buffer A: 20 mM Tris-HCl (pH 8), 20 mM MgCl₂, 100 mM potassium glutamate, 100 mM arginine HCl, 0.01 mg/ml acetylated BSA, 10 mM DTT, 0.2M trehalose and 1M 1,2-propanediol
Buffer B: 50 mM Tris-AcOH (pH 8), 20 mM MgSO₄, 50 mM KOAc, 100 mM potassium glutamate, 100 mM arginine HCl, 0.01 mg/ml acetylated BSA, 0.1% Triton X-100 ® and 30 mM DTT
Buffer C contains 20 mM Tris-HCl (pH 8.8 @ 25° C.), 10 mM (NH4)2SO4, 10 mM KCl, 2 mM MgSO4, 0.1% Triton ® X-100 and 2 mM NiCl₂.
Buffer D: 20 mM Tris-HCl (pH 8), 20 mM MgCl₂, 100 mM potassium glutamate, 100 mM arginine HCl, 0.01 mg/ml acetylated BSA, 10 mM DTT

1. A method for producing a single stranded oligonucleotide, the method comprising:
i. providing a primer-template comprising a) a primer for initiation of oligonucleotide synthesis, b) a template which directs synthesis of an oligonucleotide product, and c) a cleavable site to enable release of the oligonucleotide product from the template;
ii. incubating the primer-template with a nucleic acid polymerase, one or more dNTPs, and a cleaving agent to form a reaction mixture;
iii. maintaining the reaction mixture under conditions to allow for extension of the primer by the polymerase to form an extended primer-template, and cleavage of the extended primer-template at the cleavable site by the cleaving agent to provide an oligonucleotide product;
iv. optionally separating the oligonucleotide product from the reaction mixture.

2. A method according to claim 1 wherein the polymerase is polymerase from Thermococcus kodakaraensis (KOD), Stoffel, family B polymerase 9° N, Thermus filiformis (TfPol) and Marinithermus hydrothermalis (MhPol); Klenow fragment from E.coli, T4 and T7 polymerase, SFP1, Stoffel variant SFM4-6, Stoffel homologs from Thermus filiformis and Marinithermus hydrothermalis (Mhpol) as shown in Table 3, and variants of any of the afore-mentioned polymerases as shown in Table 3 having the listed mutations.

3. A method of producing population of single stranded oligonucleotides, the method comprising:
i) providing a primer-template comprising a) a primer for initiation of oligonucleotide synthesis, b) a template which directs synthesis of an oligonucleotide product, and c) a cleavable site to enable release of the oligonucleotide product from the template;
ii) incubating the primer-template with a nucleic acid polymerase, one or more dNTPs, and a cleaving agent to form a reaction mixture;
iii) maintaining the reaction mixture under conditions to allow for extension of the primer by the polymerase to form an extended primer-template, and cleavage of the extended primer-template at the cleavable site by the cleaving agent to provide an oligonucleotide product;
iv) optionally separating the oligonucleotide product from the reaction mixture.
wherein the nucleotides include modified thiotriphosphates (NTPaS) and wherein the oligonucleotide population comprises substantially the same stereoisomer;
and wherein the polymerase is selected from the group consisting of polymerase from Thermococcus kodakaraensis (KOD), Stoffel, family B polymerase 9° N, Thermus filiformis (TfPol) and Marinithermus hydrothermalis (MhPol); Klenow fragment from E.coli, T4 and T7 polymerase, SFP1, Stoffel variant SF4-6, Stoffel homologs from Thermus filiformis and Marinithermus hydrothermalis (Mhpol) as shown in Table 3, and variants of any of the afore-mentioned polymerases as shown in Table 3.

4. A method according to any one of claims 1 to 3, wherein the reagents substantially do not comprise acetonitrile.

5. A method according to any one of claims 1 to 4 wherein the cleaving is mediated by an endonuclease V, preferably TmEndoV from Thermotoga maritima (TmEndoV). TmEndoV from Thermotoga neapolitana, PtEndov from Pseudothermotoga thermarum, TmeEndoV from Thermosipho melanesiensis, and TaEndoV from Thermosipho atlanticus; or a variant thereof.

6. A method according to any one of claims 1 to 5 wherein the step of maintaining the reaction mixture in step iii) comprises maintaining the reaction mixture for a reaction period, wherein a reaction period is a duration which allows time for two or more cycles of extension and cleavage, within the same reaction mixture.

7. A method according to any one of claims 1 to 6 wherein the reaction conditions of step iii) comprise a reaction temperature at or above the melting temperature of the template and extension product, and preferably the reaction conditions of step iii) are isothermal.

8. A method according to any one of claims 1 to 7 wherein the method further comprises one or more steps of: dissociation of an oligonucleotide product from the template; isolation of the oligonucleotide product from a reaction mixture; and purification of the oligonucleotide product.

9. A kit for producing a single stranded oligonucleotide, the kit comprising:
i. a primer-template comprising a) a primer for initiation of oligonucleotide synthesis, b) a template which directs synthesis of an oligonucleotide product, and c) a cleavable site to enable release of the oligonucleotide product from the template; and optionally one or more of:
ii) a nucleic acid polymerase and optionally nucleotides
iii) a cleaving agent.

10. A kit according to claim 9 comprising a first container containing a primer-template, and a second or further container contain nucleotides, a polymerase, and/or a buffer.

11. A reaction mixture for producing a single stranded oligonucleotide, the reaction mixture comprising a primer-template which comprises a) a primer for initiation of oligonucleotide synthesis. b) a template which directs synthesis of an oligonucleotide product, and c) a cleavable site to enable release of the oligonucleotide product from the template; and:
i. a nucleic acid polymerase and optionally nucleotides
ii. a cleaving agent
iii. an oligonucleotide product, which is complementary to the template sequence.

12. A method, kit, reaction, or support according to as defined in any one of claims 1 to 11 wherein the cleavage site is a deaminated base, preferably inosine.

13. A method, kit, reaction, or support according to as defined in any one of claims 1 to 12 wherein the template sequence is complement of a therapeutic RNA.

14. A method, kit, reaction, or support according to any one of claims 1 to 13 wherein the oligonucleotide product is a therapeutic RNA, preferably an miRNA or antisense oligonucleotide (ASO), or an aptamer.

15. A method, kit, reaction, or support according to any one of claims 1 to 14 wherein the oligonucleotide product comprise one or more modified nucleotides.

16. A method, kit, reaction, or support according to any one of claims 1 to 15 wherein the polymerase is as defined in Table 3 or a variant thereof; the cleavage system comprises an endonuclease V, and the cleavable site is an inosine residue.

17. A method, reaction, or kit according to any one of claims 1 to 16 wherein the nucleotides comprise natural and/or modified nucleotides, and wherein modified include nucleotides comprising modified bases, sugars, or internucleosidic linkages, for example phosphorothioate internucleosidic linkages, 5′-N-phosphoramidite linkages, bases containing linking groups permitting the attachment of labels, such as fluorophores, or haptens.

18. A method, kit, reaction, or support according to any one of claims 1 to 17 wherein the nucleic acid molecule comprises an immobilisation moiety, preferably wherein the immobilisation moiety is biotin.

19. A support for producing a single stranded oligonucleotide, comprising immobilised thereon a population of primer-templates, wherein each nucleic acid molecule comprises a) a primer for initiation of oligonucleotide synthesis, b) a template which directs synthesis of an oligonucleotide product, and c) a cleavable site to enable release of the oligonucleotide product from the template.

20. A support according to claim 19, wherein the support is an array, preferably wherein the array comprises 100, 1000, 10,000, 100,000, 1,000,000, 10,000,000, 100,000,000, 1,000,000,000 or more nucleic acid molecules immobilized thereon.

21. A population of oligonucleotides produced by a method of any one of claims 1 to 20.

23. A population of oligonucleotides according to claim 21, wherein the population is at least 70% pure.

24. A cell for producing a single stranded oligonucleotide, comprising a primer-template which comprises a) a primer for initiation of oligonucleotide synthesis, b) a template which directs synthesis of an oligonucleotide product, and c) a cleavable site to enable release of the oligonucleotide product from the template.


SEQUENCE LISTING (105 sequences)

SEQ ID NO: 1 - DNA, 41 bp, synthetic construct
  source 1..41: mol_type = other DNA
  misc_feature 1..41: note = T1
  modified_base 40: mod_base = OTHER; note = deoxyinosine
ccagcattatgaaagtgacacgtgcaccattggtgcacgng

SEQ ID NO: 2 - DNA, 59 bp, synthetic construct
  source 1..59: mol_type = other DNA
  misc_feature 1..59: note = E1
  modified_base 40: mod_base = OTHER; note = deoxyinosine
ccagcattatgaaagtgacacgtgcaccattggtgcacgngtcactttcataatgctgg

SEQ ID NO: 3 - DNA, 18 bp, synthetic construct
  source 1..18: mol_type = other DNA
  misc_feature 1..18: note = P1
  misc_feature 1: note = 5'-phosphate
tcactttcataatgctgg

SEQ ID NO: 4 - DNA, 41 bp, synthetic construct; identical to SEQ ID NO: 1
  source 1..41: mol_type = other DNA
  misc_feature 1..41: note = T2
  misc_feature 1: note = 5' biotin
  modified_base 40: mod_base = OTHER; note = deoxyinosine

SEQ ID NO: 5 - DNA, 41 bp, synthetic construct; identical to SEQ ID NO: 1
  source 1..41: mol_type = other DNA
  misc_feature 1..41: note = T3
  modified_base 30: mod_base = OTHER; note = biotin-modified thymidine residue
  modified_base 40: mod_base = OTHER; note = deoxyinosine

SEQ ID NO: 6 - DNA, 40 bp, synthetic construct
  source 1..40: mol_type = other DNA
  misc_feature 1..40: note = T4
  modified_base 39: mod_base = OTHER; note = deoxyinosine
ccagcattatgaaagtgacacgtgcaccatggtgcacgng

SEQ ID NO: 7 - DNA, 41 bp, synthetic construct
  source 1..41: mol_type = other DNA
  misc_feature 1..41: note = T5
  modified_base 40: mod_base = OTHER; note = deoxyinosine
ccagcattatgaaagtgacacgggcgccgtcggcgcccgng

SEQ ID NO: 8 - DNA, 41 bp, synthetic construct
  source 1..41: mol_type = other DNA
  modified_base 40: mod_base = OTHER; note = deoxyinosine
  misc_feature 1..41: note = T6
ccagcattatgaaagtgactcgtgcaccattggtgcacgng

SEQ ID NO: 9 - DNA, 41 bp, synthetic construct
  source 1..41: mol_type = other DNA
  misc_feature 1..41: note = T7
  modified_base 40: mod_base = OTHER; note = deoxyinosine
ccagcattatgaaagtgacccgtgcaccattggtgcacgng

SEQ ID NO: 10 - DNA, 37 bp, synthetic construct
  source 1..37: mol_type = other DNA
  misc_feature 1..37: note = T8
  modified_base 36: mod_base = OTHER; note = deoxyinosine
ccagcattatgaaagtgacacgtgcaatttgcacgng

SEQ ID NO: 11 - DNA, 45 bp, synthetic construct
  source 1..45: mol_type = other DNA
  misc_feature 1..45: note = T9
  modified_base 44: mod_base = OTHER; note = deoxyinosine
ccagcattatgaaagtgacacgtgcacgccattggcgtgcacgng

SEQ ID NO: 12 - DNA, 72 bp, synthetic construct
  source 1..72: mol_type = other DNA
  misc_feature 1..72: note = FRET template
  modified_base 9: mod_base = OTHER; note = i5-isomer of TAMRA
  modified_base 34: mod_base = OTHER; note = fluorescein is attached to position 5 of the thymine ring by a 6-carbon spacer arm
cgtcgacgtaggaggaaaggacatcttctagcatacgtcgacgatgcatgcactggtcagaattcgcaccac

SEQ ID NO: 13 - DNA, 17 bp, synthetic construct
  source 1..17: mol_type = other DNA
  misc_feature 1..17: note = FRET primer
gtggtgcgaattctgac

SEQ ID NO: 14 - DNA, 30 bp, synthetic construct
  source 1..30: mol_type = other DNA
  misc_feature 1..30: note = T10
  modified_base 29: mod_base = OTHER; note = deoxyinosine
agctagccacgtgcaccaatggtgcacgng

SEQ ID NO: 15 - DNA, 30 bp, synthetic construct
  source 1..30: mol_type = other DNA
  misc_feature 1..30: note = T11
  modified_base 29: mod_base = OTHER; note = deoxyinosine
ctagctacacgtgcaccaatggtgcacgng

SEQ ID NO: 16 - DNA, 30 bp, synthetic construct
  source 1..30: mol_type = other DNA
  misc_feature 1..30: note = T12
  modified_base 29: mod_base = OTHER; note = deoxyinosine
gctagctcacgtgcaccaatggtgcacgng

SEQ ID NO: 17 - DNA, 29 bp, synthetic construct
  source 1..29: mol_type = other DNA
  misc_feature 1..29: note = T13
  modified_base 28: mod_base = OTHER; note = deoxyinosine
tgacgacacgtgcaccaatggtgcacgng

SEQ ID NO: 18-21 - skipped (4 entries)

SEQ ID NO: 22 - DNA, 59 bp, synthetic construct; identical to SEQ ID NO: 2
  misc_feature 1..41: note = DNA
  misc_feature 42..59: note = RNA
  source 1..59: mol_type = other DNA
  modified_base 40: mod_base = OTHER; note = deoxyinosine
  modified_base 42..59: mod_base = OTHER; note = 2'-fluoro
  misc_feature 1..59: note = E2

SEQ ID NO: 23 - RNA, 18 nt, synthetic construct
  source 1..18: mol_type = other RNA
  misc_feature 1..18: note = P6
  modified_base 1..18: mod_base = OTHER; note = 2'-fluoro
  misc_feature 1: note = 5'-phosphate
tcactttcataatgctgg

SEQ ID NO: 24 - DNA, 59 bp, synthetic construct; identical to SEQ ID NO: 2
  misc_feature 1..41: note = DNA
  misc_feature 42..59: note = RNA
  source 1..59: mol_type = other DNA
  misc_feature 1..59: note = E3
  modified_base 40: mod_base = OTHER; note = deoxyinosine
  modified_base 42: mod_base = um
  modified_base 43: mod_base = cm
  modified_base 44: mod_base = OTHER; note = 2'-OMe
  modified_base 45: mod_base = cm
  modified_base 46..48: mod_base = um
  modified_base 49: mod_base = cm
  modified_base 50: mod_base = OTHER; note = 2'-OMe
  modified_base 51: mod_base = um
  modified_base 52..53: mod_base = OTHER; note = 2'-OMe
  modified_base 54: mod_base = um
  modified_base 55: mod_base = gm
  modified_base 56: mod_base = cm
  modified_base 57: mod_base = um
  modified_base 58..59: mod_base = gm

SEQ ID NO: 25 - RNA, 18 nt, synthetic construct
  source 1..18: mol_type = other RNA
  misc_feature 1..18: note = P7
  misc_feature 1: note = 5'-phosphate
  modified_base 1: mod_base = um
  modified_base 2: mod_base = cm
  modified_base 3: mod_base = OTHER; note = 2'-OMe
  modified_base 4: mod_base = cm
  modified_base 5..7: mod_base = um
  modified_base 8: mod_base = cm
  modified_base 9: mod_base = OTHER; note = 2'-OMe
  modified_base 10: mod_base = um
  modified_base 11..12: mod_base = OTHER; note = 2'-OMe
  modified_base 13: mod_base = um
  modified_base 14: mod_base = gm
  modified_base 15: mod_base = cm
  modified_base 16: mod_base = um
  modified_base 17..18: mod_base = gm
tcactttcataatgctgg

SEQ ID NO: 26 - DNA, 59 bp, synthetic construct; identical to SEQ ID NO: 2
  source 1..59: mol_type = other DNA
  misc_feature 1..59: note = E4
  modified_base 40: mod_base = OTHER; note = deoxyinosine
  modified_base 42..59: mod_base = OTHER; note = 2'-methoxyethoxy
  misc_feature 1..41: note = DNA
  misc_feature 42..59: note = RNA

SEQ ID NO: 27 - DNA, 18 bp, synthetic construct
  source 1..18: mol_type = other DNA
  misc_feature 1..18: note = P8
  modified_base 1..18: mod_base = OTHER; note = 2'-methoxyethoxy
  misc_feature 1: note = 5'-phosphate
tcactttcataatgctgg

SEQ ID NO: 28 - DNA, 58 bp, synthetic construct
  source 1..58: mol_type = other DNA
  modified_base 41: mod_base = OTHER; note = LNA
  modified_base 39: mod_base = OTHER; note = deoxyinosine
  misc_feature 1..58: note = E5
  misc_feature 1..40: note = DNA
  misc_feature 41: note = RNA
  misc_feature 42..58: note = DNA
cagcattatgaaagtgacacgtgcaccattggtgcacgngtcactttcataatgctgg

SEQ ID NO: 29 - DNA, 18 bp, synthetic construct
  source 1..18: mol_type = other DNA
  modified_base 1: mod_base = OTHER; note = LNA
  misc_feature 2..18: note = DNA
  misc_feature 1..18: note = P9
  misc_feature 1: note = 5'-phosphate
  misc_feature 1: note = RNA
tcactttcataatgctgg

SEQ ID NO: 30 - DNA, 40 bp, synthetic construct
  source 1..40: mol_type = other DNA
  misc_feature 1..40: note = T14
  modified_base 39: mod_base = OTHER; note = deoxyinosine
cagcattatgaaagtgacacgtgcaccattggtgcacgng

SEQ ID NO: 31 - DNA, 59 bp, synthetic construct; identical to SEQ ID NO: 2
  source 1..59: mol_type = other DNA
  misc_feature 1..59: note = E6
  modified_base 40: mod_base = OTHER; note = deoxyinosine
  modified_base 41..59: mod_base = OTHER; note = 5'-phosphorothioate linkage

SEQ ID NO: 32 - DNA, 18 bp, synthetic construct
  source 1..18: mol_type = other DNA
  misc_feature 1..18: note = P10
  modified_base 1..18: mod_base = OTHER; note = 5'-phosphorothioate linkage
tcactttcataatgctgg

SEQ ID NO: 33 - DNA, 59 bp, synthetic construct; identical to SEQ ID NO: 2
  source 1..59: mol_type = other DNA
  misc_feature 1..59: note = E7
  modified_base 40: mod_base = OTHER; note = deoxyinosine
  modified_base 41..42: mod_base = OTHER; note = 5'-phosphorothioate linkage

SEQ ID NO: 34 - DNA, 59 bp, synthetic construct
  source 1..59: mol_type = other DNA
  misc_feature 1..59: note = E8
  modified_base 40: mod_base = OTHER; note = deoxyinosine
  modified_base 41..42: mod_base = OTHER; note = 5'-phosphorothioate linkage
ccagcattatgaaagtgtcacgtgcaccattggtgcacgngacactttcataatgctgg

SEQ ID NO: 35 - DNA, 59 bp, synthetic construct
  source 1..59: mol_type = other DNA
  misc_feature 1..59: note = E9
  modified_base 40: mod_base = OTHER; note = deoxyinosine
  modified_base 41..42: mod_base = OTHER; note = 5'-phosphorothioate linkage
ccagcattatgaaagtggcacgtgcaccattggtgcacgngccactttcataatgctgg

SEQ ID NO: 36 - DNA, 59 bp, synthetic construct
  source 1..59: mol_type = other DNA
  misc_feature 1..59: note = E10
  modified_base 40: mod_base = OTHER; note = deoxyinosine
  modified_base 41..42: mod_base = OTHER; note = 5'-phosphorothioate linkage
ccagcattatgaaagtgccacgtgcaccattggtgcacgnggcactttcataatgctgg

SEQ ID NO: 37 - DNA, 59 bp, synthetic construct; identical to SEQ ID NO: 2
  source 1..59: mol_type = other DNA
  misc_feature 1..59: note = E11
  modified_base 40: mod_base = OTHER; note = deoxyinosine
  modified_base 41..42: mod_base = OTHER; note = 5'-Rp phosphorothioate linkage
  modified_base 45..48: mod_base = OTHER; note = 5'-Rp phosphorothioate linkage
  modified_base 50..51: mod_base = OTHER; note = 5'-Rp phosphorothioate linkage
  modified_base 53..54: mod_base = OTHER; note = 5'-Rp phosphorothioate linkage
  modified_base 56..57: mod_base = OTHER; note = 5'-Rp phosphorothioate linkage

SEQ ID NO: 38 - DNA, 59 bp, synthetic construct
  source 1..59: mol_type = other DNA
  misc_feature 1..59: note = E12
  modified_base 40: mod_base = OTHER; note = deoxyinosine
  modified_base 41..42: mod_base = OTHER; note = 5'-Rp phosphorothioate linkage
  modified_base 43..44: mod_base = OTHER; note = 5'-Rp phosphorothioate linkage
  modified_base 49..50: mod_base = OTHER; note = 5'-Rp phosphorothioate linkage
  modified_base 51..53: mod_base = OTHER; note = 5'-Rp phosphorothioate linkage
ccagcattatgaaagtgacacgtgcaccattggtgcacgngacactttcataatgctgg

SEQ ID NO: 39 - DNA, 59 bp, synthetic construct
  source 1..59: mol_type = other DNA
  misc_feature 1..59: note = E13
  modified_base 40: mod_base = OTHER; note = deoxyinosine
  modified_base 41..43: mod_base = OTHER; note = 5'-Rp phosphorothioate linkage
  modified_base 44..45: mod_base = OTHER; note = 5'-Rp phosphorothioate linkage
  modified_base 48..49: mod_base = OTHER; note = 5'-Rp phosphorothioate linkage
  modified_base 55..56: mod_base = OTHER; note = 5'-Rp phosphorothioate linkage
ccagcattatgaaagtgacacgtgcaccattggtgcacgngccactttcataatgctgg

SEQ ID NO: 40 - DNA, 59 bp, synthetic construct
  source 1..59: mol_type = other DNA
  misc_feature 1..59: note = E14
  modified_base 40: mod_base = OTHER; note = deoxyinosine
  modified_base 41..42: mod_base = OTHER; note = 5'-Rp phosphorothioate linkage
  modified_base 54..55: mod_base = OTHER; note = 5'-Rp phosphorothioate linkage
  modified_base 57..59: mod_base = OTHER; note = 5'-Rp phosphorothioate linkage
ccagcattatgaaagtgacacgtgcaccattggtgcacgnggcactttcataatgctgg

SEQ ID NO: 41 - DNA, 41 bp, synthetic construct
  source 1..41: mol_type = other DNA
  misc_feature 1..41: note = T15
  modified_base 40: mod_base = OTHER; note = deoxyinosine
ccagcattatgaaagtgtcacgtgcaccattggtgcacgng

SEQ ID NO: 42 - DNA, 41 bp, synthetic construct
  source 1..41: mol_type = other DNA
  misc_feature 1..41: note = T16
  modified_base 40: mod_base = OTHER; note = deoxyinosine
ccagcattatgaaagtggcacgtgcaccattggtgcacgng

SEQ ID NO: 43 - DNA, 41 bp, synthetic construct
  source 1..41: mol_type = other DNA
  misc_feature 1..41: note = T17
  modified_base 40: mod_base = OTHER; note = deoxyinosine
ccagcattatgaaagtgccacgtgcaccattggtgcacgng

SEQ ID NO: 44 - DNA, 18 bp, synthetic construct
  source 1..18: mol_type = other DNA
  misc_feature 1..18: note = P11
  misc_feature 1: note = 5' phosphorothioate linkage
tcactttcataatgctgg

SEQ ID NO: 45 - DNA, 18 bp, synthetic construct
  source 1..18: mol_type = other DNA
  misc_feature 1..18: note = P12
  misc_feature 1: note = 5' phosphorothioate linkage
acactttcataatgctgg

SEQ ID NO: 46 - DNA, 18 bp, synthetic construct
  source 1..18: mol_type = other DNA
  misc_feature 1..18: note = P13
  misc_feature 1: note = 5' phosphorothioate linkage
ccactttcataatgctgg

SEQ ID NO: 47 - DNA, 18 bp, synthetic construct
  source 1..18: mol_type = other DNA
  misc_feature 1..18: note = P14
  misc_feature 1: note = 5' phosphorothioate linkage
gcactttcataatgctgg

SEQ ID NO: 48 - DNA, 18 bp, synthetic construct
  source 1..18: mol_type = other DNA
  misc_feature 1..18: note = P15
  misc_feature 1: note = 5' Rp phosphorothioate linkage
  modified_base 4..7: mod_base = OTHER; note = 5'-Rp phosphorothioate linkage
  modified_base 12..13: mod_base = OTHER; note = 5'-Rp phosphorothioate linkage
  modified_base 15..16: mod_base = OTHER; note = 5'-Rp phosphorothioate linkage
  modified_base 9..10: mod_base = OTHER; note = 5'-Rp phosphorothioate linkage
tcactttcataatgctgg

SEQ ID NO: 49 - DNA, 18 bp, synthetic construct
  source 1..18: mol_type = other DNA
  misc_feature 1..18: note = P16
  misc_feature 1: note = 5' Rp phosphorothioate linkage
  modified_base 2..3: mod_base = OTHER; note = 5'-Rp phosphorothioate linkage
  modified_base 8..9: mod_base = OTHER; note = 5'-Rp phosphorothioate linkage
  modified_base 10..12: mod_base = OTHER; note = 5'-Rp phosphorothioate linkage
acactttcataatgctgg

SEQ ID NO: 50 - DNA, 18 bp, synthetic construct
  source 1..18: mol_type = other DNA
  misc_feature 1..18: note = P17
  modified_base 1..2: mod_base = OTHER; note = 5'-Rp phosphorothioate linkage
  modified_base 3..4: mod_base = OTHER; note = 5'-Rp phosphorothioate linkage
  modified_base 7..8: mod_base = OTHER; note = 5'-Rp phosphorothioate linkage
  modified_base 14..15: mod_base = OTHER; note = 5'-Rp phosphorothioate linkage
  misc_feature 1: note = 5' Rp phosphorothioate linkage
ccactttcataatgctgg

SEQ ID NO: 51 - DNA, 18 bp, synthetic construct
  source 1..18: mol_type = other DNA
  misc_feature 1..18: note = P18
  misc_feature 1: note = 5' Rp phosphorothioate linkage
  modified_base 13..14: mod_base = OTHER; note = 5'-Rp phosphorothioate linkage
  modified_base 16..18: mod_base = OTHER; note = 5'-Rp phosphorothioate linkage
gcactttcataatgctgg

SEQ ID NO: 52 - DNA, 31 bp, synthetic construct
  source 1..31: mol_type = other DNA
  misc_feature 1..31: note = T18
  modified_base 30: mod_base = OTHER; note = 2'-deoxyinosine
aaaaaaaacacgtgcaccattggtgcacgng

SEQ ID NO: 53 - DNA, 31 bp, synthetic construct
  source 1..31: mol_type = other DNA
  misc_feature 1..31: note = T19
  modified_base 30: mod_base = OTHER; note = 2'-deoxyinosine
ttttttttcacgtgcaccattggtgcacgng

SEQ ID NO: 54 - DNA, 31 bp, synthetic construct
  source 1..31: mol_type = other DNA
  misc_feature 1..31: note = T20
  modified_base 30: mod_base = OTHER; note = 2'-deoxyinosine
ggggggggcacgtgcaccattggtgcacgng

SEQ ID NO: 55 - DNA, 31 bp, synthetic construct
  source 1..31: mol_type = other DNA
  misc_feature 1..31: note = T21
  modified_base 30: mod_base = OTHER; note = 2'-deoxyinosine
cccccccccacgtgcaccattggtgcacgng

SEQ ID NO: 56 - DNA, 31 bp, synthetic construct
  source 1..31: mol_type = other DNA
  misc_feature 1..31: note = T22
  modified_base 30: mod_base = OTHER; note = 2'-deoxyinosine
ctgactgacacgtgcaccattggtgcacgng

SEQ ID NO: 57 - DNA, 31 bp, synthetic construct
  source 1..31: mol_type = other DNA
  misc_feature 1..31: note = T23
  modified_base 30: mod_base = OTHER; note = 2'-deoxyinosine
catgcatgcacgtgcaccattggtgcacgng

SEQ ID NO: 58 - DNA, 31 bp, synthetic construct
  source 1..31: mol_type = other DNA
  misc_feature 1..31: note = T24
  modified_base 30: mod_base = OTHER; note = 2'-deoxyinosine
tacctaggcacgtgcaccattggtgcacgng

SEQ ID NO: 59 - DNA, 44 bp, synthetic construct
  source 1..44: mol_type = other DNA
  misc_feature 1..44: note = T25
  misc_feature 1: note = 5' biotin
  modified_base 43: mod_base = OTHER; note = 2'-deoxyinosine
cgcaagaagaagagcaaacgccacgtgcaccattggtgcacgng

SEQ ID NO: 60-73 - skipped (14 entries)

SEQ ID NO: 74 - DNA, 21 bp, synthetic construct
  source 1..21: mol_type = other DNA
  misc_feature 1..21: note = Vitravene
gcgtttgctcttcttcttgcg

SEQ ID NO: 75 - protein, 782 aa, synthetic construct
  REGION 1..782: note = KOD
MILDTDYITEDGKPVIRIFKKENGEFKIEYDRTFEPYFYALLKDDSAIEEVKKITAERHGTVVTVKRVEKVQKKFLGRPVEVWKLYFTHPQDVPAIRDKIREHPAVIDIYEYDIPFAKRYLIDKGLVPMEGDEELKMLAFAIATLYEEGEEFAEGPILMISYADEEGARVITWKNVDLPYVDVVSTEREMIKRFLRVVKEKDPDVLITYNGDNFDFAYLKKRCEKLGINFALGRDGSEPKIQRMGDRFAVEVKGRIHFDLYPVIRRTINLPTYTLEAVYEAVFGQPKEKVYAEEITTAWETGENLERVARYSMEDAKVTYELGKEFLPMEAQLSRLIGQSLWDVSRSSTGNLVEWFLLRKAYERNELAPNKPDEKELARRRQSYEGGYVKEPERGLWENIVYLDFRSLYPSIIITHNVSPDTLNREGCKEYDVAPQVGHRFCKDFPGFIPSLLGDLLEERQKIKKKMKATIDPIERKLLDYRQRAIKILANSYYGYYGYARARWYCKECAESVTAWGREYITMTIKEIEEKYGFKVIYSDTDGFFATIPGADAETVKKKAMEFLKYINAKLPGALELEYEGFYERGFFVTKKKYAVIDEEGKITTRGLEIVRRDWSEIAKETQARVLEALLKDGDVEKAVRIVKEVTEKLSKYEVPPEKLVIHEQITRDLKDYKATGPHVAVAKRLAARGVKIRPGTVISYIVLKGSGRIGDRAIPFDEFDPTKHKYDAEYYIENQVLPAVERILRAFGYRKEDLRYQKTRQVGLSAWLKPKGTLEHHHHHH

SEQ ID NO: 76 - protein, 783 aa, synthetic construct
  REGION 1..783: note = 9oN
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFAIATLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRLIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKKLEHHHHHH

SEQ ID NO: 77 - protein, 549 aa, synthetic construct
  REGION 1..549: note = Stoffel fragment
MALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQIELRVLAHLSGDENLIRVFQEGRDIHTETASWMFGVPREAVDPLMRRAAKTINFGVLYGMSAHRLSQELAIPYEEAQAFIERYFQSFPKVRAWIEKTLEEGRRRGYVETLFGRRRYVPDLEARVKSVREAAERMAFNMPVQGTAADLMKLAMVKLFPRLEEMGARMLLQVHDELVLEAPKERAEAVARLAKEVMEGVYPLAVPLEVEVGIGEDWLSAKELEHHHHHH

SEQ ID NO: 78 - protein, 549 aa, synthetic construct
  REGION 1..549: note = SFM4-6
MALEEAPWPPPEGAFVGFVLSRKEPMWADLLALAAARGGRVHRAPEPYKALRDLKEARGLLAKDLSVLALREGLGLPPGDDPMLLAYLLDPSNTTPEGVARRYGGEWTEEAGERAALSERLFANLWGRLEGEERLLWLYREVERPLSAVLAHMEATGVRLDVAYLRALSLEVAEEIARLEAEVFRLAGHPFNLNSRDQLERVLFDELGLPAIGKTEKTGKRSTSAAVLEALREAHPIVEKILQYRELTKLKSTYIDPLPDLIHPRTGRLHTRFNQTATATGRLSSSDPNLQNIPVRTPLGQRIRRAFIAEEGWLLVALDYSQEGLRVLAHLSGDENLIRVFQEGRDIHTETASWMFGVPREAVNPMMRRAAKTINFGVLYGMSAHRLSQKLAIPYEEAQAFIERYFQSFPKVRAWIEKTLEEGRRRGYVETLFGRRRYVPDLEARVKSVRNAAERRAFNMPVQGTAADLMKLAMVKLFPRLEEMGARMLLQVHDELVLEAPKERAEAVARLAKEVMEGVYPLAVPLEVEVGIGEDWLSAKELEHHHHHH

SEQ ID NO: 79 - protein, 548 aa, synthetic construct
  REGION 1..548: note = TfPol
MPREEAPWPPPEGAFVGFLLSRKEPMWAELLALAAAAEGRVHRATSPVEALADLKEARGFLAKDLAVLALREGVALDPTDDPLLVAYLLDPANTNPEGVARRYGGEFTEDAAERALLSERLFQNLFPRLSEKLLWLYQEVERPLSRVLAHMEARGVRLDVPLLEALSFELEKEMERLEGEVFRLAGHPFNLNSRDQLERVLFDELGLTPVGRTEKTGKRSTAQGALEALRGAHPIVELILQYRELSKLKSTYLDPLPRLVHPRTGRLHTRFNQTATATGRLSSSDPNLQNIPVRTPLGQRIRKAFVAEEGWLLLAADYSQIELRVLAHLSGDENLKRVFREGKDIHTETAAWMFGLDPALVDPKMRRAAKTVNFGVLYGMSAHRLSQELGIDYKEAEAFIERYFQSFPKVRAWIERTLEEGRTRGYVETLFGRRRYVPDLASRVRSVREAAERMAFNMPVQGTAADLMKIAMVKLFPRLKPLGAHLLLQVHDELVLEVPEDRAEEAKALVKEVMENTYPLDVPLEVEVGVGRDWLEAKGDLEHHHHHH

SEQ ID NO: 80 - protein, 560 aa, synthetic construct
  REGION 1..560: note = TfPol*
MGSSHHHHHHSSGLVPRGSHMPREEAPWPPPEGAFVGFLLSRKEPMWAELLALAAAAEGRVHRATSPVEALADLKEARGFLAKDLAVLALREGVALDPTDDPLLVAYLLDPANTNPEGVARRYGGEFTEDAAERALLSERLFQNLFPRLSEKLLWLYQEVERPLSRVLAHMEARGVRLDVPLLEALSFELEKEMERLEGEVFRLAGHPFNLNSRDQLERVLFDELGLTPVGRTEKTGKRSTAQGALEALRGAHPIVELILQYRELSKLKSTYLDPLPRLVHPRTGRLHTRFNQTATATGRLSSSDPNLQNIPVRTPLGQRIRKAFVAEEGWLLLAADYSQEGLRVLAHLSGDENLKRVFREGKDIHTETAAWMFGLDPALVNPMMRRAAKTVNFGVLYGMSAHRLSQVLGIDYKEAEAFIERYFQSFPKVRAWIERTLEEGRTRGYVETLFGRRRYVPDLASRVRSVRNAAERRAFNMPVQGTAADLMKIAMVKLFPRLKPLGAHLLLQVHDELVLEVPEDRAEEAKALVKEVMENTYPLDVPLEVEVGVGRDWLEAKGD

SEQ ID NO: 81 - protein, 560 aa, synthetic construct
  REGION 1..560: note = MhPol*
MGSSHHHHHHSSGLVPRGSHMAEEAPWPPPPEAFLGYVLDRPQPMWAELKGLAGAWEGRVARGPARAKELARFEAVHALQAKDLTVWARREGVRVQPGEDPLLLAYLYDPTNSDPAATVRRYGAGDWSEDPAARALAAAELWRYLGERLAGEEALWWLYREVERPLAGVLAEMEHAGVRVDVAYLEALSAELGREIAAIEAEVHRLAGRAFNLNSRDQLEVILYDELGLTPTRRTQKTGRRSTSAAALEALVGAHPIVERILAYRELSKLKGTYLDPLPRLVHPATGRIHTRYHQTGTATGRLSSSDPNLQNIPVRTEVGRRIRRAFVAEPGYVLVAADYSQEGLRVLAHLSGDENLKRVFQERRDIHTQTASWMFGVPPEAVNPMRRRAAKTVNFGVLYGMSPHRLSRVLGIEYAEAERFIQRYFESYPRVQAYIERTLEQAREKGYVETLFGRRRYIPDIRSRNRNVRNAAERRAFNMPVQGTAADLMKLAMVKLAPEIRSLGARLILQVHDELVLEAPQERAEAVARVVREVMEGAWALDVPLEVEVGIGENWLEAK

SEQ ID NO: 82 - protein, 548 aa, synthetic construct
  REGION 1..548: note = MhPol
MAEEAPWPPPPEAFLGYVLDRPQPMWAELKGLAGAWEGRVARGPARAKELARFEAVHALQAKDLTVWARREGVRVQPGEDPLLLAYLYDPTNSDPAATVRRYGAGDWSEDPAARALAAAELWRYLGERLAGEEALWWLYREVERPLAGVLAEMEHAGVRVDVAYLEALSAELGREIAAIEAEVHRLAGRAFNLNSRDQLEVILYDELGLTPTRRTQKTGRRSTSAAALEALVGAHPIVERILAYRELSKLKGTYLDPLPRLVHPATGRIHTRYHQTGTATGRLSSSDPNLQNIPVRTEVGRRIRRAFVAEPGYVLVAADYSQEGLRVLAHLSGDENLKRVFQERRDIHTQTASWMFGVPPEAVNPMRRRAAKTVNFGVLYGMSPHRLSRVLGIEYAEAERFIQRYFESYPRVQAYIERTLEQAREKGYVETLFGRRRYIPDIRSRNRNVRDAAERMAFNMPVQGTAADLMKLAMVKLAPEIRSLGARLILQVHDELVLEAPQERAEAVARVVREVMEGAWALDVPLEVEVGIGENWLEAKLEHHHHHH

SEQ ID NO: 83 - protein, 782 aa, synthetic construct
  REGION 1..782: note = KOD DGLNK
MILDTDYITEDGKPVIRIFKKENGEFKIEYDRTFEPYFYALLKDDSAIEEVKKITAERHGTVVTVKRVEKVQKKFLGRPVEVWKLYFTHPQDVPAIRDKIREHPAVIDIYEYDIPFAKRYLIDKGLVPMEGDEELKMLAFDIETLYEEGEEFAEGPILMISYADEEGARVITWKNVDLPYVDVVSTEREMIKRFLRVVKEKDPDVLITYDGDNFDFAYLKKRCEKLGINFALGRDGSEPKIQRMGDRFAVEVKGRIHFDLYPVIRRTINLPTYTLEAVYEAVFGQPKEKVYAEEITTAWETGENLERVARYSMEDAKVTYELGKEFLPMEAQLSRLIGQSLWDVSRSSTGNLVEWFLLRKAYERNELAPNKPDEKELARRRQSYEGGYVKEPERGLWENIVYLDFRSLGPSIIITHNVSPDTLNREGCKEYDVAPQVGHRFCKDFPGFIPSLLGDLLEERQKIKKKMKATIDPIERKLLDYRQRLIKILANSYYGYYGYARARWYCKECAESVTAWGREYITMTIKEIEEKYGFKVIYSDTDGFFATIPGADAETVKKKAMEFLKYINAKLPGALELEYEGFYERGFFVTKKKYAVIDEEGKITTRGLEIVRRNWSEIAKETQARVLEALLKDGDVEKAVRIVKEVTEKLSKYEVPPEKLVIHKQITRDLKDYKATGPHVAVAKRLAARGVKIRPGTVISYIVLKGSGRIGDRAIPFDEFDPTKHKYDAEYYIENQVLPAVERILRAFGYRKEDLRYQKTRQVGLSAWLKPKGTLEHHHHHH

SEQ ID NO: 84 - protein, 233 aa, synthetic construct
  REGION 1..233: note = TmEndo
MDYRQLHRWDLPPEEAIKVQNELRKKIKLTPYEGEPEYVAGVDLSFPGKEEGLAVIVVLEYPSFKILEVVSERGEITFPYIPGLLAFREGPLFLKAWEKLRTKPDVVVFNGQGLAHPRKLGIASHMGLFIEIPTIGVAKSRLYGTFKMPEDKRCSWSYLYDGEEIIGCVIRTKEGSAPIFVSPGHLMDVESSKRLIKAFTLPGRRIPEPTRLAHIYTQRLKKGLFLEHHHHHH

SEQ ID NO: 85 - protein, 234 aa, synthetic construct
  REGION 1..234: note = TnEndoV
MHMTYKKLHEWDLSPEEAMKIQNVLREKILFKPFEGEPKYVAGVDLSFPKREEGLAVIVVMEYPTFKIVELVSERGKVDFPYIPGLLAFREGPLFLKAWEKLKTKPDVVVFDGQGIAHPRKLGIASHMGLFIEIPTIGVAKSRLYGTYREPENRRCSWSYLYDNEEIIGCVMRTREGSAPIFVSPGHLIDVESSIRLVKSFTLPGRRLPEPTRMAHIYTQRLKKGLLEHHHHHH

SEQ ID NO: 86 - protein, 235 aa, synthetic construct
  REGION 1..235: note = PtEndoV
MHMKYEKLHEWNVSVEEAIKIQNQLRSKIKLMPLKEEKIRYVAGVDLSFPEPNLGYAVIVVIDLFNMSIVENVAAKEKVVFDYIPGLLAFREGPVFLKAWENLKTKPNLVVFDGHGIAHPRGLGIASHMGLWINLPTIGVAKSKLYGFYKGLQQAKWSEVPLLDPSGNQIGIVIRTKENADPIFVSPGHLCDLDSASKIVKKMCLENNRLPEPTRLAHLLTQKIHRRLEHHHHHH

SEQ ID NO: 87 - protein, 236 aa, synthetic construct
  REGION 1..236: note = TmeEndoV
MHMEYKKLHSWKLIPEKAIELQKLLSKKLTFKPLSKEINLVAGVDLSFVKDYGLAVIVILDKNMELIKVYHHIEKITFPYIPGLLAFREGPIFLKAWKKVNHNVDVVFFDGHGISHPRSMGIASHMGLWIEQPTIGIAKKILFGNYVEPENKKFSFTYITYKNQKIGIVLRSRENVKPIFISPGNLITLDESLELTKQFITKYKLPEPTRLAHKYSQLLKKQFNEELQLEHHHHHH

SEQ ID NO: 88 - protein, 233 aa, synthetic construct
  REGION 1..233: note = TaEndoV
MHMLYKKLHDWNISPKKAIDIQSTLKKLLKFEKIEKEISLVAGVDLSFFKEYGLAVIVVLDYDFNIREIVHHVEKVSYPYIPGLLAFREGPIFIKAWEKLKTDVDIVFFDGHGIAHPRKLGIASHMGLWIEKATIGIAKSKLIGNYKEPGKKRGNFSYLEYDGEKIGIVYRSRNNVKPIFISPGYLIDLESSLKITKSFITKYRLPEPTRLAHLYTQKIKKEHLSLEHHHHHH

SEQ ID NO: 89 - RNA, 18 nt, synthetic construct
  source 1..18: mol_type = other RNA
  misc_feature 1..18: note = Spinraza sequence
  misc_feature 1..18: note = PS linkage between nucleotides
  modified_base 1..2: mod_base = OTHER; note = 2' MOE
  modified_base 4..8: mod_base = OTHER; note = 2' MOE
  modified_base 13: mod_base = OTHER; note = 2' MOE
  modified_base 15..16: mod_base = OTHER; note = 2' MOE
  modified_base 10: mod_base = OTHER; note = 2' MOE
tcactttcataatgctgg

SEQ ID NO: 90 - DNA, 23 bp, synthetic construct
  source 1..23: mol_type = other DNA
  misc_feature 1..23: note = Example template
  misc_feature 1: note = 5' Biotin
  modified_base 22: mod_base = OTHER; note = deoxyinosine
tgaaagtgagatctaggctganc

SEQ ID NO: 91 - DNA, 32 bp, synthetic construct
  misc_feature 1..23: note = DNA
  misc_feature 24..32: note = RNA
  source 1..32: mol_type = other DNA
  misc_feature 23..32: note = RNA
  modified_base 22: mod_base = OTHER; note = deoxyinosine
  misc_feature 1..32: note = Example extended product
  modified_base 24..25: mod_base = OTHER; note = 2' MOE
  modified_base 27..32: mod_base = OTHER; note = 2' MOE
  modified_base 23..32: mod_base = OTHER; note = Phosphorothioate linkage
  misc_feature 1: note = 5' biotin
agaaagtgagatctaggctganctcactttct

SEQ ID NO: 92 - DNA, 11 bp, synthetic construct
  source 1..11: mol_type = other DNA
  misc_feature 1..11: note = Primer (lane 6)
  modified_base 10: mod_base = OTHER; note = deoxyinosine
tggtgcacgng

SEQ ID NO: 93 - DNA, 29 bp, synthetic construct
  source 1..29: mol_type = other DNA
  misc_feature 1..29: note = Template (lane 6)
ccagcattatgaaagtgacacgtgcacca

SEQ ID NO: 94 - DNA, 37 bp, synthetic construct
  source 1..37: mol_type = other DNA
  misc_feature 1..37: note = Template (Lane 10)
  modified_base 36: mod_base = OTHER; note = deoxyinosine
ccagcattatgaaagtgacacgtgccattggcacgng

SEQ ID NO: 95 - DNA, 17 bp, synthetic construct
  source 1..17: mol_type = other DNA
  misc_feature 1..17: note = b (product)
tcactttcataatgctg

SEQ ID NO: 96 - DNA, 16 bp, synthetic construct
  source 1..16: mol_type = other DNA
  misc_feature 1..16: note = b (product)
tcactttcataatgct

SEQ ID NO: 97 - DNA, 18 bp, synthetic construct
  source 1..18: mol_type = other DNA
  misc_feature 1..18: note = c (product)
  misc_feature 1: note = 5' OH
tcactttcataatgctgg

SEQ ID NO: 98 - DNA, 15 bp, synthetic construct
  source 1..15: mol_type = other DNA
  misc_feature 1..14: note = c (product)
tcactttcataatgc

SEQ ID NO: 99 - DNA, 14 bp, synthetic construct
  source 1..14: mol_type = other DNA
  misc_feature 1..14: note = d (product)
tcactttcataatg

SEQ ID NO: 100 - DNA, 13 bp, synthetic construct
  source 1..13: mol_type = other DNA
  misc_feature 1..13: note = e (product)
tcactttcataat

SEQ ID NO: 101 - DNA, 12 bp, synthetic construct
  source 1..12: mol_type = other DNA
  misc_feature 1..12: note = f (product)
tcactttcataa

SEQ ID NO: 102 - DNA, 11 bp, synthetic construct
  source 1..11: mol_type = other DNA
  misc_feature 1..11: note = g (product)
tcactttcata

SEQ ID NO: 103 - DNA, 21 bp, synthetic construct
  source 1..21: mol_type = other DNA
  misc_feature 1..21: note = Vitravene product a
  modified_base 1..21: mod_base = OTHER; note = phosphorothioate linkage
  misc_feature 1: note = 5' OH
  misc_feature 1: note = 5' phosphorothioate linkage
gcgtttgctcttcttcttgcg

SEQ ID NO: 104 - DNA, 21 bp, synthetic construct
  source 1..21: mol_type = other DNA
  misc_feature 1..21: note = Vitravene product b
  modified_base 1..21: mod_base = OTHER; note = phosphorothioate linkage
gcgtttgctcttcttcttgcg

SEQ ID NO: 105 - DNA, 22 bp, synthetic construct
  source 1..22: mol_type = other DNA
  misc_feature 1..22: note = Vitravene product c
  modified_base 1..22: mod_base = OTHER; note = phosphorothioate linkage
  misc_feature 1: note = 5' OH
gcgtttgctcttcttcttgcgc